(19) United States
(12) Patent Application Publication
(10) Pub. No.: US 2025/0288667 A1
(43) Pub. Date: Sep. 18, 2025

(54) GENETICALLY ENGINEERED MUCOSAL-ASSOCIATED INVARIANT T (MAIT) CELLS FOR ADOPTIVE TRANSFER CELLTHERAPY

(71) Applicants: PLURI BIOTECH LTD., Haifa (IL); HADASIT MEDICAL RESEARCH SERVICES AND DEVELOPMENT LTD., JERUSALEM (IL)

(72) Inventors: Arthur MACHLENKIN, Gedera (IL); Michal LOTEM, SDEROT HAOMARIN (IL); Helena Lea SHAKED, RAANANA (IL); Shira KLEIN SILBERMAN, MODIIN (IL)

(21) Appl. No.: 18/866,750

(22) PCT Filed: Jul. 9, 2024
(86) PCT No.: PCT/IL2024/050670

§ 371 Date: Nov. 18, 2024

Related U.S. Application Data
(60) Provisional application No. 63/525,836, filed on Jul. 10, 2023.

(51) Int. Cl. A61K 40/11; A61K 40/32; A61K 40/42; A61P 35/00

(52) U.S. Cl. CPC A61K 40/11; A61K 40/32; A61K 40/4267; A61K 40/4269; A61P 35/00

(57) ABSTRACT

The present disclosure relates in general to the field of genetic engineering of immune cells, specifically to mucosal-associated invariant T (MAIT) cells genetically engineered to express an exogeneous T cell receptor (TCR) and uses thereof. More specifically, the invention in embodiments thereof relates to cell compositions adapted for adoptive transfer cell therapy (ACT) providing for improved therapeutic modalities.

### FIELD OF THE INVENTION

The present disclosure relates in general to the field of genetic engineering of immune cells. More specifically, the present disclosure provides mucosal-associated invariant T (MAIT) cells genetically engineered to express an exogeneous T cell receptor (TCR) and uses thereof.

### BACKGROUND OF THE INVENTION

Mucosal-associated invariant T (MAIT) cells were first identified in humans, mice, and cattle as a population of αβ T cells enriched in the double-negative (CD4⁻CD8⁻) subset expressing an invariant Vα7.2-Jα33 T cell receptor (TCR) in humans. The term MAIT was established due to the relative enrichment of these T cells within mucosal tissues. The restricting element of MAIT cells is the MHC-1b molecule MR1 (MHC-related 1), which presented intermediates in the vitamin B ((both riboflavin (vitamin B2) and folic acid (vitamin B9)) synthesis pathway to MAIT cells. Potent stimulatory ligands in the riboflavin synthesis pathway include 5-(2-oxopropylideneamino)-6-D-ribitylaminouracil (5-OP-RU) and 5-(2-oxoethylideneamino)-6-D-ribitylaminouracil (5-OE-RU) that are produced by a wide variety of bacteria, mycobacteria and yeasts during riboflavin synthesis. This pathway is absent from mammals; therefore, its immune detection allows effective host-pathogen discrimination.

It has been found that MAIT cells can recognize cells infected by bacteria and produce IFN-γ in response. It has been demonstrated that MAIT cells can protect mice from bacterial infection. Early phenotypic work established that human MAIT cells are CD8⁺ or double negative, have a primarily CCR7⁻ effector memory phenotype, and express high levels of CD161. Detailed phenotyping of MAIT cells also demonstrated that they share several characteristics with invariant natural killer T (iNKT) cells, including the expression of PLZF, a transcription factor that governs the innate-like functionality of iNKT cells. Expression of PLZF similarly imparts MAIT cells with innate-like functionality, as evidenced by the ability of cytokines to induce IFN-γ production in the absence of TCR stimulation.

Subsequent studies found that human MAIT cells do not express a single invariant TCR, but restricted TCRs comprising Vα7.2-Jα33, Vα7.2-Jα12, or Vα7.2-Jα20, which are predominantly associated with a limited repertoire of the human β chains such as Vβ2/Vβ13. Thus, current understanding views MAIT cells as T cells that (a) express a semi-invariant Vα7.2-Jα33/12/20 TCR, (b) are activated by microbial vitamin B metabolite antigens presented by MR1 to execute both type 1 and type 17 effector functions, and (c) exhibit innate-like characteristics, governed by expression of PLZF, including the ability to be activated by cytokines independent of their TCR.

MAIT cells contrast with conventional T cells which possess highly variable TCRs capable of targeting a vast array of peptide epitopes produced by viruses, bacteria and malignant cells. Conventional T cells therefore have exquisite specificity for individual peptides, and individual clones may undergo massive expansion to provide T cell memory. However, at the first encounter with a pathogen the frequency of any individual peptide-specific T cell will be very low. In contrast, the MAIT cell TCR provides an innate-like capacity to respond to a specific set of ligands without the need for expansion.

Several properties of MAIT cells imply fundamental roles in mammalian immunity. First, MAIT cells have an intrinsic effector-memory phenotype, usually CD45RA⁻CD45RO⁺CD95^{Hi}CD62L^{Lo}CD44^{Hi}, with capacity for rapid secretion of several pro-inflammatory cytokines. Second, MAIT cells are remarkably abundant in human tissues, typically comprising 1-4% of all T cells in peripheral blood and up to 10% of airway T cells and 20-40% of liver T cells. Moreover, as each TCR recognizes the same ligand, early in an immune response, MAIT cells will markedly exceed the numbers of conventional antigen-specific T cells responding to cognate antigens.

Recent studies using single-cell RNA sequencing technology and immunology techniques have revealed that MAIT cells demonstrate marked heterogeneity that recapitulates conventional T cell biology. It was demonstrated that this marked heterogeneity includes distinct CD4⁺ and CD8⁺ lineages, as well as “killer,” “helper,” and “regulatory” cell phenotypes—an indication that MAIT cells exercise complex functions.

The conservation and abundance of MAIT cells is likely explained by their broad range of functionality attributable to different modes of activation, each triggering a distinct transcriptomic program. Because of their capability for diverse functional responses in diverse immunological contexts, these intriguing cells now appear to be multifunctional effectors central to the interface of innate and adaptive immunity. Already three major functions—antibacterial host defense, antiviral host defense, tissue repair and homeostasis—have been described for these intriguing cells, but it is likely other functions remain to be discovered.

WO 2021/113759 discloses populations of T cells expressing a chimeric antigen receptor (CAR), wherein said T cells are placental T cells derived from cord blood, placental perfusate, or a mixture thereof. The use of MAIT cells is not suggested, nor is the isolation of specific populations derived from placental intervillous blood.

Parrot et al, (JCI Insight. 2021; 6 (5): e140074) and Healy et al (JHEP Reports 2021; vol. 3) report on experiments performed with human MAIT cells optionally engineered to recognize a viral antigen. In particular, MAIT cells were obtained from peripheral blood, expanded in culture and engineered to express a hepatitis B virus (HBV)-specific TCR. The publications disclose that the ex vivo expanded MAIT cells have been shown to be CD161 low after expansion, and downregulate CD161 in response to antigenic stimulation. The authors further report of the development of a progressively more differentiated phenotype over time during all of their tested expansion protocols, which may compromise the function and survival of adoptively transferred MAIT cells in vivo.

WO 2024/023826, to some of the present inventors and co-workers, discloses engineered TCRs directed to cancer testis antigen New York Esophageal Squamous Cell Carcinoma-1 (NY-ESO-1), useful in the treatment of cancer.

U.S. Pat. No. 11,939,562 discloses a three-dimensional (3D) bioreactor for large scale expansion of immune cells and methods of use.

Thus, it is of interest to further study and develop MAIT cell compositions for various therapeutic uses.

### SUMMARY OF THE INVENTION

The present disclosure relates in general to the field of genetic engineering of immune cells, and specifically to mucosal-associated invariant T (MAIT) cells genetically engineered to express an exogeneous T cell receptor (TCR) and uses thereof. More specifically, the invention in embodiments thereof relates to cell compositions adapted for adoptive transfer cell therapy (ACT) providing for improved therapeutic modalities.

In various embodiments, compositions and methods in accordance with the invention may be used in the treatment of cancer and other conditions associated with expression of an HLA class I-restricted antigen. As further disclosed herein, compositions and methods in accordance with the invention are advantageously amenable for use even in connection with patient populations not otherwise considered amenable for treatment with immunotherapy-based methods (e.g. due to primary or acquired resistance).

In one aspect, there is provided a cell composition comprising a population of engineered MAIT cells expressing an exogenous TCR, wherein the MAIT cells are derived from placenta, optionally further comprising a pharmaceutically acceptable carrier.

In one embodiment, the MAIT cells are advantageously derived from placental intervillous blood (IVB). In another embodiment, the composition is adapted for adoptive transfer cell therapy (ACT). In another embodiment, the composition comprises 10⁹-10¹¹ viable cells of said engineered MAIT cell population. In another embodiment said composition comprises at least 90% TCRVα7.2⁺ CD161^{high} cells. In another embodiment, the composition is as disclosed and further characterized herein.

In another embodiment, said TCR recognizes a tumor antigen. In some embodiments, the tumor antigen may be selected from the group consisting of: NY-ESO-1, KRAS, p53, PIK3CA, PTEN, ERBB2 (HER2), AFP, KK-LC-1, RAC1-P29S, LAGE-1A, COL6A3, HA-2, HERV-E, BRAF, gp100, alpha-fetoprotein, Desmoyokin/AHNAKS2580F, Cancer/testis antigen 1 ERBB2H473Y, ERBB2IPE805G, minor H antigen (HA-1), PRAME, TPBG, 5T4, MAGEA1, MAGE-A3/A6, MAGEA4/8, Melan-A/MART-1, NRAS and Wilms tumor 1 (WT-1). Each possibility represents a separate embodiment of the invention. According to an exemplary embodiment, said tumor antigen is selected from the group consisting of NY-ESO-1 and LAGE-1A. In a particular embodiment said tumor antigen is NY-ESO-1. In another embodiment said TCR is capable of specific binding to an HLA-A2-presented epitope. According to an exemplary embodiment, said TCR comprises a TCR α chain comprising a CDR1 having the amino acid sequence of SEQ ID NO: 1, a CDR2 having the amino acid sequence of SEQ ID NO: 2, and a CDR3 having the amino acid sequence of SEQ ID NO: 3, and a TCR β chain comprising a CDR1 having the amino acid sequence of SEQ ID NO: 4, a CDR2 having the amino acid sequence of SEQ ID NO: 5, and a CDR3 having the amino acid sequence of SEQ ID NO: 6 as set forth in Table 1 below. In another particular embodiment, said TCR comprises a TCR α chain having the amino acid sequence as set forth in SEQ ID NO: 7, optionally excluding the signal peptide at positions 1-20 thereof, and a TCR β chain having the amino acid sequence as set forth in SEQ ID NO: 8, optionally excluding the signal peptide at positions 1-21 thereof. In another embodiment, the antigen or TCR is as disclosed and further characterized herein.

In another embodiment, cell compositions of the invention are for use in therapy. In another embodiment, the use is in treating a subject having a tumor or malignancy. In another embodiment, the subject is afflicted with a tumor selected from the group consisting of: melanoma, breast cancer, colon cancer, kidney cancer, liver cancer, lung cancer, ovary cancer, pancreatic cancer, prostate cancer, uterine cancer, cervical cancer, bladder cancer, stomach cancer, head and neck cancer, brain cancer, skin cancer and sarcoma. Each possibility represents a separate embodiment of the invention. In another embodiment wherein the subject is afflicted with a treatment-resistant tumor or is not otherwise amenable for treatment with an immunotherapy comprising chimeric antigen receptor (CAR) T cells and/or therapeutic antibodies. In another embodiment the use is in treating a subject afflicted with a condition associated with expression of an HLA class I-restricted antigen. In another embodiment said antigen is a low-density antigen characterized by surface expression of less than 50 antigen molecules per cell (e.g. an epitope as disclosed herein presented in the context of an MHC molecule). In another embodiment, the use is as disclosed and further characterized herein. In another aspect, there is provided a cell composition adapted for ACT, the composition comprising a substantially purified population of MAIT cells engineered to express an exogenous TCR, wherein the TCR recognizes a cancer testis antigen. In another aspect, there is provided a cell composition adapted for ACT, the composition comprising a substantially purified population of MAIT cells engineered to express an exogenous TCR, wherein the TCR recognizes a tumor antigen selected from the group consisting of NY-ESO-1 and LAGE-1A. In another embodiment said TCR comprises a TCR α chain comprising a CDR1 having the amino acid sequence of SEQ ID NO: 1, a CDR2 having the amino acid sequence of SEQ ID NO: 2, and a CDR3 having the amino acid sequence of SEQ ID NO: 3, and a TCR β chain comprising a CDR1 having the amino acid sequence of SEQ ID NO: 4, a CDR2 having the amino acid sequence of SEQ ID NO: 5, and a CDR3 having the amino acid sequence of SEQ ID NO: 6. In another embodiment said TCR comprises a TCR α chain having the amino acid sequence as set forth in SEQ ID NO: 7, optionally excluding the signal peptide at positions 1-20 thereof, and a TCR β chain having the amino acid sequence as set forth in SEQ ID NO: 8, optionally excluding the signal peptide at positions 1-21 thereof. In another embodiment, the cell composition is as disclosed and further characterized herein.

In another aspect, there is provided a method of treating a subject afflicted with a condition associated with expression of an HLA class I-restricted antigen, comprising administering to the subject a cell composition as defined herein. In one embodiment, the cell composition comprises a population of engineered MAIT cells expressing an exogenous TCR, wherein the MAIT cells are derived from placenta, optionally further comprising a pharmaceutically acceptable carrier. In another embodiment, the cell composition is adapted for ACT and comprises a substantially purified population of MAIT cells engineered to express an exogenous TCR, wherein the TCR recognizes a tumor antigen selected from the group consisting of NY-ESO-1 and LAGE-1A.

In another aspect, the invention provides a method of treating a subject having a tumor or malignancy, comprising administering to said subject a cell composition as defined herein. In another aspect, the invention provides a method of treating a subject having a tumor or malignancy, comprising administering to said subject a cell composition comprising a population of engineered MAIT cells expressing an exogenous TCR, wherein the MAIT cells are derived from placenta, optionally further comprising a pharmaceutically acceptable carrier. In another aspect, the invention provides a method of treating a subject having a tumor or malignancy, comprising administering to said subject a cell composition adapted for ACT, the composition comprising a substantially purified population of MAIT cells engineered to express an exogenous TCR, wherein the TCR recognizes a tumor antigen selected from the group consisting of NY-ESO-1 and LAGE-1A.

In another aspect, there is provided a method of treating a subject afflicted with a condition associated with expression of an HLA class I-restricted antigen, comprising administering to the subject a cell composition comprising a population of engineered MAIT cells expressing an exogenous TCR, and a pharmaceutically acceptable carrier.

In one embodiment of the methods of the invention, the MAIT cells are derived from placenta. In another embodiment of the methods of the invention, the MAIT cells have been obtained from placental IVB. In another embodiment, the subject is afflicted with a tumor or malignancy, and said TCR recognizes a tumor antigen expressed by cells of the tumor or malignancy. In another embodiment the antigen is a cancer testis antigen. In another embodiment the antigen is selected from the group consisting of NY-ESO-1 and LAGE-1A. In another embodiment said antigen is NY-ESO-1. In another embodiment said subject is HLA-A2-positive and is afflicted with a tumor or malignancy expressing NY-ESO-1 and/or LAGE-1A. In another embodiment said subject is HLA-A2-positive and is afflicted with a tumor or malignancy expressing NY-ESO-1 and/or LAGE-1A, and said TCR comprises a TCR α chain comprising a CDR1 having the amino acid sequence of SEQ ID NO: 1, a CDR2 having the amino acid sequence of SEQ ID NO: 2, and a CDR3 having the amino acid sequence of SEQ ID NO: 3, and a TCR β chain comprising a CDR1 having the amino acid sequence of SEQ ID NO: 4, a CDR2 having the amino acid sequence of SEQ ID NO: 5, and a CDR3 having the amino acid sequence of SEQ ID NO: 6. In another embodiment said TCR comprises a TCR α chain having the amino acid sequence as set forth in SEQ ID NO: 7, optionally excluding the signal peptide at positions 1-20 thereof, and a TCR β chain having the amino acid sequence as set forth in SEQ ID NO: 8, optionally excluding the signal peptide at positions 1-21 thereof. In another embodiment the tumor is selected from the group consisting of: melanoma, myeloma, sarcoma, and bladder, brain, ovarian, lung, breast, synovial and prostate tumors. Each possibility represents a separate embodiment of the invention. In a particular embodiment said tumor is melanoma.

In another embodiment of the methods of the invention, said antigen is a low-density antigen characterized by surface expression of less than 50 antigen molecules per cell. In another embodiment the cell composition is adapted for ACT and the population comprises at least 10⁹ viable cells, of which at least 90% are TCR-Vα7.2⁺ CD161^{high}. In another embodiment the engineered MAIT cells are allogeneic to said subject. In another embodiment the engineered MAIT cells are partly histocompatible with said subject. In another embodiment the engineered MAIT cells are not histocompatible with said subject. In another embodiment the subject is afflicted with a treatment-resistant tumor or is not otherwise amenable for treatment with an immunotherapy comprising CAR T cells and/or therapeutic antibodies. Each possibility represents a separate embodiment of the invention. In another embodiment, the method is as disclosed and further characterized herein.

In another aspect, the invention provides a process for producing a cell composition adapted for ACT, the process comprising the steps of:
a. obtaining a MAIT-cell containing cell population,
b. incubating the cells in the presence of a MAIT cell antigen and IL-15, so as to produce a population of activated MAIT cells,
c. engineering the resulting activated MAIT cells to express an exogeneous TCR (eTCR),
d. expanding the resulting engineered MAIT cells to obtain a therapeutically effective amount of said cells, and
e. isolating the resulting engineered MAIT cells.

In another embodiment the MAIT-cell containing cell population is obtained from placenta. In another embodiment the MAIT-cell containing cell population is obtained from IVB. In another embodiment step d. (the expanding step) is performed so as to obtain expansion by a factor of at least 100-fold. In another embodiment step e. (the isolating step) comprises subjecting the resulting cells to positive selection of TCRVα7.2-expressing cells, so as to obtain a population of substantially purified engineered MAIT cells. In another embodiment the process is as disclosed and further characterized herein.

In another embodiment, the process provides a cell composition adapted for ACT, comprising a substantially purified population of at least 10⁹ viable cells of said engineered MAIT cells, of which at least 90% are TCR-Vα7.2⁺ CD161^{high}, and at least 70% are eTCR⁺. In another embodiment the process provides a cell composition as disclosed and further characterized herein. In another embodiment there is provided a cell composition produced by the process.

Other objects, features and advantages of the present invention will become clear from the following description and drawings.

### BRIEF DESCRIPTION OF THE DRAWINGS

Some embodiments of the engineered mucosal-associated invariant T (MAIT) cells and methods of use thereof, are herein described, by way of example only, with reference to the accompanying drawings. With specific reference now to the drawings in detail, it is stressed that the particulars shown are by way of example and for purposes of illustrative discussion of embodiments of the engineered MAIT cells and MAIT cells lacking an exogenous antigen receptor, and therapeutic uses thereof. In this regard, the description taken with the drawings makes apparent to those skilled in the art how embodiments of the engineered MAIT cells, and MAIT cells lacking an exogenous antigen receptor, and therapeutic uses thereof may be practiced.

FIGS. 1A-1B show MAIT % among placenta intervillous blood (IVB) mononuclear cells and peripheral blood mononuclear cells (PBMC), gated on CD3⁺ cells by flow cytometry. FIG. 1A shows a representative sample of MAIT cells from placenta IVB (“MAIT”), gated on CD3⁺ population, and identified by CD161 and TCR Vα7.2 staining. FIG. 1B shows the proportion of CD3⁺ placenta (IVB) and peripheral blood (PB) MAIT cells analyzed from 13 IVB donors and 11 PB donors. p=0.0159.

FIGS. 2A-C show the phenotype of MAIT cells based on CD62L and CD45RA expression. FIG. 2A shows a representative sample of peripheral blood-derived total T cells (left), peripheral MAIT cells (center) and placenta (IVB) derived MAIT cells (right). Effector memory cells (EM) are highlighted. CM, central memory. TemRA, terminally differentiated effector cells that re-express CD45RA. FIG. 2B shows the results of IVB- and peripheral blood-derived MAIT cells analyzed from 6 donors each. Shown are the % of the indicated population (EM—left, TemRA—right) out of the evaluated MAIT population. FIG. 2C similarly shows the results (% out of MAIT) for IVB-derived MAIT cells (IVB MAIT) and peripheral blood-derived T cells (PB T).

FIGS. 3A-3B show expression of chemokine receptors on peripheral blood MAIT cells or IVB MAIT cells by flow cytometry. FIG. 3A shows MAIT cells from placenta IVB and peripheral blood (PB), 6 donors each, analyzed for expression of CCR5 (top left), CCR6 (top right), CXCR4 (bottom left) and CXCR6 (bottom right). Cells were gated on MAIT cells and median fluorescence intensity (MFI) was analyzed for each marker. FIG. 3B presents the percentage of each receptor as evaluated in FIG. 3A out of the tested population of MAIT cells (% out of MAIT).

FIGS. 4A-4B—show expression of granzyme B and perforin in MAIT cells from placenta IVB and peripheral blood, as well as in total peripheral blood CD3⁺ cells. FIG. 4A shows a representative sample for the three cell types (placenta IVB—left, PB MAIT—center, PB CD3⁺—right). FIG. 5B shows MAIT cells from placenta IVB (IVB MAIT) and peripheral blood (PB MAIT) and total peripheral blood CD3⁺ cells (PB T), 6 donors each, stimulated with PMA/ionomycin for 4 hours and analyzed for intracellular expression of granzyme B and perforin. Cells were gated on MAIT cells or CD3⁺ cells, respectively. p=0.0001.

FIGS. 5A-5B—show cytokine production in placenta IVB MAIT cells and PB T cells (PB T). FIG. 5A shows a representative sample of placenta IVB cells (gated on MAIT population) and peripheral blood T cells, stained for IFNγ and TNFα. FIG. 5B shows quantitative analysis of cytokine production by placenta MAIT cells vs peripheral T cells (6 donors of each, % expression of the tested cytokine out of the analyzed cells are shown).

FIGS. 6A-6B show MDR1 expression in IVB MAIT cells and peripheral blood T cells. FIG. 6A shows a representative sample of placenta IVB cells (gated on MAIT population, dotted line) and peripheral blood T cells (PB CD3⁺, solid line). FIG. 6B presents a quantitative analysis of MDR1 protein expression on placenta MAIT cells (IVB MAIT) and peripheral T cells (PB T, 6 donors of each), as detailed in FIG. 6A.

FIGS. 7A-7C show MAIT cells activation and expansion. FIG. 7A shows the outline of the expansion process, in which IVB mononuclear cells were seeded in tissue culture plates and MAIT cells activation was induced by 5-OP-RU (250 ng/ml) and IL-15 (50 ng/ml). On day 3 and 5 post-activation, cells were counted and analyzed by flow cytometry for MAIT %, and fresh medium and IL-15 was added. On day 7, cells were separated with magnetic beads and anti-TCR Vα7.2 antibody, to further enrich for MAIT population; cells were cultured for another 3 days in the presence of IL-15. The results show MAIT % increase (FIG. 7B) and MAIT fold expansion (increase in MAIT absolute numbers relative to the starting numbers, FIG. 7C) throughout the culture.

FIGS. 8A-8B show RNAseq RNA expression analysis of expanded placenta MAIT cells vs peripheral T cells. FIG. 8A—Heatmap of top differentially expressed genes between MAIT and CD8⁺ T cells. Three left columns represent CD8⁺ T cells from 3 different donors; three right columns represent MAIT cells from 3 different placentas. The upper part shows top 23 genes with higher expression in peripheral T cells than in MAIT cells, the bottom part shows top 17 genes with higher expression in MAIT cells than in peripheral T cells. FIG. 8B—Volcano plot showing genes that are significantly upregulated (right) or downregulated (left) in MAIT cells vs regular CD8⁺ T cells.

FIGS. 9A-9B show LegendScreen™ protein expression analysis of expanded placenta MAIT cells and peripheral T cells. FIG. 9A plots median fluorescence intensity (MFI) of the markers in both cell populations; for each population, 2 donors were analyzed, and the average MFI is plotted. Markers that had consistently differential expression are highlighted by black circles or black diamonds (for chemokine receptors) FIG. 9B shows exemplary flow cytometry histograms for selected differentially expressed markers, wherein IVB MAIT cells are marked by a dotted line and PB CD8⁺ T cells are marked with a full line.

FIG. 10 shows expression of chemokine receptors that is higher on IVB MAIT cells (MAIT) as compared to conventional T cells (T cells).

FIG. 11 demonstrates that MAIT cells have lower allogeneic profile than peripheral blood T cells. Left panel-CD8⁺ T cell allo-activation (measured as % of CD25⁺ cells) in the presence of inactivated MAIT cells (MAIT) or T cells (T). Non-stimulated CD8⁺ T cells (“unstimulated”) and CD8⁺ T cell stimulated by anti-CD3/CD28 TransAct reagent (“stimulated”) as well as CD8⁺ T cells incubated with same-source lymphocytes (“self”) were used as controls. Right panel-CD8⁺ T cell proliferation of the same experiment, represented as % of cells after more than three divisions.

FIGS. 12A-12D—show phenotypic flow cytometry analysis comparisons between cord blood (CB) MAIT cells and IVB-MAIT cells (IVB). FIG. 12A shows expression of TCR Vα7.2 and CD161 of T cells in CB (right panel) and IVB (left panel). The indicated percentages (0.15% vs 4.75%) are percentage of MAIT cells (out from CD3⁺ gated cells). FIG. 12B shows expression of CD8a and CD8B of MAIT cells in CB (right) and IVB (left). The indicated numbers are percentage of MAIT cells in IVB or CB. FIG. 12C shows expression of CD45RA and CCR7 (upper panels), and CD45RA and CD62L (lower panels) of MAIT cells in CB (right) and IVB (left). CD45RA⁺CCR7⁺ cells are naïve cells; CD45RA⁻ CCR7⁻ cells are effector memory cells; CD45RA⁺CD62L⁺ cells are naïve cells; CD45RA⁻ CD62L′ cells are effector memory cells. The indicated numbers are percentage of MAIT cells in IVB or CB. FIG. 12D shows expression of CD45RA and CD45RO (upper panel), and CD27 and CD45RO (lower panel) of MAIT cells in CB (right) and IVB (left). CD45RA⁺CD45RO⁻ cells are naïve cells; CD45RA⁻CD45RO⁺ cells are activated or memory cells; CD27⁺CD45RO⁺ cells are central memory cells; CD27⁻CD45RO⁺ cells are effector memory cells. The indicated numbers are percentages of MAIT cells in IVB or CB.

FIGS. 13A-13B shows flow cytometry of engineered MAIT cells. FIG. 13A shows the gating strategy for flow cytometry starting from singlets (top) to live cells (middle left) to MAIT cells (defined by CD161⁺TCRα7.2⁺, middle right) to MAIT NYESO1-TCR (Vβ13.1+) cells (bottom). FIG. 13B shows flow cytometry for MAIT cells after separation with TCRα7.2 and transduction with NY-ESO-1 TCR for a representative placental derived donor. Populations before separation and after positive bead separation are presented.

FIG. 14 shows cell numbers of MAIT cells (CD161⁺TCRα7.2⁺) at days 0, 4, 7 and 10 following activation from two separate placentas.

FIGS. 15A-15E show expression of the cytokines IFNγ (FIG. 15A), IL2 (FIG. 15B), TNFα (FIG. 15C) as measured by intracellular staining and flow cytometry following co-culture of MAIT NYESO1 TCR cells or non-transduced controls with target cells expressing NYESO-1 for 6 hours at a 1:1 effector to target ratio. Results represent 2 different placenta-derived MAIT cells. FIG. 15D shows representative plots of MAIT NY-ESO-1 TCR cells or non-transduced controls co-cultured with T2-ESO or T2-HIV targets cells and stained with antibodies for IFNγ, IL2 and TNFα. FIG. 15E shows categorization of MAIT NYESO-1 TCR cells by expression of either 1, 2 or 3 cytokines (IFNγ, IL2 and TNFα) as a representation of their poly-functional state. MAIT 1 and MAIT 2 represent MAIT populations from two distinct donors.

FIG. 16 depicts the results of an ELISA assay for IFNγ secretion following co-culture of 24 hours between MAIT-NYESO1-TCR cells and target cells at a 1:1 ratio. MAIT 1 and MAIT 2 represent MAIT populations from two distinct donors.

FIGS. 17A-17F show expression of activation markers CD137 (FIGS. 17A-17B), CD69 (FIGS. 17C-17D), CD25 (FIGS. 17E-17F) as measured by flow cytometry following co-culture of MAIT NYESO1 TCR cells or non-transduced control with target cells expressing NYESO-1 for 24 hours at a 1:1 effector to target ratio. Graphs represent 2 different MAIT cells (designated MAIT 1 and MAIT 2) co-cultured with A375, M624, 526, T2-ESO or T2-HIV targets cells. Flow cytometry plots depict representative MAIT cells co-cultured with T2 target cells. FIG. 17A—CD137 expression (% TCR⁺CD137⁺ cells). FIG. 17B—flow cytometry plots of CD137 expression. FIG. 17C—CD69 expression (% TCR⁺CD69⁺ cells). FIG. 17D—flow cytometry plots of CD69 expression. FIG. 17E—CD25 expression (% TCR⁺CD25⁺ cells). FIG. 17F—flow cytometry plots of CD25 expression. FIGS. 18A-18B depicts cytotoxic activity of engineered MAIT cells. —FIG. 18A killing assay measured by expression of active caspase-3 by flow cytometry following co-culture of MAIT cells expressing NY-ESO-1 TCR or non-transduced (NT) controls with target cells at a 1:1 ratio for 1.5 hours. FIG. 18B-Representative plots of flow cytometry with caspase-3 staining depicting targeted killing of T2-ESO cells by MAIT 1 NYESO1-TCR cells.

FIGS. 19A-19B show expression of CD107 as measured by antibody staining and flow cytometry following co-culture of MAIT NYESO1 TCR cells or non-transduced controls with target cells expressing NYESO-1 for 6 hours at a 1:1 effector to target ratio. FIG. 19A is a quantification of the percentile of TCR⁺CD107⁺ cells. FIG. 19B provides representative plots of MAIT NY-ESO-1 TCR cells or non-transduced controls co-cultured with T2-ESO or T2-HIV targets cells and stained with antibodies for CD107.

FIG. 20 quantifies the results of an ELISA experiment for granzyme B (GZMB) secretion following co-culture of 24 hours between MAIT-NYESO1-TCR cells and target cells at a 1:1 ratio.

### DETAILED DESCRIPTION OF THE INVENTION

The present disclosure relates in general to the field of genetic engineering of immune cells, specifically to mucosal-associated invariant T (MAIT) cells genetically engineered to express an exogeneous T cell receptor (TCR) and uses thereof. More specifically, the invention in embodiments thereof relates to cell compositions adapted for adoptive transfer cell therapy (ACT) providing for improved therapeutic modalities. In various embodiments, compositions and methods in accordance with the invention may be used in the treatment of cancer and other conditions condition associated with expression of an HLA class I-restricted antigen.

In some embodiments, the invention relates to compositions and methods utilizing placental MAIT cells, in particular MAIT cells derived from placental intervillous blood (IVB), engineered to express an exogeneous TCR. In some embodiments, the exogeneous TCR is specific to a tumor antigen, for example a cancer testis antigen such as NY-ESO-1 and/or LAGE-1A.

In one aspect, there is provided a cell composition comprising a population of engineered MAIT cells expressing an exogenous TCR, wherein the MAIT cells are derived from placenta, optionally further comprising a pharmaceutically acceptable carrier.

In another aspect, there is provided a cell composition adapted for ACT, the composition comprising a substantially purified population of MAIT cells engineered to express an exogenous TCR, wherein the TCR recognizes a tumor antigen selected from the group consisting of NY-ESO-1 and LAGE-1A.

In another aspect, there is provided a method of treating a subject afflicted with a condition associated with expression of an HLA class I-restricted antigen, comprising administering to the subject a cell composition as defined herein.

In another aspect, the invention provides a method of treating a subject having a tumor or malignancy, comprising administering to said subject a cell composition as defined herein.

In another aspect, there is provided a method of treating a subject afflicted with a condition associated with expression of an HLA class I-restricted antigen, comprising administering to the subject a cell composition comprising a population of engineered MAIT cells expressing an exogenous TCR, and a pharmaceutically acceptable carrier.

In another aspect, the invention provides a process for producing a cell composition adapted for ACT, the process comprising the steps as detailed herein.

These and other aspects are described in greater detail hereinbelow.

### Definitions

Unless otherwise defined, all technical and/or scientific terms used herein have the same meaning as commonly understood by one of ordinary skill in the art to which the engineered mucosal-associated invariant T (MAIT) cells and methods of use thereof pertains. Although methods and materials similar or equivalent to those described herein can be used in the practice or testing of embodiments of the engineered mucosal-associated invariant T (MAIT) cells and methods of use thereof, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

The terms “comprise”, “comprises”, “comprising”, “includes”, “including”, “having” and their conjugates mean “including but not limited to”.

As used herein, the singular form “a”, “an” and “the” include plural references unless the context clearly dictates otherwise. For example, the term “an enzyme” or “at least one enzyme” may include a plurality of enzymes, including mixtures thereof.

Except where indicated otherwise, the term “population”, when used in conjunction with a particular cell attributes or attributes, may encompass a collection of cells, at least 70% of which exhibit the mentioned attribute or attributes. In other embodiments, at least 80%, at least 85%, at least 90%, at least 92%, at least 95%, at least 96%, at least 97%, at least 98%, or at least 99% of the cells in the collection exhibit the mentioned attribute or attributes. In some embodiments, the population is isolated from a cell type distinct from a particular attribute. In some embodiments, a population of engineered MAIT cells described herein comprises endogenous and exogenous attributes. In some embodiments, a population of MAIT cells that have not been engineered to express an exogenous antigen receptor, described herein, comprises endogenous and exogenous attributes.

With reference to placenta-derived cells, except where indicated otherwise, “placenta”, “placental”, and the like, may encompass various solid or liquid portions of the placenta such as decidua parietalis, intervillous blood (IVB), and decidua basalis. Placenta-derived MAIT cells may be obtained, in various embodiments, from either fetal or, in other embodiments, maternal regions of the placenta, or in other embodiments, from both regions. In some embodiments, MAIT cells are obtained from placental blood. In some embodiments, MAIT cells are obtained from fetal or maternal blood sources of the placenta. In some embodiments, MAIT cells are obtained from fetal and maternal blood sources of the placenta. In some embodiments, MAIT cells are obtained from fetal blood sources of the placenta. In some embodiments, MAIT cells are obtained from maternal blood sources of the placenta. In some embodiments, MAIT cells are obtained from intervillous blood of the placenta. In some embodiments, MAIT cells are obtained from decidua parietalis of the placenta. In some embodiments, MAIT cells are obtained from decidua basalis of the placenta. In some embodiments, MAIT cells are not obtained from cord blood.

As used herein, the terms “peripheral blood derived MAIT cells”, “peripheral blood MAIT cells”, or “peripheral MAIT cells” comprise MAIT cells isolated from peripheral blood (PB). These terms may be used interchangeably, having all the same qualities and meanings.

As used herein, the terms “intervillous blood (IVB)-derived MAIT cells” or “IVB MAIT cells” comprise MAIT cells isolated from intervillous blood of placenta. These terms may be used interchangeably, having all the same qualities and meanings.

As used herein, “MAIT cells” would broadly encompass MAIT cells from any source including, but is not limited to peripheral blood or intervillous blood.

In some embodiments of populations of engineered MAIT cells, the MAIT cells are derived from maternal sources (e.g. tissue and/or blood sources) of the placenta. In some embodiments of populations of engineered MAIT cells, the MAIT cells are derived from maternal blood sources of the placenta. In some embodiments of populations of engineered MAIT cells, the MAIT cells are obtained from intervillous blood of the placenta. In some embodiments of populations of engineered MAIT cells, the MAIT cells are not obtained from cord blood. In some embodiments, MAIT cells are derived from a maternal source, a placental source, or a IVB source, or a combination thereof. In some embodiments, MAIT cells are derived from a fetal source, a maternal source, a fetal and maternal source, a placental source, or a IVB source, or a combination thereof; and are not derived from cord blood. In a particular embodiment, MAIT cells are derived from IVB.

Placental cells may be obtained, in various embodiments, from a full-term or pre-term placenta. In a particular embodiment, placental cells are obtained from full-term placenta, for example week 38-39 placentas may conveniently be used. A convenient source of placental tissue is a post-partum placenta (e.g., less than 48 hours after birth); however, a variety of sources of placental tissue or cells may be contemplated by the skilled person. In other embodiments, the placenta is used within 24 hours (in some embodiments, while preserved in physiological buffer), 18 hours, 14 hours, 10 hours, 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. In some embodiments, the donor is 40 years or younger, in other embodiments 35 years old or younger, while in other embodiments, the donor may be any woman of childbearing age.

Methods for isolating lymphoid or MAIT cells from placental intervillous blood (IVB) are generally known in the art. An exemplary, non-limiting protocol utilizes blood that drips from a placenta lifted with the clamped umbilical cord facing down. Such methods were shown to have a very low rate of cross-contamination between IVB and umbilical cord blood. Those skilled in the art are familiar with methods of checking purity of cell populations and, if necessary, enhancing the purity, using cell sorting and the like.

Exogenous antigen receptor, as used herein, comprises an antigen receptor not naturally present on the MAIT cells. Example of such receptors is exogenous TCR. Typically, the MAIT cells disclosed herein are engineered to express an exogenous antigen receptor. In some embodiments, an engineered MAIT cells comprises a TCR-MAIT cell, expressing an exogenous TCR.

In certain embodiments, the exogenous antigen receptor is permanently integrated into the engineered IVB-derived MAIT cells. In other embodiments, the IVB-derived MAIT cells are engineered to transiently express the exogenous antigen receptor. Permanent is used herein to denote insertion of exogenous DNA into the genome of the target cells (which may utilize various viral and non-viral technologies generally known in the art). Transient is used to denote engineering the cells to temporarily express the exogenous antigen receptor gene, in some embodiments via mRNA insertion into the cells.

A skilled artisan would appreciate that the exogenous antigen receptors disclosed herein comprises in certain embodiments, antigen binding domains that result in the MAIT cells comprising these exogenous antigen receptors to bind target molecules, i.e., the antigen of interest. As used herein, the terms “antigen” and “target molecule” may be used interchangeably having all the same qualities and meanings.

The terms “positive” or “high”, “dim” or “low,” or “negative” for any of the cell-surface markers described herein, and all such designations are well accepted terms useful for the practice of the assays and methods described herein. A cell is considered “positive” or “high” for a cell-surface marker if it expresses the marker on its cell-surface in amounts sufficient to be detected using methods known to those of skill in the art, such as contacting a cell with an antibody that binds specifically to that marker, and subsequently performing flow cytometric analysis of such a contacted cell to determine whether the antibody is bound the cell. It is to be understood that while a cell may express messenger RNA for a cell-surface marker, in order to be considered positive for the assays and methods described herein, the cell must express the cell surface marker of interest on its surface. A cell is considered “dim” or “low” for a cell-surface marker if it expresses the marker on its cell-surface in amounts sufficient to be detected using methods known to those of skill in the art, such as contacting a cell with an antibody that binds specifically to that marker, and subsequently performing flow cytometric analysis of such a contacted cell to determine whether the antibody is bound the cell, but there exists another distinct population of cells that expresses the marker at a higher level, giving rise to at least two populations that are distinguishable when analyzed using, for example, flow cytometry (e.g. cells designated “high” or “bright” represent the highest expression level of said marker in said population). Similarly, a cell is considered “negative” for a cell-surface marker if it does not express the marker on its surface in amounts sufficient to be detected using methods known to those of skill in the art, such as contacting a cell with an antibody that binds specifically to that marker and subsequently performing flow cytometric analysis of such a contacted cell to determine whether the antibody is bound the cell.

“Cell sorting”, as used herein, encompasses typically immunological-based methods of positive and negative selection, which result in the physical isolation of a cell type, having a specific cell surface marker or combination of markers using an antibody or an antibody fragment, or a combination of antibodies or antibody fragments, which specifically recognize(s) the marker(s). Examples include, but are not limited to cell sorting by fluorescence-activated cell sorting (FACS), magnetic beads, magnetic-activated cell sorting (MACS), columns-based cell sorting, and immuno-panning.

In some embodiments, the present disclosure provides a population of engineered mucosal-associated invariant T (MAIT) cells comprising an exogenous antigen receptor, wherein the MAIT cells are derived from placenta. In some embodiments, the engineered MAIT cells are derived from placental intervillous blood (IVB). The exogenous antigen receptor of these engineered MAIT cells can be a T cell receptor (TCR). In some embodiments, the TCR recognizes a tumor antigen.

### Mucosal-Associated Invariant T (MAIT) Cells

Mucosal associated invariant T (MAIT) cells, as used herein, comprise T cells that express a semi-invariant TCR, e.g., Vα7.2-Jα33 in humans; in some embodiments associated with the β-chains Vβ2/Vβ13. In other embodiments, the aforementioned alpha chain is associated with a beta chain from the TRBV6 or TRBV20 gene families. In some embodiments, the MAIT cells recognize antigen restricted to non-peptide molecules presented in the context of (non-polymorphic) major histocompatibility complex (MHC) class I-like protein MR1. In some embodiments, the engineered MAIT cells disclosed herein are detectable by staining with MR1-Ag tetramer, e.g., loaded with 5-(2-oxopropylideneamino)-6-D-ribitylaminouracil (5-OP-RU); 5-(2-oxoethylideneamino)-6-D-ribitylaminouracil (5-OE-RU); RL-6,7-diMe (PubChem CID 168989); RL-6-Me-7-OH (PubChem CID 440869), or diclofenac (PubChem CID 3033). In other embodiments, one of the following compounds may also be used: 6-(1H-indol-3-yl)-7-hydroxy-8-ribityllumazine or photolumazine III (PLIII); 6-(2-carboxyethyl)-7-hydroxy-8-ribityllumazine or photolumazine I; 5-Hydroxydiclofenac (PubChem CID 3052566); 4-Hydroxydiclofenac (PubChem CID 116545); benzbromarone (PubChem CID 2333); chloroxine (PubChem CID 2722); floxuridine (PubChem CID 5790); galangin (4H-1-benzopyran-4-one,3,5,7-trihydroxy-2-phenyl or 3,5,7-trihydroxyflavone); or mercaptopurine (PubChem CID 667490) (see, e.g., Corbett et al., Antigen Recognition by MR1-Reactive T Cells; MAIT Cells, Metabolites, and Remaining Mysteries. Front Immunol. 11:1961 (2020), and the references cited therein).

Conveniently, MAIT cells may be referred to in some embodiments as TCRVα7.2⁺CD161⁺ or more typically TCRVα7.2⁺ CD161^{high}.

In certain embodiments, the MAIT cells disclosed herein are human MAIT cells. In some embodiments, MAIT cells disclosed herein are allogeneic with respect to the recipient of a population of engineered MAIT cells, as described herein.

Methods for isolating and characterizing MAIT cells and other leukocyte sub-populations are known in the art. Solely for exemplification, leukocyte sub-populations can be isolated and/or analyzed using the gating strategy as described herein.

There are quantitative and qualitative differences between cord blood and adult blood-derived MAIT cells (see e.g., Youssef et al., Ontogeny of Human Mucosal-Associated Invariant T Cells and Related T Cell Subsets. JEM 215:459-479 (2018)). Vα7.2 and CD161 staining allows identification of circulating stage 3 MAIT cells at birth. However, the Vα7.2⁺ CD161^{high} fraction in cord blood may also encompass other T cells probably sharing a common developmental pathway. In cord blood, MAIT cells are of premature state and they possess naïve phenotype (CD45RA⁺/RO⁻) and express the CD8αβ heterodimer, whereas MAIT cells in adults are mature, mostly exhibit memory phenotype and express the CD8αα homodimer. The very low expansion of Vα7.2⁺ CD161^{high} T cells after birth may be related to cell intrinsic characteristics or to limited availability of microbial-derived MR1-ligands. Naïve cord blood Vα7.2⁺ CD161^{high} T cells expressed significantly lower levels of PLZF than adult MAIT cells, suggesting that final maturation of cord blood Vα7.2⁺ CD161^{high} T cells requires an early activation signal after birth. Cord blood Vα7.2⁺ CD161^{high} T cells proliferate strongly after stimulation by phytohemagglutinin (PHA), similar to conventional CD8 T cells, whereas adult Vα7.2⁺ CD161^{high} T cells proliferate much less efficiently. In contrast to mature MAIT cells in adult blood, cord blood Vα7.2⁺CD161^{high} T cells are not able to display immediate effector functions toward bacterially infected cells. These data indicate that, although they have intrinsic ability to proliferate, cord blood Vα7.2⁺ CD161^{high} T cells need functional maturation and/or expansion after birth to acquire detectable effector activities after recognition of a microbe-derived antigen.

Since cord blood Vα7.2⁺ CD161^{high} cells exhibit a naïve phenotype and intermediate PLZF levels, they are unable to rapidly produce cytokines or cytotoxic molecules in response to bacterial ligands, in contrast to mature adult MAIT cells. Neither do cord blood MAIT cells respond to stimulation by exogenous IL-12 and IL-18, despite high expression of the receptors for these cytokines. As further shown in Chen et al., Circulating Mucosal-Associated Invariant T Cells in a Large Cohort of Healthy Chinese Individuals From Newborn to Elderly. Front. Immunol., vol. 10, article 260 (2019), cord blood MAIT cells have naïve phenotype and do not secrete IFN-γ, IL17A, and TNF-α following in vitro stimulation with PMA/ionomycin. Taken together, these data indicate that cord blood premature MAIT cells are phenotypically and functionally different from mature MAIT cells derived from adult subjects.

In some embodiments, MAIT cells disclosed herein comprise CD161⁺Vα7.2⁺, CD4⁻CD3⁺ lymphocytes, which, in further embodiments, also bind to MR1-Ag tetramer. In various embodiments, at least 50% of the cells in the population are CD161⁺Vα7.2⁺, CD4⁻CD3⁺. In some embodiments, at least 60%, at least 70%, at least 80%, at least 85%, at least 90%, at least 95%, at least 97%, at least 98%, or at least 99% of the cells in the population are CD161⁺Vα7.2⁺, CD4⁻CD3⁺.

In yet some embodiments, MAIT subsets are utilized, including but not limited to CD8⁺ cells (more specific embodiments of which are CD8⁺CD4-cells), CD8⁻CD4⁻ cells, or CD4⁺ cells. In various embodiments, at least 50% of the cells in the population are CD8⁺. In other embodiments, at least 60%, at least 70%, at least 80%, at least 85%, at least 90%, at least 95%, at least 97%, at least 98%, or at least 99% of the cells in the population are CD8⁺. In various embodiments, at least 50% of the cells in the population are CD8⁺CD4⁻. In other embodiments, at least 60%, at least 70%, at least 80%, at least 85%, at least 90%, at least 95%, at least 97%, at least 98%, or at least 99% of the cells in the population are CD8⁺CD4⁻. In various embodiments, at least 50% of the cells in the population are CD8 CD4⁻. In other embodiments, at least 60%, at least 70%, at least 80%, at least 85%, at least 90%, at least 95%, at least 97%, at least 98%, or at least 99% of the cells in the population are CD8 CD4⁻. In various other embodiments, no more than 50% of the cells in the population are CD8 CD4⁻. In various other embodiments, no more than 40%, 30%, 20%, 10%, 5%, 3%, 2% or 1% of the cells in the population are CD8⁻CD4⁻. In various other embodiments, no more than 50% of the cells in the population are CD4⁺. In various other embodiments, no more than 40%, 30%, 20%, 10%, 5%, 3%, 2% or 1% of the cells in the population are CD4⁺.

In certain embodiments, the MAIT cells disclosed herein are additionally CD62L^{lo}CD122^{int}CD127^{hi}CD95^{hi}, which in some embodiments indicates an effector memory phenotype. In other embodiments, the MAIT cells disclosed herein are CD45RA⁻CCR7⁻, also reflecting, in some embodiments, an effector memory phenotype. In other embodiments, CD45RO⁺ is an additional characteristic of effector memory cells. In various embodiments, at least 50% of the cells in the population are CD62L^{lo}CD122^{int}CD127^{hi}CD95^{hi}, in other embodiments, at least 60%, at least 70%, at least 80%, at least 85%, at least 90%, at least 95%, at least 97%, at least 98%, or at least 99% of the cells in the population are CD62L^{lo}CD122^{int}CD127^{hi}CD95^{hi}. In yet other embodiments, at least 5%, 10%, 20%, 30% or 40% of the cells in the population are CD62L^{lo}CD122^{int}CD127^{hi}CD95^{hi}. In various embodiments, at least 50% of the cells in the population are CD45RA⁻CCR7⁻; in other embodiments, at least 60%, at least 70%, at least 80%, at least 85%, at least 90%, at least 95%, at least 97%, at least 98%, or at least 99% of the cells in the population are CD45RA⁻CCR7⁻. In yet other embodiments, at least 5%, 10%, 20%, 30% or 40% of the cells in the population are CD45RA⁻CCR7⁻. In yet other embodiments, no more than 40%, 30% 20%, 10%, or 5%, of the cells in the population are CD45RA⁻ CCR7⁻. In various embodiments, at least 50% of the cells in the population are CD45RO⁺; in other embodiments, at least 60%, at least 70%, at least 80%, at least 85%, at least 90%, at least 95%, at least 97%, at least 98%, or at least 99% of the cells in the population are CD45RO⁺. In yet other embodiments, at least 5%, 10%, 20%, 30% or 40% of the cells in the population are CD45RO⁺.

In other embodiments, the MAIT cells disclosed herein are CD45RA⁻ CCR7⁺, reflecting, in some embodiments, a central memory phenotype. In other embodiments, CD45RO⁺ is an additional characteristic of central memory cells. Unless indicated otherwise, “central memory” T cells encompasses transitional memory cells (CCR7⁻CD45RO⁺CD28⁺CD95⁺). In various embodiments, at least 50% of the cells in the population are CD45RA⁻CCR7⁺; in other embodiments, at least 60%, at least 70%, at least 80%, at least 85%, at least 90%, at least 95%, at least 97%, at least 98%, or at least 99% of the cells in the population are CD45RA⁻CCR7⁺. In various embodiments, at least 50% of the cells in the population are CD45RO⁺; in other embodiments, at least 60%, at least 70%, at least 80%, at least 85%, at least 90%, at least 95%, at least 97%, at least 98%, or at least 99% of the cells in the population are CD45RO⁺. In various embodiments, at least 50% of the cells in the population are CCR7⁻CD45RO⁺CD28⁺CD95⁺; in other embodiments, at least 60%, at least 70%, at least 80%, at least 85%, at least 90%, at least 95%, at least 97%, at least 98%, or at least 99% of the cells in the population are CCR7⁻CD45RO⁺CD28⁺CD95+. In various other embodiments, no more than 50% of the cells in the population are CD45RA⁻ CCR7⁺.

In other embodiments, the MAIT cells disclosed herein are CD45RA⁺CCR7⁻, reflecting, in some embodiments, a terminally differentiated (terminal effector) phenotype. In various embodiments, at least 50% of the cells in the population are CD45RA⁺CCR7⁻; in other embodiments, at least 60%, at least 70%, at least 80%, at least 85%, at least 90%, at least 95%, at least 97%, at least 98%, or at least 99% of the cells in the population are CD45RA⁺CCR7⁻. In various other embodiments, no more than 50% of the cells in the population are CD45RA⁺CCR7⁻. In various other embodiments, no more than 40%, 30%, 20%, 10%, 5%, 3%, 2%, 1%, 0.5% or 0.2% of the cells in the population are CD45RA⁺CCR7⁻.

In other embodiments, the MAIT cells disclosed herein express interleukin (IL)-18Rα, CD127, α4β7, and/or PD-1. In some embodiments, the MAIT cells express IL-18Rα, CD127, and α4β7. In some embodiments, the MAIT cells express PD-1. In some embodiments, the MAIT cells express IL-18Rα, CD127, α4β7, and PD-1. In some embodiments, the cells also express the transcription factors promyelocytic leukemia zinc finger (PLZF), RORγt, Helios, Eomesodermin (Eomes), and/or T-box transcription factor (T-bet). In some embodiments, the MAIT cells express the transcription factors PLZF, RORγt, Helios, Eomesodermin. In some embodiments, the MAIT cells express T-box transcription factor. In some embodiments, the MAIT cells express the transcription factors PLZF, RORγt, Helios, Eomesodermin, and T-box transcription factor. Alternatively or in addition, the cells express the surface markers CD26, CD44, CD69, or CD25; or the receptors interleukin 7 receptor (IL-7R, also known as CD127), IL-12R, IL-15R, or IL-18R. In some embodiments, the cells express Inducible T-cell costimulator (ICOS). Each of the above proteins, and each combination thereof, represents a separate embodiment. In some embodiments, MAIT cells to be used in accordance with the invention are TCRVa7.1⁺CD26⁺.

In some embodiments, the MAIT cells disclosed herein express genes related to tissue repair (e.g., Transforming Growth Factor Beta-1, Platelet Derived Growth Factor Subunit B, or Matrix Metallopeptidase) or angiogenesis (e.g., Granulocyte-Macrophage Colony-Stimulating Factor, Vascular Endothelial Growth Factor, or Hypoxia Inducible Factor 1 Subunit Alpha) upon stimulation with 5-OP-RU.

In certain embodiments, the engineered MAIT cells disclosed herein are capable of reacting to antigens restricted by MHC class-I-related protein MR1 (i.e., can recognize and react to cells presenting a MAIT cell antigen in the context of MR1). In other embodiments, the engineered MAIT cells recognize microbial-derived riboflavin precursor derivatives. In some embodiments, the engineered MAIT cells secrete inflammatory cytokines (e.g., interferon-gamma [IFN-g or IFN-γ], tumor necrosis factor alpha, interleukin 17, or colony stimulating factor 2 [CSF2/GM-CSF]) upon activation, e.g., by recognition of MR-1 ligands or, in other embodiments, in an MR1-independent manner. In other embodiments, IL-17A, TNF-a, CSF2 or MIP-1 are all secreted. In other embodiments, IL-26, oncostatin M (OSM), or heparin binding early growth factor (HBEGF) are upregulated upon stimulation with IL-12, IL-18, IL-15, or Tumor necrosis factor-like protein 1A (TL1A). Alternatively or in addition, the engineered MAIT cells disclosed herein perform perforin/granzyme and/or granulysin dependent cytotoxicity of target cells upon activation. In other embodiments, IFN-γ, perforin, granulysin or granzyme B are all upregulated. In another embodiment IFN-γ, perforin and granzyme B are up-regulated.

In some embodiments, levels of a particular subtype mentioned herein, including but not limited to CD8⁺ cells, are enriched in the population of engineered MAIT cells disclosed herein at least 1.5-fold, at least 2-fold, at least 3-fold, at least 5-fold, or at least 10-fold, compared with peripheral blood (PB)-derived MAIT cells, or, in another embodiments, compared with liver-derived MAIT cells. In other embodiments, levels of a particular subtype mentioned herein, including but not limited to CD8 CD4⁻ cells, are enriched in the population of engineered MAIT cells disclosed herein at least 1.5-fold, at least 2-fold, at least 3-fold, at least 5-fold, or at least 10-fold, compared with peripheral blood (PB)-derived MAIT cells, or, in another embodiments, compared with liver-derived MAIT cells. In other embodiments, levels of a particular subtype mentioned herein, including but not limited to CD4+ cells, are enriched in the population of engineered MAIT cells disclosed herein at least 1.5-fold, at least 2-fold, at least 3-fold, at least 5-fold, or at least 10-fold, compared with peripheral blood (PB)-derived MAIT cells, or, in another embodiments, compared with liver-derived MAIT cells.

In certain embodiments, the methods described herein comprise expansion and/or enrichment of the engineered MAIT cells in vitro before administration to a subject. In various embodiments, expansion or enrichment is performed before and/or after engineering the MAIT cell to express the exogenous antigen receptor. According to an advantageous embodiment, processes of the invention include an enrichment step performed prior to engineering and an expansion step performed following engineering. In other embodiments, activation or stimulation assays are performed to characterize or determine the quality of the engineered MAIT cells. It will be appreciated that protocols for expanding and activating T cells in vitro often may overlap. Typically, however, activation requires stimulation of antigen receptors and coreceptors, in combination with cytokine treatment. Once cells are activated, they can, in some embodiments, be further expanded (without reverting to naïve status) using cytokines alone.

In some embodiments, MAIT cells are ex-vivo expanded for at least about 5 days, in other embodiments 5-10 days; in other embodiments at least 10 days; in other embodiments 10-15 days; in other embodiments at least 15 days; in other embodiments 15-20 days; and in some embodiments, at least 20 days. For example, MAIT cells are ex-vivo expanded for 7-12 days, e.g. for 10 days.

Throughout this application, various embodiments may be presented in a range format. It should be understood that the description in range format is merely for convenience and brevity and should not be construed as an inflexible limitation on the scope of the engineered MAIT cells and uses thereof. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

Methods for stimulating MAIT cells are known in the art. In one non-limiting embodiment, Hela cells overexpressing the human MR1 protein (Hela-hMR1) are washed and incubated with Escherichia coli, Dh5α ATCC strain (typically at a multiplicity of infection of 10-100 bacteria per HeLa cell), in antibiotic-free DMEM, for 30 min at 37° C., washed, then incubated at 37° C. for 2 hours in complete medium with 100 μg/mL gentamicin and 10 μg/mL chloramphenicol. MAIT cells are added for an overnight co-culture, then cells are harvested and stained for FACS analysis.

Additional methods for expanding and/or enriching MAIT cells are known in the art. In a non-limiting embodiment, MAIT cells are incubated with 5 μg/mL CpG, 300 nM 5-(2-oxopropylideneamino)-6-D-ribitylaminouracil (5OP-RU), and 50 ng/mL human IL-15. In certain embodiments, IL-15 is included to preferentially enhance the expansion of memory T cells. In some embodiments, IL-15 (e.g. 10-200 ng/ml) and 5-OP-RU (e.g. 100-1000 nM) are conveniently used.

In some embodiments, MAIT cells are activated in vitro by CD3/CD28 stimulation (e.g., using functionalized beads or polymers, available commercially under the trademark TransAct™) in the presence of autologous or allogeneic irradiated PBMCs and IL-2, IL-7, IL-12, IL-18, IL-21, IL-15, or analogues thereof, or combinations thereof. In other embodiments, CD3/CD28 beads (commercially available at clinical grade under the trademark ClinEx Vivo™ Dynabeads®) are used with IL-7 and IL-2. A non-limiting exemplary protocol includes incubation for 10-14 days with ClinEx Vivo™ Dynabeads® at a cell:bead ratio of 3:1 in X Vivo-15™ (BioWhittaker®, Walkersville, MD), 100 units/mL IL-2, and 10 ng/ml of IL-7. Once activated, MAIT cells can be further expanded in the presence of cytokines, in various embodiments either with or without the aforementioned ligands. According to exemplary embodiments, such expansion methods may be employed following engineering of the cells to express the exogeneous TCR.

In other embodiments, MAIT cells are activated in vitro in the presence of MAIT cell activating ligands, such as 5OP-RU, 5-amino-4-D-ribitylaminouracil dihydrochloride (5-ARU), 5-(2-oxoethylideneamino)-6-D-ribitylaminouracil (5-OE-RU), 5-amino-6-ribitylamino-2,4-(1H, 3H)-pyrimidinedione (5-A-RU), or other Riboflavin (vitamin B2)-derivatives. In certain embodiments, the ligand(s) are provided in combination with cytokines, e.g., the cytokines mentioned herein. As a non-limiting example, MAIT cells can be expanded in 100 nM 5-OP-RU and 100 IU/mL IL-2, e.g., for 6-17 days. In other embodiments, MAIT cells are expanded and/or activated in vitro in the presence of MAIT cell activating drug metabolites (e.g., diclofenac metabolites). Once activated, MAIT cells can be further expanded in the presence of cytokines, in various embodiments either with or without the aforementioned ligands.

In some embodiments, MAIT cells are expanded and/or activated in vitro in the presence of IL-12 (2 ng/mL), IL-18 (50 ng/mL), and 10 mM (millimolar) 5-OP-RU, or IL-12+IL-18 or IL-15+IL-18 as described in the art.

In some embodiments, the MAIT cells to be engineered produce IL-17 and upregulate the Th17-associated transcription factor RORC (RORγt) upon PMA and ionomycin stimulation, but not upon CD3+CD28 stimulation.

In some embodiments, the engineered MAIT cells disclosed herein produce IFN-γ and upregulate T-bet upon PMA/ionomycin stimulation. In more specific embodiments, these cells are CD8⁺.

In various embodiments, over 50%, over 60%, or over 70% of the population of engineered MAIT cells disclosed herein express CD69. Alternatively, or in addition, under 30%, under 25%, or under 20% of the population of engineered MAIT cells express Ki67.

In other embodiments, over 50%, over 60%, or over 70% of the population of engineered MAIT cells disclosed herein express PD-1. In other embodiments, over 40%, over 50%, or over 60% of the population of engineered MAIT cells express CD38. In other embodiments, over 10%, over 15%, or over 20% of the population of engineered MAIT cells express CD25. In other embodiments, any combination of 2 or all 3 of the above markers are expressed, whose percentages may be freely combined with one another. In some embodiments, over 50%, over 60%, over 70%, or over 80% of the engineered MAIT cells are PD-1/LAG-3″, which is indicative of cells not exhibiting T cell exhaustion. In other embodiments, CTLA-4, TIGIT, 2B4, BTLA, CD57, TIM-3, or KLRG-1 are used to detect T-cells exhibiting exhaustion. In certain embodiments, such cells are removed or depleted from the T cell population.

In certain embodiments, the MAIT cells do not appreciably proliferate in mixed lymphocyte reactions (MLR) when incubated with allogeneic cells. Methods for measuring proliferation of lymphoid cells in response to alloantigen stimulation are known in the art.

In some embodiments, the MAIT cells disclosed herein (e.g. MAIT cells used to produce cell compositions in accordance with the invention) express CCR2, CCR5, CCR6, CCR9, CXCR4, CXCR3, VLA-4, or CXCR6, or a combination of 2, 3, 4, 5, 6, 7 or all 8 of these receptors. Alternatively, or in addition, the MAIT cells express activating receptors, e.g., NKG2D, NKp30, NKp44 or NKG2D and NKp30 and NKp44. In some embodiments, the MAIT cells express high levels of CXCR4 and moderate levels of CCR9, but low or no expression of CXCR2. Alternatively, or in addition, the MAIT cells also express CXCR3. In some embodiments, the MAIT cells disclosed herein express one or more cytokine receptors such as IL-7R, IL-12R, IL-15R, IL-18R, and IL-21R, or any combination thereof.

In some embodiments, the engineered MAIT cells disclosed herein secrete IFN-γ at levels at least 1.2-fold, at least 1.5-fold, at least 1.8-fold, at least 2-fold, at least 2.5-fold, or at least 3-fold higher than an equivalent number of the peripheral blood (PB)-derived MAIT cells, when exposed to cells expressing target antigen, e.g., in response to riboflavin-producing Escherichia coli. In some embodiments, liver-derived MAIT cells are used instead of PB-derived MAIT cells as the basis of comparison.

In some embodiments, the engineered MAIT cells disclosed herein release granzyme at levels at least 1.2-fold, at least 1.5-fold, at least 1.8-fold, at least 2-fold, at least 2.5-fold, or at least 3-fold higher than an equivalent number of the PB-derived MAIT cells, when exposed to cells expressing target antigen, e.g., in response to riboflavin-producing E. coli. In other embodiments, liver-derived MAIT cells are used instead of PB-derived MAIT cells as the basis of comparison.

In some embodiments, the engineered MAIT cells disclosed herein mediate perforin/granzyme dependent cytotoxicity of target cells (for example, as measured by ELISPOT-detection of granzyme B) at levels at least 1.2-fold, at least 1.5-fold, at least 1.8-fold, at least 2-fold, at least 2.5-fold, or at least 3-fold higher than the PB-derived MAIT cells, when exposed to cells expressing target antigen, e.g., in response to riboflavin-producing E. coli. In other embodiments, liver-derived MAIT cells are used instead of PB-derived MAIT cells as the basis of comparison. Cytotoxicity is measured, in some embodiments, by measuring the number of target cells killed when incubated with limiting dilutions of MAIT cells.

In some embodiments, the engineered MAIT cells disclosed herein secrete XCL1, CCL3, CCL4, and CXCL16. In some embodiments, the engineered MAIT cells disclosed herein secrete at least 1 of XCL1, CCL3, CCL4, and CXCL16. In some embodiments, the engineered MAIT cells disclosed herein secrete at least 2 of, or in other embodiments at least 3 of, XCL1, CCL3, CCL4, and CXCL16.

In some embodiments, the engineered MAIT cells disclosed herein secrete at least 1 of, in other embodiments at least 2 of, in other embodiments at least 3 of, or in other embodiments all 4 of XCL1, CCL3, CCL4, and CXCL16 (each of which represents a separate embodiment) at levels at least 1.2-fold, at least 1.5-fold, at least 1.8-fold, at least 2-fold, at least 2.5-fold, or at least 3-fold higher than PB-derived MAIT cells. In some embodiments, the levels of 1 of these cytokines, in other embodiments 2 of these cytokines, in other embodiments 3 of these cytokines, or in other embodiments all 4 of XCL1, CCL3, CCL4, and CXCL16 are at least 1.2-fold, at least 1.5-fold, at least 1.8-fold, at least 2-fold, at least 2.5-fold, or at least 3-fold higher than the PB-derived MAIT cells, while the levels of the other listed cytokine(s) are not appreciably lower than the PB-derived MAIT cells. In other embodiments, liver-derived MAIT cells are used instead of PB-derived MAIT cells as the basis of comparison.

In some embodiments, the engineered MAIT cells disclosed herein secrete/release IL-17 at levels at least 1.2-fold, at least 1.5-fold, at least 1.8-fold, at least 2-fold, at least 2.5-fold, or at least 3-fold higher than the PB-derived MAIT cells, when exposed to cells expressing target antigen, e.g., with co-incubated with MR1-expressing THP-1 cells infected with Pseudomonas aeruginosa. In other embodiments, the engineered MAIT cells disclosed herein secrete/release IL-22 at levels at least 1.2-fold, at least 1.5-fold, at least 1.8-fold, at least 2-fold, at least 2.5-fold, or at least 3-fold higher than the PB-derived MAIT cells, when exposed to cells expressing the target antigen. In other embodiments, the engineered MAIT cells disclosed herein secrete/release IL-17 and IL-22 at levels at least 1.2-fold, at least 1.5-fold, at least 1.8-fold, at least 2-fold, at least 2.5-fold, or at least 3-fold higher than the PB-derived MAIT cells, when exposed to cells expressing the target antigen. In other embodiments, liver-derived MAIT cells are used instead of PB-derived MAIT cells as the basis of comparison.

In some embodiments, the engineered MAIT cells disclosed herein exhibit antigen-independent activation in response to IL-18, particularly in combination with IL-12, IL-15 and/or type I interferons (e.g., interferon-α/β). In some embodiments, the activation is manifest, inter alia, by secretion and upregulated expression of IFN-γ and granzyme B, respectively. In other embodiments, activation is manifest by upregulated expression of the IL-18 receptor (IL-18R).

In some embodiments, the engineered MAIT cells disclosed herein exhibit antigen-independent activation in response to IFN-α/β in combination with IL-12 and IL-18. In some embodiments, the activation is manifest, inter alia, by secretion upregulated expression of IFN-γ and granzyme B, respectively.

Exogenous T Cell Receptor (eTCR)

In some embodiments, the IVB-derived MAIT cells are engineered to express an exogenous antigen receptor, which is an exogenous unmodified or modified T cell receptor (TCR). Such cells comprise “TCR-engineered” MAIT cells. Generating TCR-engineered MAIT cells that express exogenous TCR can be accomplished by various methods generally known in the art. Described below are some methods for illustrative purposes. As used herein, the terms “TCR-MAIT” and “TCR-MAIT cells” encompass MAIT cells (e.g. IVB-derived MAIT cells) that have been engineered to express an exogenous TCR, and may be used interchangeably, having all the same qualities and meaning.

Non-limiting examples of TCR-engineered cells are described in Debets et al., TCR-engineered T cells to treat tumors: Seeing but not touching? Semin Immunol. 28:10-21 (2016); Ping et al., T-cell receptor-engineered T cells for cancer treatment: current status and future directions. Protein Cell. 9:254-266 (2018), and the references cited therein; and in US Pat. Appl. Pub. Nos. 2020/0237820 and 2017/0224733, which are incorporated herein by reference.

As used herein, unmodified TCR comprises TCR isolated from non-MAIT T cells and introduced to e.g. placental or IVB-derived MAIT cells without any modifications for improving expression level, binding affinity, or other features. As used herein, modified TCR comprises TCR isolated from non-MAIT T cells, modified as for example described herein and introduced to e.g. placental or IVB-derived MAIT cells. Non-limiting examples of methods for modifying TCR, include but are not limited to, improving TCR affinity, as described in Ohta et al., Improving TCR affinity on 293T cells. J Immunol Methods 466:1-8 (2019). In certain embodiments, the TCR has a target affinity greater than a K_{D} of 2.5 nanomolar (reflected by a number smaller than 2.5 nM). In other embodiments, the affinity of the TCR for antigen-bound MHC is between 10⁻⁴-10⁻⁶ M.

In some other embodiments, the two TCR protein chains are stabilized through the introduction of a disulfide bond between the two constant domains. In further embodiments, the affinity of the modified TCR for antigen-bound MHC is between 10⁻¹⁰-10⁻¹² M.

TCRs are disulfide-linked membrane-bound heterodimeric proteins expressed on the surface of T cells, which are members of the immunoglobulin superfamily. TCRs engage, via their variable regions, antigenic peptide in complex with the MHC/HLA, to induce downstream T cell signaling (further referred to herein as TCR-mediated signaling). TCRs typically comprise the highly variable alpha and beta chains, which complex with invariant CD3 chain molecules; a minority of TCRs comprise variable gamma and delta chains. Each of the alpha (a) chain and the beta (B) chain comprises two extracellular domains: a variable region (VR) and a constant region (CR). Each variable region (e.g. in the α chain and the β chain) contains three hypervariable regions, also referred to as “complementarity determining regions” (CDRs) separated by framework regions (FRs). CDR3 is the main CDR responsible for antigen binding. The α and β chains also contain joining (J) regions. The β chain also usually contains a diversity (D) region between the V and J regions; however, this D region may be considered part of the J region.

### Target Antigens

In certain embodiments, an exogenous antigen receptor, for example a TCR as described herein, directs the engineered MAIT cells to recognize a tumor antigen.

In certain embodiments, the TCR target comprises a tumor-associated antigen (TAA) or cancer antigen. The term “tumor-associated antigens”, “tumor antigen” or “cancer antigen”, as used herein, refers to any protein, peptide or antigen associated with (carried by, expressed by, produced by, secreted by, etc.) a tumor or tumor cell(s). Tumor-associated antigens may be (nearly) exclusively associated with a tumor or tumor cell(s) and not with healthy normal cells or may be over-expressed (e.g., 50 times, 100 times, 1000 times or more) in a tumor tissue or tumor cell(s) compared to healthy normal tissue or cells. It will be understood by those skilled in the art that recognition of such antigens is typically MHC-dependent, in the case of exogenous TCR. More particularly, a TAA to be targeted by a TCR in accordance with the invention is an antigen capable of being presented (in processed form) by MHC determinants of the tumor cell.

Non-limiting examples of suitable cancer antigens include the following: NY-ESO-1; alpha-fetoprotein; Desmoyokin/AHNAK^{S2580F}; (e.g., for Relapsed or Refractory Multiple Myeloma); Cancer/testis antigen 1; CD7 (e.g., for treating T-cell leukemia or lymphoma); c-MET; DR5; (e.g., for treating neuroblastoma); Epstein-Barr virus; (e.g., for treating B-cell malignancies); ERBB2^{H473Y} and ERBB2IP^{E805G}; gp100; Histone H3 trimethylation; MAGEA1; MAGE-A3/A6; MAGEA4/8; Melan-A/MART-1; minor H antigen (HA-1); (e.g., for treating solid tumors or colorectal cancer); PRAME; TPBG (trophoblast glycoprotein) or 5T4 (e.g., for treating solid tumors such as colorectal, ovarian and gastric cancer, and childhood acute lymphoblastic leukemia (ALL)); TGFβRII frameshift antigen; VEGFR-2; Wilms tumor 1 (WT-1); KRAS-G12V; KRAS-G12D; TP53 R175H; AFP; KK-LC-1; RAC1-P29S; PIK3CA; LAGE-1A; P53; COL6A3; HA-2; HERV-E; and BRAF.

In some embodiments, said TCR is capable of specific binding to an HLA class I-presented epitope. In some embodiments, said TCR is capable of specific binding to an HLA-A-presented epitope. In a particular embodiment, said TCR is capable of specific binding to an HLA-A2-presented epitope. In another embodiment, said TCR recognizes (specifically binds) a tumor antigen (also referred to herein as a tumor associated antigen or TAA). In various embodiments, the tumor antigen is selected from the group consisting of: NY-ESO-1, KRAS (e.g. mutations G12C, G12D, and G12V), p53 (e.g. mutated residues R175, G245, R248, R249, R273, and R282), PIK3CA, PTEN, ERBB2 (HER2), AFP, KK-LC-1, RAC1-P29S, LAGE-1A, COL6A3, HA-2, HERV-E, BRAF, gp100, alpha-fetoprotein, Desmoyokin/AHNAKS2580F, Cancer/testis antigen 1, ERBB2H473Y, ERBB2IPE805G, minor H antigen (HA-1), PRAME, TPBG, 5T4, MAGEA1, MAGE-A3/A6, MAGEA4/8, Melan-A/MART-1, NRAS and Wilms tumor 1 (WT-1). In some embodiments, the tumor antigen is selected from the group consisting of: NY-ESO-1, KRAS (e.g. mutations G12C, G12D, and G12V), p53 (e.g. mutated residues R175, G245, R248, R249, R273, and R282), PIK3CA, PTEN, ERBB2 (HER2), AFP, KK-LC-1, RAC1-P29S, LAGE-1A, COL6A3, HA-2, HERV-E, BRAF, gp100, alpha-fetoprotein, Desmoyokin/AHNAKS2580F, Cancer/testis antigen 1, ERBB2H473Y, ERBB2IPE805G, minor H antigen (HA-1), NKG2DL, PRAME, TPBG, 5T4, MAGEA1, MAGE-A3/A6, MAGEA4/8, Melan-A/MART-1, NRAS and Wilms tumor 1 (WT-1). In some embodiments, the tumor antigen is a cancer testis antigen, including, but not limited to, NY-ESO-1 (CTAG1B), MAGE family (e.g. MAGEA1, MAGE-A3/A6, MAGEA4/8, MAGEC1, MAGEC3), BAGE family (e.g. BAGE1, BAGE2) GAGE family (e.g. GAGE1, GAGE2, GAGE3), SSX family (e.g. SSX2, SSX4), STAGE family (e.g. CTAGE-1, CTAGE-5), PAGE family (e.g. PAGE1, PAGE2, PAGE3), PRAME, Cancer/testis antigen 1 and LAGE-1A. In another particular embodiment, said tumor antigen is a mutated KRAS antigen, including, but not limited to, G12C, G12D, and/or G12V mutations. In another particular embodiment, said tumor antigen is a mutated NRAS including, but not limited to, codons 12, 13, and 61 mutations. In another particular embodiment, said tumor antigen is a mutated p53 antigen, including, but not limited to R175, G245, R248, R249, R273, and/or R282 mutations. In a particular embodiment, the epitope is other than a viral epitope.

In another embodiment, said tumor antigen is selected from the group consisting of NY-ESO-1 and LAGE-1A In a particular embodiment, the antigen is NY-ESO-1. Exemplary and advantageous TCRs directed to human NY-ESO-1 are disclosed and exemplified below.

In certain embodiments, the exogenous antigen receptors target a patient-specific and/or shared tumor neoantigen. The term “neoantigen” used herein denotes peptides that are absent from normal human tissues and expressed on a tumor of interest. Tumor neoantigens are generally known to those skilled in the art. Examples of shared neoantigens include, but are not limited to, KRAS (e.g. mutations G12C, G12D, and G12V), p53 (e.g. mutated residues R175, G245, R248, R249, R273, and R282), PIK3CA, PTEN, and ERBB2. In another embodiment, the neoantigen is selected from the group consisting of KRAS (e.g. mutations G12C, G12D, and G12V), p53 (e.g. mutated residues R175, G245, R248, R249, R273, and R282), PIK3CA and PTEN. Alternatively or in addition, the engineered MAIT cells disclosed herein are used to target an MR-1 expressing tumor cell. Yet in other embodiments, cell compositions of the invention are not used in the treatment of MR-1 expressing tumors.

According to other embodiments, the use of TCRs directed to additional antigens is contemplated. It will be understood by those skilled in the art that recognition of such antigens is typically MHC-dependent, more typically HLA class-I dependent.

In certain other embodiments, a TCR may be directed to a microbial antigen, a pathogen, a viral antigen, a fungal antigen, or a bacterial antigen, wherein each possibility represents a separate embodiment of the invention. In other embodiments, the target for the exogenous TCR is a microbial antigen. Examples of microbial antigens include, but are not limited to, viral antigens, bacterial antigens, and fungal antigens. In certain embodiments, the engineered MAIT cells disclosed herein are used to treat a disease or infection caused by the microbe expressing the antigen. A skilled artisan would appreciate that microbes comprise bacteria, viruses, fungi, and parasites, wherein microorganisms that cause disease are called pathogens. In some embodiments, as used herein the term “microbial antigen” may encompass a pathogenic target, i.e., a pathogenic antigen.

In some embodiments, the exogenous TCRs target a bacterial antigen, which is used, in some embodiments, to treat a bacterial infection. In certain embodiments, the targeted bacterium can be selected from Nitrospira spp., Nitrosospira spp., Nitrobacter spp., Nitrosomonas spp., Clostridium spp., Bacillus spp., methanogenic archaea, coliforms, Salmonella spp., Bacteroides spp., Staphylococcus spp., Streptococcus spp., Neisseria spp., Haemophilus spp., Bordetella spp., Listeria spp., Mycobacterium spp., Shigella spp., Pseudomonas spp., Brucella spp., Treponema spp., Mycoplasma spp., Yersinia spp., Vibrionaceae spp., Chlamydia spp., Legionella spp., Escherichia spp., Acinetobacter spp., Burkholderia spp., Thiobacillus spp., Rickettsia spp., Sphinomonas spp., Francisella spp., Campylobacter spp., and Helicobacter spp.

In some embodiments, the exogenous TCRs target a fungal antigen, which is used, in some embodiments, to treat a fungal infection. In one embodiment, the fungal infection is a yeast infection.

In another embodiment (for example, when the use of placenta-derived MAIT such as IVB MAIT is contemplated), the antigen is a viral antigen. As used herein, “viral antigens” comprise antigens expressed by viral proteins, including scenarios in which the antigens are currently expressed by either a viral or a cancer cell (e.g., in the case of an oncogenic protein). Accordingly, in some embodiments, the engineered MAIT cells recognizing a viral antigen are used to treat a viral infection; or, in other embodiments, to treat a malignancy expressing a viral antigen. In yet other embodiments, the antigen is other than a viral antigen.

Non-limiting examples of viral antigens include the following: hexon or penton, for example, for treating an adenovirus; HPV E6; HPV E7; immediate early-1 (IE-1) or tegument phosphoprotein of 65 kilodalton (pp65), for example, for treating cytomegalovirus (CMV); EBV nuclear antigen 1 (EBNA1), BZLF1, or products of any of the EBV latent genes LMP1, LMP2, EBNA1, EBNA2, EBNA3A, EBNA3B, or EBNA3C, for example, for treating Epstein-Barr virus (EBV) or lymphoma; VP1 or large T, for example, for treating BK virus (BKV); U11, U14, or U90, for example, for treating human herpesvirus 6 (HHV-6); herpes simplex virus-1 (HSV-1) thymidine kinase (HSV-TK), for example, for treating HSV-1.

In various embodiments, the antigen is characterized by surface expression of less than 200, 100, 80, 70, 50, 40, 30, 20 or 15 molecules per cell. Each possibility represents a separate embodiment of the invention.

### Cancer Testis Antigens

According to some embodiments, the invention provides compositions and methods comprising a population of engineered MAIT cells expressing an exogenous TCR directed to a cancer testis antigen. Cancer testis antigens are proteins that are normally expressed only in human testis germ cells but are aberrantly expressed in various types of cancer cells. They are characterized by restricted expression in normal adult tissues, primarily in testicular germ cells, whereas expression in other cells and tissues is typically absent in healthy adults. These antigens may be expressed in a wide range of tumor types and may be used to induce tumor-specific immunogenicity in cancer patients.

For example, without limitation, TAA including HLA class I-restricted epitopes may be derived from cancer testis antigens including: NY-ESO-1 (CTAG1B), MAGE family (e.g. MAGEA1, MAGE-A3/A6, MAGEA4/8, MAGEC1, MAGEC3), BAGE family (e.g. BAGE1, BAGE2) GAGE family (e.g. GAGE1, GAGE2, GAGE3), SSX family (e.g. SSX2, SSX4), STAGE family (e.g. CTAGE-1, CTAGE-5), PAGE family (e.g. PAGE1, PAGE2, PAGE3), PRAME, Cancer/testis antigen 1 and LAGE-1A

According to particular embodiments, the cancer testis antigen is selected from the group consisting of NY-ESO-1 and LAGE-1A. In some embodiments, cell compositions of the invention comprise a population of engineered MAIT cells expressing an exogeneous TCR directed to NY-ESO-1.

The term “NY-ESO-1” or “New York esophageal squamous cell carcinoma 1” refers to the well-known cancer-testis antigen (CTA) also known as cancer/testis antigen 1B (CTAG1B). The human CTAG1B gene maps to the Xq28 region of the X chromosome, and is silenced in normal somatic cells except for male testis. However, NY-ESO-1 is aberrantly expressed in many types of cancer cells as a consequence of an epigenetic event that involves tightly controlled recruitment and sequential interaction of histone deacetylases, histone methyltransferase, DNA methyltransferases, and transcription factors. A human NY-ESO-1 sequence is set forth in accession no. NP_001318.1.

NY-ESO-1 expression has been reported in a wide range of tumor types, including neuroblastoma, myeloma, metastatic melanoma, synovial sarcoma, bladder cancer, esophageal cancer, hepatocellular cancer, head and neck cancer, non-small cell lung cancer, ovarian cancer, prostate cancer, and breast cancer.

Various naturally-occurring or engineered TCR recognizing NY-ESO-1 are known in the art, for example those described in Thomas et al. (Front Immunol. 2018, 9:947) and in U.S. Pat. No. 10,201,597, WO2020188348, US20200040358 and U.S. Pat. No. 8,088,379, incorporated herein by reference.

In some embodiments, the TCR recognizes an epitope comprising amino acids 157-167 of human NY-ESO-1. For example, Robbins et al. (J. Immunol. 2008, 180:6116-6131) discloses variants of the 1G4 TCR, that recognizes a peptide corresponding to amino acid residues 157-165 of NY-ESO-1 (SEQ ID NO: 9) in the context of the HLA-A*02 class I allele. WO 2024/023826, to some of the present inventors and co-workers, also discloses engineered TCRs directed to this epitope in the context of HLA-A*02. The contents of these publications are hereby incorporated by reference. In some embodiments, TCRs in accordance with the invention bind specifically to an HLA-A2-presented epitope comprising the amino acid sequence of SEQ ID NO: 9, corresponding to residues 157 to 165 of human NY-ESO-1.

NY-ESO-1 shares sequence homology with its paralog CTAG2 (NY-ESO-2 or LAGE-A1), and accordingly TCRs may be designed to target both tumor antigens. For example, the peptide epitope of SEQ ID NO: 9 is characteristic of both NY-ESO-1- and LAGE-A1-expressing tumors.

According to particular embodiments, TCRs of the invention are directed to a peptide epitope of SEQ ID NO: 9 in the context of HLA-A*0201 and/or HLA-A*0206. As used herein, the term “HLA-presented” peptide, epitope or antigen refers to a peptide capable of specifically binding an antigen-binding groove of an MHC or a particular allele thereof (for example an HLA-A2-presented epitope is specific to HLA-A2 alleles). Such an antigen is commonly referred to in the art as being “restricted” by such an MHC. The antigen generally has a characteristic dimension and/or chemical composition—for example, a characteristic amino acid length and set of anchor residues, respectively, in the case of a peptide antigen-enabling it to specifically bind the antigen-binding groove of a particular MHC haplotype so as to form an MHC/antigen complex therewith having an antigen presenting portion capable of specifically binding a variable region of a cognate TCR. For HLA-A2, for example, the anchoring positions are P2 and P9.

According to exemplary embodiments, TCRs in accordance with the invention comprise complementarity determining region (CDR) sequences as set forth in Table 1 below, in which CDR₁α, CDR₂α and CDR₃α correspond to the CDR1, CDR2 and CDR3 sequences of the alpha chain, and CDR1B, CDR2B and CDR3B correspond to the CDR1, CDR2 and CDR3 sequences of the beta chain, respectively.

TABLE 1
Exemplary CDR sequences of anti-NY-ESO-1 TCR

| | Amino acid | | | |
|---|---|---|---|---|
| | sequence/ | | Nucleic acid sequence/ | |
| CDR | SEQ ID NO. | | SEQ ID NO. | |
| CDR₁α | DSAIYN | 1 | GACTCTGCCATCTACAAC | 13 |
| CDR₂α | IQSSQRE | 2 | ATCCAGAGCTCGCAGAGGGAG | 14 |
| CDR₃α | AVRPLYGGSYIPT | 3 | GCCGTGCGGCCTCTGTACGGAGGCTCTTATATCCCAAC | 15 |
| | | | C | |
| CDR₁β | MNHEY | 4 | ATGAATCACGAGTAC | 16 |
| CDR₂β | SVGAGI | 5 | TCCGTGGGAGCAGGAATC | 17 |
| CDR₃β | ASSYVGNTGELE | 6 | GCCTCCTCTTATGTGGGCAACACCGGCGAGCTGTTC | 18 |

In a particular embodiment, said tumor antigen is NY-ESO-1. According to an exemplary embodiment, said TCR comprises a TCR α chain comprising a CDR1 having the amino acid sequence of SEQ ID NO: 1, a CDR2 having the amino acid sequence of SEQ ID NO: 2, and a CDR3 having the amino acid sequence of SEQ ID NO: 3, and a TCR β chain comprising a CDR1 having the amino acid sequence of SEQ ID NO: 4, a CDR2 having the amino acid sequence of SEQ ID NO: 5, and a CDR3 having the amino acid sequence of SEQ ID NO: 6. In a particular embodiment, said TCR comprises a TCR α chain having the amino acid sequence as set forth in SEQ ID NO: 7, optionally excluding the signal peptide at positions 1-20 thereof, and a TCR β chain having the amino acid sequence as set forth in SEQ ID NO: 8, optionally excluding the signal peptide at positions 1-21 thereof. In another embodiment, the TCR contains CDR sequences having certain modifications (typically conservative substitutions) as compared to CDR sequences described herein, such that the antigen specificity is retained. In some embodiments, analogs or derivatives as described herein contain no more than 3, no more than 2, or no more than 1 amino acid substitutions.

According to an advantageous embodiment, the TCR directed to NY-ESO-1 comprises:
(a) a TCR α chain comprising: i. a VR comprising a CDR1 having the amino acid sequence of SEQ ID NO: 1, a CDR2 having the amino acid sequence of SEQ ID NO: 2, and a CDR3 having the amino acid sequence of SEQ ID NO: 3; ii. a CR comprising a cysteine residue at position 47 thereof and the amino acid sequence as set forth in SEQ ID NO: 10 at positions 250-254 thereof;
(b) a TCR β chain comprising: i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and ii. a CR comprising a cysteine residue at position 57 thereof; and
(c) a plurality of interchain disulfide bonds between said α chain and β chain.

For example, the amino acid sequences of the full-length α and β chain precursors (including the signal peptides, which are underlined) in accordance with particularly advantageous embodiments of the invention are as set forth in SEQ ID NOs: 7-8, respectively, as follows:

(α chain precursor, SEQ ID NO: 7)
MFETLLGLLILWLQLQWVSSKQEVTQIPAALSVPEGENLVLNCSFTDSAIYNLQWFRQDPGKGL
TSLLLIQSSQREQTSGRLNASLDKSSGRSTLYIAASQPGDSATYLCAVRPLYGGSYIPTFGRGT
SLIVHPYIQNPDPAVYQLRDSKSSDKSVCLFTDFDSQTNVSQSKDSDVYITDKCVLDMRSMDFK
SNSAVAWSNKSDFACANAFNNSIIPEDTFFPSPESSCDVKLVEKSFETDINLNFQNLLVIVLRI
LLLKVAGENLLMTLRLWSS.
(β chain precursor, SEQ ID NO: 8)
MSIGLLCCAALSLLWAGPVNAGVTQTPKFQVLKTGQSMTLQCAQDMNHEYMSWYRQDPGMGLRL
IHYSVGAGITDQGEVPNGYNVSRSTTEDFPLRLLSAAPSQTSVYFCASSYVGNTGELFFGEGSR
LTVLEDLNKVFPPEVAVFEPSEAEISHTQKATLVCLATGFFPDHVELSWWVNGKEVHSGVCTDP
QPLKEQPALNDSRYCLSSRLRVSATFWQNPRNHFRCQVQFYGLSENDEWTQDRAKPVTQIVSAE
AWGRADCGFTSVSYQQGVLSATILYEILLGKATLYAVLVSALVLMAMVKRKDF.

In yet other embodiments, TCRs may include certain modifications (e.g. substitutions) to the amino acid sequences as set forth in SEQ ID NOs: 7 and 8 which retain a high degree of homology (e.g. greater than 95%, 96% 97%, 98% or 99%), as long as the structural elements as set forth at clauses (a) to (c) above are maintained. In a particular embodiment, the substitutions are conservative substitutions.

The terms “homology” and “sequence identity” as used herein refer to the degree of relatedness between two or more amino acid sequences, or two or more nucleic acid sequences, as determined by comparing the sequences. The comparison of sequences and determination of sequence identity or homology may be accomplished using a mathematical algorithm; those skilled in the art will be aware of computer programs available to align two sequences and determine the percent identity between them. The term “homology” refers in particular to the percentage of amino acid residues or nucleotides in a sequence that are identical with the residues of the reference polypeptide or polynucleotide with which it is compared, after aligning the sequences and in some embodiments after introducing gaps, if necessary, to achieve the maximum percentage homology, and not considering any conservative substitutions. As used here, the term “% identity,” which may be used interchangeably with the term “sequence identity”, in the context of two or more nucleic acid or polypeptide sequences, refer to two or more sequences that have a specified percentage of nucleotides or amino acid residues that are the same, when compared and aligned for maximum correspondence, as measured using a suitable sequence comparison algorithm (e.g., BLASTP and BLASTN or other algorithms available to persons of skill) or by visual inspection. Further, with respect to sequence identity as used herein, the overall length of the molecules compared is also taken into consideration, such that the degree of identity is calculated over the entire length of the sequences rather than locally.

### Genetic Engineering of MAIT Cell Populations

The terms “engineering” and “genetic engineering” as used herein in connection with a MAIT cell population may be used interchangeably and refer to the genetic modification of the population, whether transiently or permanently. Engineered MAIT cells preferably express a heterologous molecule such as a TCR transgene as disclosed herein. Engineered MAIT cells are therefore expressly distinct from naturally occurring cell populations such as primary MAIT cells and from non-genetically modified expanded MAIT cells. Such modifications are conveniently performed using available recombinant methods, which may further employ additional technologies such as viral and non-viral transduction, mRNA electroporation, LNP, transposons, gene editing (e.g. using CRISPR-Cas9 systems and the like).

Recombinant methods for designing, expressing and purifying proteins, peptides and nucleic acid molecules are known in the art (see, e.g. Sambrook et al., 2001, Molecular Cloning: A Laboratory Manual, Cold Springs Harbor Laboratory, New York). Nucleic acid molecules may include DNA, RNA, or derivatives of either DNA or RNA. An isolated nucleic acid sequence encoding a polypeptide or peptide can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, modified nucleic acid sequences in which nucleotides have been inserted, deleted, substituted, and/or inverted in such a manner that such modifications do not substantially interfere with the nucleic acid molecule's ability to encode a functional product. A polynucleotide or oligonucleotide sequence can be deduced from the genetic code of a protein, however, the degeneracy of the code must be taken into account, as well as the allowance of exceptions to classical base pairing in the third position of the codon, as given by the so-called “Wobble rules”. Polynucleotides that include more or less nucleotides can result in the same or equivalent proteins. Using recombinant production methods, selected host cells, e.g. of a microorganism such as E. coli or yeast, are transformed with a hybrid viral or plasmid DNA vector including a specific DNA sequence coding for the polypeptide and the polypeptide is synthesized in the host upon transcription and translation of the DNA sequence.

Such recombinant methods may also be used in the preparation of nucleic acid constructs, including in particular expression constructs or vectors used for delivering and expressing an exogeneous TCR in an appropriate host cell such as in MAIT cell populations, as detailed herein. The constructs comprise nucleic acid molecules of the invention, and may also comprise regulatory sequences or selectable markers, as known in the art. The nucleic acid construct (also referred to in some embodiments as a vector) may include additional sequences that render this vector suitable for replication and integration in prokaryotes, eukaryotes, or optionally both (e.g., shuttle vectors). In addition, a typical cloning vector may also contain transcription and translation initiation sequences, transcription and translation terminators, and a polyadenylation signal.

In another embodiment, the nucleic acid construct comprises at least one nucleic acid molecule encoding the TCR α and β chains as disclosed herein. In another embodiment, the at least one nucleic acid molecule is operatively linked to one or more transcription control elements.

The phrase “operably linked” refers to a nucleic acid sequence linked a to a transcription control sequence in a manner such that the molecule is able to be expressed when transfected (i.e., transformed, transduced, infected, or transfected) into a host cell. Transcription control sequences are sequences, which control the initiation, elongation, and termination of transcription. Particularly important transcription control sequences are those which control transcription initiation, such as promoter, enhancer, operator and repressor sequences.

For example, in an expression vector capable of expressing a nucleic acid molecule in human MAIT cells, expression control sequences operatively linked to the gene product to be expressed include promoters and other elements that are active in human MAIT cells. The promoter can be of genomic origin or synthetically generated. A variety of promoters for use in lymphocytes including MAIT cells are well-known in the art. The promoter can be constitutive or inducible, where induction is associated with the specific cell type or a specific level of activation or maturation, for example. According to an exemplary embodiment, the promoter is Human eukaryotic translation elongation factor 1 α1 promoter, EF1A. Alternatively, a number of well-known viral promoters are also suitable. Promoters of interest include but are not limited to the β-actin promoter, SV40 early and late promoters, immunoglobulin promoter, human cytomegalovirus promoter, retrovirus promoter, and the Friend spleen focus-forming virus promoter. The promoters may or may not be associated with enhancers, wherein the enhancers may be naturally associated with the particular promoter or associated with a different promoter.

In another embodiment, the invention utilizes an expression vector comprising at least one nucleic acid construct as disclosed herein. The term “expression vector” as used herein, refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include suitable vectors known in the art, such as cosmids, plasmids (e.g., naked or contained in liposomes) and viruses (e.g., lentiviruses, retroviruses, adenoviruses, and adeno-associated viruses) that incorporate the recombinant polynucleotide. In another embodiment, the vector is a viral vector. In a particular embodiment, said vector is a retroviral vector.

Suitable viral vectors are known in the art and available commercially. Exemplary retroviral vectors include, without limitation, pMSGV1, pMSCV, pBMN, pQXIX, pBullet, pBabe, pMSGV, pRETRO, pMIGR, pMX, and pRET. Exemplary lentiviral vectors include, without limitation, pRRL, pCLX, pLenti, pHR, pLVX, and pCAG. Additional exemplary vectors include without limitation, pBullet, pLSC, adenoviral vectors (including singly and multiply replication deficient forms thereof), including, but not limited to adenovirus type 5 (Ad5) and adenovirus type 35 (Ad35), and adeno-associated viral (AAV) vectors including, but not limited to pAAV and pX601. In another embodiment, said vector is a retroviral vector comprising LTRs from the murine stem cell virus (MSCV LTRs), e.g. a pMSGV1 or pMSCV-derived vectors.

### Pharmaceutical Compositions

In some embodiments, there is provided a pharmaceutical composition comprising a pharmaceutical acceptable carrier and the population of engineered MAIT cells disclosed herein.

In some embodiments, a composition comprises an engineered MAIT cell expressing an exogenous antigen receptor. As used herein, the terms “composition” and pharmaceutical composition” may in some embodiments be used interchangeably having all the same qualities and meanings. In some embodiments, disclosed herein is a pharmaceutical composition for the treatment of a condition or disease as described herein.

In some embodiments, disclosed herein is a pharmaceutical composition comprising a pharmaceutically acceptable carrier and a population of engineered MAIT cells described herein.

A skilled artisan would appreciate that a “pharmaceutical composition” may encompass a preparation of one or more of the active ingredients described herein with other chemical components such as physiologically suitable carriers and excipients. The purpose of a pharmaceutical composition is to facilitate administration of a compound to an organism.

A skilled artisan would appreciate that the phrases “physiologically acceptable carrier”, “pharmaceutically acceptable carrier”, “physiologically acceptable excipient”, and “pharmaceutically acceptable excipient”, may be used interchangeably may encompass a carrier, excipient, or a diluent that does not cause significant irritation to an organism and does not abrogate the biological activity and properties of the administered active ingredient.

Techniques for formulation and administration of drugs or pharmaceutical compositions are found in “Remington's Pharmaceutical Sciences,” Mack Publishing Co., Easton, PA, latest edition, which is incorporated herein by reference.

In some embodiments, the composition or pharmaceutical composition disclosed herein is an injectable composition that is manufactured by adding one or more excipients, e.g., stabilizers and aqueous buffers, to the population of engineered MAIT cells described herein.

Alternatively or in addition, the engineered MAIT cells described herein have been expanded at least 100 fold, in other embodiments at least 200 fold, in other embodiments at least 400 fold, in other embodiments at least 600 fold, in other embodiments at least 1000 fold, in other embodiments at least 1500 fold, in other embodiments at least 2000 fold, in other embodiments at least 3000 fold, and still in other embodiments at least 5000 fold compared to day 0 of expansion, before administration to a patient.

In some embodiments, the method of preparing the pharmaceutical composition comprising the engineered MAIT cells described herein includes steps for freezing, e.g., cryopreserving, the cells, either before or after isolation, incubation, and/or engineering. Examples of suitable freezing agents are generally known in the art.

In other embodiments, for injection, the engineered MAIT cells disclosed herein may be formulated in aqueous solutions, preferably in physiologically compatible buffers such as Hank's solution, Ringer's solution, or physiological salt buffer, optionally in combination with medium containing cryopreservation agents.

In other embodiments, there is provided a pharmaceutical composition, comprising the engineered MAIT cells disclosed herein, wherein the composition is indicated for treating or ameliorating any of the diseases, disorders, and complications mentioned herein, each of which represents a separate embodiment.

One may, in various embodiments, administer the pharmaceutical composition disclosed herein in a systemic manner as generally known in the art. Alternatively, one may administer the pharmaceutical composition locally, for example, via injection of the pharmaceutical composition directly into a tissue region of a patient, such as, in non-limiting embodiments, intratumoral or intralesional administration. In other embodiments, the engineered MAIT cells are administered intramuscularly, intravenously, subcutaneously, or intraperitoneally, each of which is considered a separate embodiment. In this regard, “intravenous” comprises administration into a vein of a subject. In some embodiments, the pharmaceutical composition is administered intralymphatically as previously described in the art. For example, administration routes particularly suitable for adoptive transfer include systemic (e.g. intravenous or intraperitoneal) or local injection (e.g. intratumorally or intrathecally, for example into the cerebrospinal fluid in brain tumor).

Effective dosages and schedules for administering the compositions may be determined empirically, and making such determinations is within the skill in the art. The dosage can be adjusted by the individual physician in the event of any counterindications. Exemplary effective amounts of engineered MAIT cells to be used in ACT compositions are provided hereinbelow.

Depending on the severity and responsiveness of the condition to be treated, dosing can be of a single or, in other embodiments, a plurality of administrations, until alleviation of the disease state is achieved.

In certain embodiments, the treatment methods described herein further includes lymphodepleting, or in other embodiments immunosuppressing, the recipient prior to treatment. In other embodiments, reversible lymphodepletion or immunosuppression extends the biological half-life of the transplanted cells. Methods for lymphodepletion and immunosuppression of patients are known in the art. In some embodiments, lymphodepletion or immunosuppression is not necessary for administration of the engineered MAIT cells disclosed herein, at least in part because of their low immunogenicity.

In various embodiments, engraftment of the engineered MAIT cells in the host is not required for the cells to exert the described therapeutic effects, each of which is considered a separate embodiment. In other embodiments, engraftment is required for the engineered MAIT cells to exert the effect(s).

In certain embodiments, the subject treated by the methods and compositions described herein has a tumor. In other embodiments, the subject has a bacterial infection. In some embodiments, the subject has a viral infection. In some embodiments, the subject has a fungal infection. In some embodiments, the subject is a human. In another embodiment the subject does not have an infection (e.g. viral infection).

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including lymphoid cells. In another aspect, the kits and articles of manufacture comprise a label, instructions, and packaging material, for example for treating a disorder or therapeutic indication mentioned herein.

### Adoptive Transfer Cell Therapy (ACT)

As used herein, and unless otherwise specified, the term “adoptive transfer” refers to a form of passive immunotherapy where previously sensitized immunologic agents (e.g., cells or serum) are transferred to the recipients. The phrases “adoptive transfer cell therapy”, “adoptive cell transfer”, “adoptive transfer immunotherapy”, “adoptive transfer therapy”, “adoptive cell therapy” and “adoptive cell immunotherapy” are used interchangeably herein to denote a therapeutic or prophylactic regimen or modality, in which effector immunocompetent cells are administered (adoptively transferred) to a subject in need thereof, to alleviate or ameliorate the development or symptoms of cancer or infectious diseases.

The adoptively transferred cells are often directed to a tumor or a tumor-associated antigen, for example NY-ESO-1. In particular, ACT compositions of the invention contain an effective amount of MAIT engineered to express a TCR as disclosed herein. Thus, an ACT composition in accordance of the invention (also referred to herein as a composition “adapted for ACT”) contains effective amounts of viable engineered cells as disclosed herein, which are produced under sterile and suitable (e.g. cGMP grade) conditions, to be administered to a human subject as described herein as part of their therapeutic (e.g. anti-tumor) regimen. Further, the administered cells in an ACT composition are typically non-attenuated, and are capable of proliferating in the recipient subject in the presence of the relevant antigen (e.g. tumor antigen).

As used herein in connection with cell compositions, an effective amount (also referred to herein as a therapeutically effective amount) is an amount sufficient to induce or enhance a beneficial immune response such as an anti-tumor response when administered to a subject, e.g. 10⁷ to 10¹² cells or 10⁸ to 10¹² cells. In particular, with respect to the ACT compositions of the invention, the effective amount is an amount sufficient to induce or enhance said immune response upon adoptive transfer to a subject in need thereof, and typically comprises at least 10⁹ and more typically at least 5×10⁹ cells, and up to about 10×10¹⁰ cells or more, e.g. 5×10⁹-10×10⁹ viable cells provided by a preparation process as disclosed herein.

In one aspect, there is provided a cell composition comprising a population of engineered mucosal-associated invariant T (MAIT) cells expressing an exogenous T cell receptor (TCR), wherein the MAIT cells are derived from placenta, optionally further comprising a pharmaceutically acceptable carrier.

In one embodiment, the MAIT cells are derived (obtained) from placental intervillous blood (IVB). In another embodiment, the composition is adapted for adoptive transfer cell therapy (ACT). In some embodiments, the composition comprises at least about 10⁷ or 10⁸ viable cells of said population of engineered MAIT cells, and typically 10⁹-10¹¹, 5×10⁹-10¹¹ or 10¹⁰-10¹¹ cells of said population.

According to the present disclosure, cell compositions of the invention are characterized by unique structural properties providing for advantageous functional properties, as will be further described and exemplified hereinbelow.

As disclosed herein, cell compositions in accordance with the invention contain a population of substantially purified engineered MAIT cells, such that the composition contains less than 10% and typically less than 8%, 7%, 5%, 3%, 2%, 1% or 0.5% of other cell types. Typically, the cell composition comprises a population of ex-vivo expanded MAIT cells, which have been engineered to express an exogeneous TCR as disclosed herein. In some embodiments, In some embodiments, cell compositions of the invention comprise at least 90% TCRVα7.2⁺CD161⁺ cells, e.g. at least 93%, 95%, 98%, 99% or more. Typically and advantageously, cell compositions of the invention comprise at least 90% TCRVα7.2⁺ CD161^{high} cells, e.g. at least 93%, 95%, 98%, 99% or more. According to particular embodiments, surface expression levels of CD161 is characterized by less than 10%, 7%, 5%, 3%, 2% or 1% reduction as compared to primary MAIT cells isolated from placenta IVB (conveniently measured by flow cytometry as mean fluorescence intensity).

Cell compositions of the invention are characterized by expression of the exogeneous TCR (eTCR, which is typically an a/B TCR) on the surface of at least 20% of the cells and typically at least 30%, 40% or 50% of the cells. According to additional advantageous embodiments as demonstrated herein, preparation processes of the invention provide for the production of cell compositions of the invention are characterized by high surface expression levels of the eTCR. For example, more than 50% and typically at least 55% 60%, 65%, 70%, 75% or 80% of said cells may be characterized by surface expression of said eTCR, e.g. 70-80%, 75-85%, 70-90% or 80%-95% eTCR⁺ cells. In another embodiment, at least 75% and more typically at least 80% or 85% of said cells are CD8⁺ cells (e.g. 85-95%, 80-98% or 90-100%).

In another embodiment, cell compositions of the invention are characterized by substantially enhanced levels of one or more markers as compared to a control cell composition (such as a corresponding ACT composition of expanded peripheral blood T cells). In some embodiments, the at least one marker (characterized by substantially enhanced levels in the compositions of the invention) is selected from the group consisting of: CD69, MDR1, siglec-7 and KLRG1, wherein each possibility represents a separate embodiment of the invention. In some embodiments, at least 5% and typically at least 7%, 10%, 15%, 20%, 30% 40%, 45% or 50% of the cells are MDR1⁺ (e.g. 5-50%, 5-20%, 6-25%, 7-40% or 10-60%, or, in other embodiments, 20-50% or more). Thus, as opposed to conventional blood-derived T cells that express very low levels of MDR1⁺ (typically about 1% on CD8⁺ T cells), cell compositions of the invention are characterized by substantially enhanced occurrence of MDR1+ cells.

In another embodiment, cell compositions of the invention are characterized by substantially reduced levels of one or more markers as compared to a control cell composition. In some embodiments, the at least one marker (characterized by substantially reduced levels in the compositions of the invention) is selected from the group consisting of: CD73, CD45RA, CCR7 and CXCR4, wherein each possibility represents a separate embodiment of the invention.

In another embodiment, cell compositions of the invention are characterized by substantially enhanced levels of one or more cytokines (e.g. IFNγ, TNFα and/or IL-2) as compared to a control cell composition. In another embodiment, cell compositions of the invention are characterized by substantially enhanced levels of a plurality of the cytokines (e.g. 2 or 3) as compared to a control cell composition. In another embodiment, cell compositions of the invention are characterized by substantially enhanced levels of one or more cytotoxic T cell effector molecules (e.g. perforin and granzyme B) as compared to a control cell composition. In another embodiment, cell compositions of the invention are characterized by substantially enhanced levels of perforin and granzyme B as compared to a control cell composition.

In another embodiment, cell compositions of the invention are characterized by substantially reduced allo-reactivity as compared to a control cell composition. Thus, without wishing to be bound by a specific theory or mechanism of action, cell compositions may be used to induce antigen-specific cytotoxicity of a target cell in vivo while minimizing or delaying the rejection of the adoptively transferred MAIT cells by immune cells of the recipient subject, thereby improving therapeutic efficacy and/or safety.

In another embodiment cell compositions of the invention are further characterized by structural and functional properties (such as surface expression of additional markers) as disclosed herein and exemplified in the Examples below. Each possibility represents a separate embodiment of the invention.

While MAIT cells of the invention may be engineered to express, in addition to the exogeneous TCR, additional molecules or elements such as cytokines, chemokines or their receptors, the co-expression of other antigen-specific receptors such as chimeric antigen receptors (CAR) is explicitly excluded. Typically, cell compositions of the invention do not substantially comprise cells engineered to express a CAR.

In another aspect, there is provided a composition adapted for ACT, the composition comprising a substantially purified population of ex vivo-expanded MAIT cells engineered to express an exogenous TCR (eTCR), the population comprising at least 10⁹ viable cells, of which at least 90% are TCR-Vα7.2⁺ CD161^{high}.

In some embodiments, cell compositions of the invention are characterized by significantly enhanced occurrence of MAIT cells exhibiting a memory phenotype (e.g. effector memory MAIT cells) and/or expressing the CD8αα homodimer as compared to a control cell composition. In various embodiments, the control cell composition is prepared using conventional peripheral blood T cells. In another embodiment, the control cell composition is prepared using cord blood leukocytes. In another embodiment, the control cell composition is prepared using a control cell population as disclosed herein. Each possibility represents a separate embodiment of the invention.

In another embodiment, the composition may further comprise an additional active ingredient, e.g. an anti-cancer drug or a cytokine. In a particular embodiment, the anti-cancer drug is a cancer immunotherapy, e.g. an immune checkpoint inhibitor.

In some embodiments, there is provided a cell composition adapted for ACT, comprising a therapeutically effective amount of MAIT cells engineered to express a TCR directed to NY-ESO-1, the TCR comprising:
(a) a TCR α chain comprising: i. a VR comprising a CDR1 having the amino acid sequence of SEQ ID NO: 1, a CDR2 having the amino acid sequence of SEQ ID NO: 2, and a CDR3 having the amino acid sequence of SEQ ID NO: 3; ii. a CR comprising a cysteine residue at position 47 thereof and the amino acid sequence as set forth in SEQ ID NO: 10 at positions 250-254 thereof;
(b) a TCR β chain comprising: i. a VR comprising a CDR1 having the amino acid sequence of SEQ ID NO: 4, a CDR2 having the amino acid sequence of SEQ ID NO: 5, and a CDR3 having the amino acid sequence of SEQ ID NO: 6; and ii. a CR comprising a cysteine residue at position 57 thereof; and
(c) a plurality of interchain disulfide bonds between said α chain and β chain.

In another embodiment, the cell composition is prepared by a process as disclosed herein. In another embodiment, there is provided a process for preparing a cell composition as disclosed herein.

ACT compositions of the invention may be administered to a subject in need thereof by e.g. intravenous, intraperitoneal, intratumoral and/or intrathecal routes. In various embodiments, single-dose or multiple-dose administration regimens may be employed. For example, a single-dose administration route includes administration of an ACT composition comprising a therapeutically effective amount of engineered MAIT cells as disclosed herein, e.g. 10⁹-10¹¹ cells. A single-dose administration route may include administration of said ACT composition once every 3 months, 4 months, 5 months, 6 months or more (e.g. until remission or another therapeutic end-point is achieved). In another embodiment a single-dose administration route may include a single administration of said ACT composition. In other examples, a multi-dose administration route may include more frequent administrations of ACT compositions comprising lower amounts of engineered MAIT cells, so as to achieve the therapeutic outcome. For example, a dose of about 10⁷-10⁸ engineered MAIT cells may be administered weekly or once every 2, 3, 4, 5, 6, 7 or 8 weeks.

In another embodiment the cell compositions of the invention are prepared by a process comprising:
a. obtaining a MAIT-cell containing cell population,
b. incubating the cells in the presence of a MAIT cell antigen and IL-15, so as to produce a population of activated MAIT cells,
c. engineering the resulting activated MAIT cells to express an exogeneous TCR,
d. expanding the resulting engineered MAIT cells to obtain a therapeutically effective amount of said cells, and
e. isolating the resulting engineered MAIT cells.

In one embodiment, the MAIT-cell containing cell population is obtained (derived) from placenta. In another embodiment, the MAIT-cell containing cell population is advantageously obtained from IVB. In another embodiment, the method may further comprise subjecting the cell population obtained in a. to cryopreservation, which may be thawed prior to the incubation step (step b).

In another embodiment, the MAIT cell antigen used in step b. is a riboflavin metabolite. In another embodiment, said MAIT cell antigen comprises 5-OP-RU. For example, without limitation, 10-200 ng/ml IL-15 and 100-1000 nM 5-OP-RU may be used. Typically, step b. is performed in the absence of additional antigens or activating agents directed (or specific) to non-MAIT TCRs, such as a/β TCR antigens. In another embodiment, step b. is performed in the absence of anti-CD3 and/or anti-CD28 antibodies. In another embodiment step b. is performed in the absence of additional antibodies. In another embodiment step b. is performed in the absence of additional cytokines (such as IL-2). In another embodiment step b. is performed in the absence of additional T cell activating agents (such as cytokines and antibodies). In another embodiment step b. is performed in the absence of feeder cells. In another embodiment step b. is performed in the sole presence of the MAIT cell antigen (e.g. 5-OP-RU) and IL-15. According to exemplary embodiments, step b. is performed for a duration of 1-5 days or 2-4 days, e.g. 2, 3 or 4 days.

In various embodiments engineering the activated MAIT cells to express an exogeneous TCR may be performed by suitable methods, including, but not limited to, viral and non-viral transduction, transfection, mRNA electroporation, LNP, transposons, gene editing (e.g. using CRISPR-Cas9 systems and the like. In another embodiment, engineering the activated MAIT cells to express an exogeneous TCR is performed by transduction (e.g. by incubating said cells with a viral vector comprising a nucleic acid construct encoding an eTCR as disclosed herein). In some embodiments, fibronectin fragments (e.g. Retronectin) or other agents promoting co-localization of a viral vector and the intended target cells may be used to improve transduction efficacy. By means of a non-limiting example, the viral vector (e.g. a retroviral vector as disclosed herein) may be applied to a surface (e.g. plate or well) coated with retronectin or another suitable agent (e.g. using centrifugation), so as to provide a vector-coated surface, and incubating said coated surface (e.g. using centrifugation) with MAIT cells produced as disclosed herein.

In another embodiment, step d. is performed so as to obtain expansion by a factor of at least 100-fold (as compared to the number of MAIT cells obtained in a). In another embodiment expansion is by a factor of at least 120, 150, 200, 300, 350, 400, 500, 600, 700, 800, 900, 1,000 or more. In various embodiments, step d. is performed so as to obtain 10⁹-10¹¹, 5×10⁹-10¹¹ or 10¹⁰-10¹¹ of said engineered MAIT cells. In another embodiment, step d. is performed in the presence of IL-15. In another embodiment, step d. is performed in the absence of additional antibodies. In another embodiment step d. is performed in the absence of additional cytokines (such as IL-2). In another embodiment step d. is performed in the absence of additional T cell activating agents (such as cytokines and antibodies). In another embodiment step d. is performed in the absence of feeder cells. typically and advantageously, step d. is performed in the absence of MAIT cell antigens. In another embodiment step d. is performed in the presence of IL-15 as a sole cytokine supportive of cell proliferation. In another embodiment step d. is performed in the presence of IL-15 as a sole T cell activator. In yet other embodiments, a second expansion step, in the presence of additional antigens or activating agents directed to a/B TCR antigens (e.g. CD3 and/or CD28-specific antibodies) may be added following the IL-15 mediated expansion (e.g. after 7-10 days).

Typically, the process of the invention further comprises positive selection of TCRVα7.2-expressing cells, so as to obtain a population of substantially purified engineered MAIT cells. According to an advantageous embodiment, step e. comprises subjecting the resulting cells to positive selection of TCRVα7.2-expressing cells, so as to obtain a population of substantially purified engineered MAIT cells. Alternatively, the positive selection of TCRVα7.2-expressing cells is performed following step b. and before step c. In another embodiment, step e. is performed within 7-14 days of initiating step b., more preferably within 8-12 or 9-11 days, e.g. within 10 days of initiating step b. In some embodiments, positive selection of TCRVα7.2-expressing cells may be performed by cell sorting. In various embodiments, positive selection of TCRVα7.2-expressing cells may be performed using binding agents (e.g. antibodies) specific to the TCRVα7.2 chain, and employing suitable methodologies such as magnetic bead separation and flow cytometry. For example, step e. may be performed by flow cytometry-based sorting (e.g. by staining the cells with an antibody specific to TCRVα7.2 and subjecting said cells to fluorescence-activated cell sorting (FACS)). In another embodiment, magnetic bead separation is performed (e.g. using reagents and kits available e.g. from Miltenyi Biotech). According to a non-limiting example, biotinylated antibodies (such as the biotinylated anti-TCR Vα7.2 antibody TCR Vα7.2 anti-human antibody, Biotin REAfinity), may be used in conjunction with suitable avidin-containing magnetic beads.

In another embodiment, the process comprises large scale expansion using bioreactors suitable for large-scale clinical grade processes. e According to a non-limiting example, a three-dimensional (3D) bioreactor as disclosed in U.S. Pat. No. 11,939,562, incorporated herein by reference. For instance, such bioreactors may be used during the expansion step. In some embodiments, expansion is performed using a packed bed reactor. For example, without limitation, packed bed MiniBio reactors containing Fibra-Cel® disks may conveniently be used. Advantageously, the disks may be coated with FBS or other suitable coatings to create an ECM-like environment, facilitating the attachment and growth of antigen presenting cells.

### Immunotherapy and Special Patient Populations

In some embodiments, the cell compositions of the invention are for use in therapy. In another embodiment, cell compositions of the invention are for use in immunotherapy. In another embodiment the cell compositions of the invention are for use in the treatment of a subject having a tumor or malignancy. In some embodiments, cell compositions of the invention are fore use in treating a subject afflicted with a condition associated with expression of an HLA class I-restricted antigen.

As used herein, a condition associated with expression of an HLA class I-restricted antigen indicates a disease or disorder characterized by the presence of target cells expressing the antigen in the context of HLA class I molecules. Typically, the presence of such target cells contributes to the etiology and/or pathology of the disease or disorder.

In another aspect there is provided a method of treating a subject having a tumor or malignancy, comprising administering to said subject a cell composition as defined herein.

In another embodiment, the tumor or malignancy is characterized by expression of an HLA class I-restricted epitope of a tumor antigen. In various embodiments, the tumor antigen is selected from the group consisting of: NY-ESO-1, KRAS, p53, PIK3CA, PTEN, ERBB2 (HER2), AFP, KK-LC-1, RAC1-P29S, LAGE-1A, COL6A3, HA-2, HERV-E, BRAF, gp100, alpha-fetoprotein, Desmoyokin/AHNAKS2580F, Cancer/testis antigen 1, ERBB2H473Y, ERBB2IPE805G, minor H antigen (HA-1), PRAME, TPBG, 5T4, MAGEA1, MAGE-A3/A6, MAGEA4/8, Melan-A/MART-1, NRAS and Wilms tumor 1 (WT-1). In another embodiment, the tumor is a solid tumor. In another embodiment, the tumor is selected from the group consisting of melanoma, breast cancer, colon cancer, kidney cancer, liver cancer, lung cancer, ovary cancer, pancreatic cancer, prostate cancer, uterine cancer, cervical cancer, bladder cancer, stomach cancer, head and neck cancer, brain cancer, skin cancer and sarcoma. In another embodiment, the tumor is selected from the group consisting of melanoma, breast cancer, colon cancer, kidney cancer, lung cancer, ovary cancer, pancreatic cancer, prostate cancer, uterine cancer, cervical cancer, bladder cancer, stomach cancer, head and neck cancer, brain cancer, skin cancer and sarcoma. In another embodiment (for example, when the tumor antigen is NY-ESO-1), said tumor may be selected from the group consisting of: melanoma, myeloma, sarcoma, and bladder, brain (e.g. glioblastoma), ovarian, lung, breast, synovial and prostate tumors. In yet other embodiments, the treatment of hematopoietic tumors such as acute myeloid leukemia (AML) is contemplated. In a particular embodiment, said tumor is melanoma. In another embodiment, said subject is afflicted with a NY-ESO-1 expressing tumor or malignancy. In another embodiment said subject is HLA-A2-positive. In another embodiment said subject is afflicted with a NY-ESO-1 expressing tumor or malignancy and is HLA-A2-positive. In another particular embodiment, the subject is not afflicted with a hepatoma or hepatocellular carcinoma. In another particular embodiment, the subject is not afflicted with a liver cancer.

As disclosed herein, cell compositions in accordance with the invention are particularly effective even in the treatment of tumors and malignancies characterized by low expression levels of tumor antigens.

As used herein, the term “antigen density” refers to level of expression of said antigen on the surface of a target cell. When referred to in the context of cell populations, the term indicates the average surface expression of said antigen of the population. The density or the amount of a target antigen may be measured by various methods, such as semi-quantitative FACS, immunohistochemistry, or other immunoassays employing labeled antibodies directed to said antigen.

For example, high antigen densities (e.g. 1,000 antigen molecules per cell or more) of a target antigen (e.g., a tumor antigen expressed by a tumor cell) are generally considered sufficient to activate (through binding) a sufficient number of immune cells expressing a receptor specific to said antigen (including both TCR and CAR-based therapies), so as to induce a clinically-significant immune response. Such immune response diminishes with decreased antigen densities (e.g. below 200, 150, 100, 80, 60, 50 antigen molecules per cell or less) and may result in impaired therapeutic response or even resistance to treatment.

The term “low antigen density” unless specified elsewhere in the instant disclosure, generally refers to surface expression of less than 50 antigen molecules per cell. It is understood, that an antigen needs not be expressed in low densities over the entire population of target cells in order to impair durable therapeutic response. For example, the presence of a tumor cell population characterized by low density of a tumor antigen may evade the immune response and ultimately lead to the development of a treatment-resistance tumor, even following an initial therapeutic response. For example, low-density expression of an HLA-A-restricted antigen in accordance with the invention include surface expression of less than 50 complexes of peptide epitopes derived from the antigen which are presented in the context of HLA-A.

Accordingly, the term “low-density antigen” and related embodiments such as a condition (e.g. tumor) associated with expression of a low-density antigen refer to a situation in which there exists a target cell population in the subject to be treated that is characterized by surface expression of less than 50 antigen molecules per cell. Examples of low-density antigens to be targeted in connection with embodiments of the invention include for example NY-ESO-1 and other cancer testis antigens disclosed herein.

As disclosed herein, cell compositions in accordance with the invention are particularly effective even in the treatment of tumors and malignancies characterized by low expression levels of tumor antigens. As exemplified herein, cell compositions of the invention exhibited high cytotoxicity against target cells characterized by low antigen densities (e.g. 10-50 antigen molecules per cell) as compared to target cells characterized by antigen densities higher than 100 antigen molecules per cell. Thus, cell compositions of the invention may advantageously be used effectively even in the treatment of subjects that are not otherwise amenable for treatment with immunotherapies such as CAR T cells and antibody-based therapies (e.g. antibody-drug conjugates, radionuclide therapy) due to insufficient surface expression of disease-associated antigens (e.g. tumor antigens). In some embodiments, the subject is afflicted with a treatment-resistant tumor in which one or more previous courses of treatment (such as immunotherapies as described above) have failed. In another embodiment said tumor is MDR1-positive.

For example, cell compositions of the invention are herein demonstrated to exhibit antigen-specific cell lysis of target cells (e.g. evident by cleaved caspase 3) characterized by average antigen densities of 10-50 antigen molecules per cell. In addition, it is surprisingly demonstrated herein that compositions of the invention were particularly effective against tumor cells characterized by very low antigen densities of about 10-30 antigen molecules per cell than they were against tumor cells characterized by antigen densities of about 30-50 antigen molecules per cell.

In some embodiments, the engineered MAIT cells are autologous to the subject (obtained from the subject to be treated, autologous cell compositions). In other embodiments, the engineered MAIT cells are allogeneic to the subject (obtained from a donor, allogeneic cell composition). In some embodiments, allogeneic cell compositions to be used in the methods of the invention may be administered to the subject in a modified therapeutic regimen, in which multiple administrations of cell compositions (prepared from the same donor or different donors) which may contain sub-therapeutic amounts of engineered cells are provided to the subject so as to collectively administer a therapeutic amount of said cells (e.g. 10⁹-10¹¹ cells in total). In some embodiments, the cells have been obtained from a single donor (e.g. from IVB of a healthy donor). In other embodiments, the cells have been obtained from a plurality of donors.

It is to be understood, that as long as the exogeneous TCR is HLA-matched with the subject to be treated, cells of the allogeneic donor need not be histocompatible with the subject. As disclosed herein, MAIT cells exhibit a significantly reduced potential for allo-rejection than conventional peripheral blood T cells expressing an endogenous a/B TCR. Accordingly, partly or fully HLA-mismatched donors may conveniently be used, thereby improving the availability of treatments to various patient populations. In another embodiment, the donor of the engineered MAIT cells of said allogeneic composition is histocompatible with said subject. In another embodiment the donor of the engineered MAIT cells of said allogeneic composition is partly histocompatible with said subject. In another embodiment the donor of the engineered MAIT cells of said allogeneic composition is not histocompatible with said subject.

In another embodiment, cell compositions of the invention are used to induce antigen-specific cytotoxicity of a target cell, wherein the exogeneous TCR recognizes a low-density antigen of the cell. In another embodiment cell compositions of the invention are used for the treatment of a subject afflicted with a condition associated with a low-density antigen, and wherein said TCR recognizes the low-density antigen. According to particular embodiments, the low-density antigen is a tumor antigen as disclosed herein. In a particular embodiment, the low-density antigen is characterized by surface expression of less than 50, 40, 30, 20 or 15 molecules per cell. In certain other particular embodiments, the low-density antigen is a tumor antigen characterized by surface expression levels as disclosed herein. In another embodiment cell compositions of the invention are for use in immunotherapy. In another embodiment cell compositions of the invention are for use in inducing antigen-specific cytotoxicity of a target cell while minimizing or delaying host-versus graft reaction. In other embodiments, cell compositions and methods of the invention provide for enhanced persistence of the administered cells in the recipient subject as compared to a control cell composition (e.g. prepared from peripheral blood T cells). In other embodiments, cell compositions and methods of the invention provide for prolonged therapeutic activity as compared to the control cell composition.

### Additional Methods of Use

Described herein are methods of use of MAIT and engineered MAIT cells described throughout. In some embodiments, the engineered MAIT cells used in the methods disclosed herein, comprise engineered placental MAIT cells. In some embodiments, the engineered MAIT cells used in the methods described herein, comprise engineered IVB MAIT cells. In some embodiments, the source of the engineered placenta-derived MAIT cells used in the methods disclosed herein is maternal. In some embodiments, the source of the engineered placenta-derived MAIT cells used in the methods disclosed herein is fetal. In some embodiments, the source of the engineered placenta-derived MAIT cells used in the methods disclosed herein is both maternal and fetal. In some embodiments, the source of the engineered placenta-derived MAIT cells used in the methods disclosed herein is placenta from the decidua basalis region. In some embodiments, the source of the engineered placenta-derived MAIT cells used in the methods disclosed herein is placenta from the decidua parietalis region. In some embodiments, the source of the engineered placenta-derived MAIT cells used in the methods disclosed herein is placental intervillous blood.

In some embodiments, the source of placental MAIT comprises a combination of placental sources described herein. In some embodiments, the source of the placental MAIT used in methods disclosed herein is not cord blood.

In some embodiments, the MAIT cells used in the methods disclosed herein, comprise placental MAIT cells. In some embodiments, the MAIT cells used in the methods described herein, comprise IVB MAIT cells. In some embodiments, the source of the placenta-derived MAIT cells used in the methods disclosed herein is maternal. In some embodiments, the source of the placenta-derived MAIT cells used in the methods disclosed herein is fetal. In some embodiments, the source of the placenta-derived MAIT cells used in the methods disclosed herein is both maternal and fetal. In some embodiments, the source of the placenta-derived MAIT cells used in the methods disclosed herein is placenta from the decidua basalis region. In some embodiments, the source of the placenta-derived MAIT cells used in the methods disclosed herein is placenta from the decidua parietalis region. In some embodiments, the source of the placenta-derived MAIT cells used in the methods disclosed herein is placental intervillous blood. In some embodiments, the source of placental MAIT comprises a combination of placental sources described herein. In some embodiments, the source of the placental MAIT used in methods disclosed herein is not cord blood.

In some embodiments, the MAIT cells used in the methods disclosed herein are derived from a fetal blood source, a maternal blood source, a fetal and maternal blood source, a placental source, or a IVB source, or a combination thereof, wherein the MAIT cells may be engineered to express an exogenous antigen receptor or may not be engineered to express an exogenous antigen receptor. In some embodiments, MAIT cells are derived from a fetal blood source, a maternal blood source, a fetal and maternal blood source, a placental source, or a IVB source, or a combination thereof; and are not derived from cord blood wherein the MAIT cells may be engineered to express an exogenous antigen receptor or may not be engineered to express an exogenous antigen receptor.

In some embodiments of the methods of use described herein, the engineered MAIT cell comprises a TCR-MAIT cell, wherein the MAIT cell is derived from IVB. In some embodiments of the therapeutic methods described herein, a method comprises administering a population of TCR-MAIT cells to a subject in need.

In some embodiments, there is a provided a method of treating a subject having a tumor or malignancy, comprising the step of administering to the subject a population of engineered MAIT cells described herein. In some embodiments, there is provided a method of treating a subject having a tumor or malignancy, comprising administering to the subject a composition comprising the population of engineered MAIT cells disclosed herein. In some embodiments, the engineered MAIT cells are allogeneic to the subject.

In other embodiments, there is a provided a composition or a pharmaceutical composition comprising a population of engineered MAIT cells described herein for treating a subject having a tumor or malignancy. Those skilled in the art will appreciate, in light of the present disclosure, that tumors, malignancies, and hyperproliferative disorders can be treated with a population of engineered MAIT cells disclosed herein, particularly in cases where cells of the tumor, malignancy, or hyperproliferative disorders comprise (e.g., express, or in other embodiments contain) the recognized antigen. In some embodiments of methods of treating a subject having a tumor or malignancy, the engineered MAIT cells are derived from placental intervillous blood. In some embodiments, when treating a subject having a tumor or malignancy, the engineered MAIT cells are allogeneic to the subject. In some embodiments, when treating a subject having a tumor or malignancy, the engineered MAIT cells comprise an engineered TCR directed to a cancer antigen or tumor associated antigen, for example but not limited to those disclosed herein.

In some embodiments, there is provided a method of treating a subject infected with a pathogen, comprising the step of administering to the subject a population of engineered MAIT cells described herein. In other embodiments, there is provided a composition or a pharmaceutical composition comprising a population of engineered MAIT cells described herein for treating a subject infected with a pathogen. The pathogen can be a bacterial pathogen, a viral pathogen, or a fungal pathogen generally known in the art. Those skilled in the art will appreciate, in light of the present disclosure, that various infections can be treated with the engineered MAIT cells disclosed herein, particularly in cases where the pathogenic cells comprise (e.g., express, or in other embodiments contain) the recognized antigen. In some embodiments of methods of treating a subject infected with a pathogen, the engineered MAIT cells are derived from placental intervillous blood. In some embodiments, when treating a subject infected with a pathogen with engineered MAIT cells described herein, the engineered MAIT cells are allogeneic to the subject.

In some embodiments, method of treating a subject in need comprising use of any of the engineered MAIT cells described herein, comprise treating a subject having a tumor or malignancy, a subject infected with a pathogen.

In some embodiments, the exogenous TCR of the engineered MAIT cells comprises an antigen binding domain, wherein said antigen comprises NY-ESO-1, KRAS (mutations G12C, G12D, and G12V), P53 (residues R175, G245, R248, R249, R273, and R282), PIK3CA, PTEN, ERBB2, KRAS-G12V, KRAS-G12D, TP53 R175H, AFP, KK-LC-1, RAC1-P29S, PIK3CA, LAGE-1A, P53, COL6A3, HA-2, HERV-E, BRAF, alpha-fetoprotein; Desmoyokin/AHNAKS2580F; Cancer/testis antigen 1; Epstein-Barr virus, ERBB2H473Y and ERBB2IPE805G; HBV surface antigen; minor H antigen (HA-1); PRAME, TPBG, 5T4, Wilms tumor 1 (WT-1), gp100, MAGEA1; MAGE-A3/A6; MAGEA4/8; or Melan-A/MART-1.

The following examples are presented in order to more fully illustrate some embodiments of the invention. They should, in no way be construed, however, as limiting the broad scope of the invention.

### EXAMPLES

### Materials and Methods

Blood mononuclear cells were isolated from human placental intervillous blood (IVB) and separated by filtration and density gradient medium Lymphoprep™ (Ficoll). Red blood cells were depleted by RBC X1 lysis buffer. The isolated populations were cryopreserved in HI-FBS and dimethyl sulfoxide (DMSO) freezing solution. To analyze MAIT proportion and phenotype, cells were thawed, stained for MAIT markers as well as phenotypic markers (CD62L, CD45RA) and analyzed by flow cytometry. In some samples, MAIT % was analyzed before and after cryopreservation (no difference was observed). To compare MAIT % in IVB cells peripheral blood mononuclear cells (PBMC), PBMC were isolated from peripheral blood of unrelated healthy donors and analyzed similarly.

To generate MAIT cells, IVB mononuclear cells were thawed and MAIT cells activation was induced by 5-OP-RU (250 ng/ml) and IL-15 (50 ng/ml). On day 3 and 5 post-activation, cells were counted and analyzed by flow cytometry for MAIT %, and fresh medium and IL-15 was added. On day 7, cells were separated with magnetic beads and anti-TCR Vα7.2 antibody to further enrich for MAIT population, and the cells were cultured for another 3 days in the presence of IL-15. In some embodiments, the culture medium and culture conditions include CTS™ OpTmizer™ T cell expansion serum-free medium supplemented with 2.5% CTS™ immune cell serum replacement (Gibco™), at 37° C. with 5% CO₂.

For cell surface staining by flow cytometry, cells were placed in a 96-well microplate and washed with a staining buffer containing FBS and EDTA. Cells were then stained with a cocktail of fluorescently labeled antibodies for CD3, TCR Vα7.2 and CD161 to detect MAIT cells and additional antibodies as indicated. Cells were incubated for 20 min at 4° C., washed and resuspended in the staining buffer containing 7-AAD viability dye. Stained cells were analyzed on CytoFlex flow cytometer; results were then analyzed on FlowJo or Kaluza software. For intracellular staining by flow cytometry, cells were placed in a 96-well microplate in T cell medium supplemented with Phorbol 12-myristate 13-acetate (PMA) and ionomycin to induce T cell activation, and with brefeldin A and monensin (BD) to inhibit protein secretion. Cells were incubated at 37° C. for 4 hrs, then washed with staining buffer and stained with fixable viability dye followed by staining with cell surface antibodies as above. Cells then were fixed and permeabilized with the Cytofix/Cytoperm solution (BD) for 30 min at room temperature, washed with the Perm/Wash buffer (BD) and stained for 30 min at 4° C. in the Perm/Wash buffer containing fluorescently labeled antibodies for intracellular proteins (IFNγ, TNFα, perforin and granzyme B). Cells were washed and analyzed as above.

### Example 1. MAIT Cells are Generally More Abundant Among Placental IVB Mononuclear Cells than Among Peripheral Blood Mononuclear Cells (PBMC)

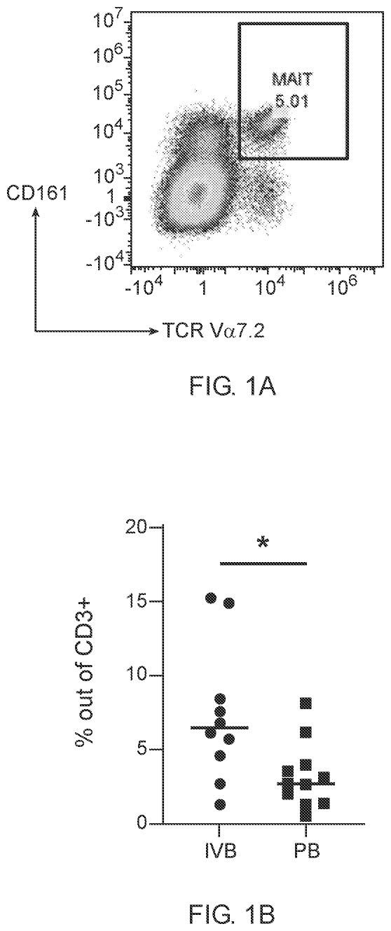

IVB samples and blood samples were obtained from healthy donors and separated by Ficoll gradient to isolate mononuclear cells as described above. The resulting cells were stained with antibodies as described above and analyzed by flow cytometry. The percentage of MAIT cells, as defined by expression of TCR Vα7.2 and CD161, was compared among placenta IVB and PBMC CD3⁺ cells from 13 and 11 (respectively) unrelated donors. As shown in FIGS. 1A-1B, although the proportion of MAIT cells (% out of CD3⁺) varied between donors in both peripheral blood and placenta, on average, there were significantly more MAIT cells among the CD3⁺ cells in IVB as compared to peripheral blood.

### Example 2. Phenotypic Differences Between Placental MAIT Cells, Peripheral MAIT Cells and Peripheral T Cells

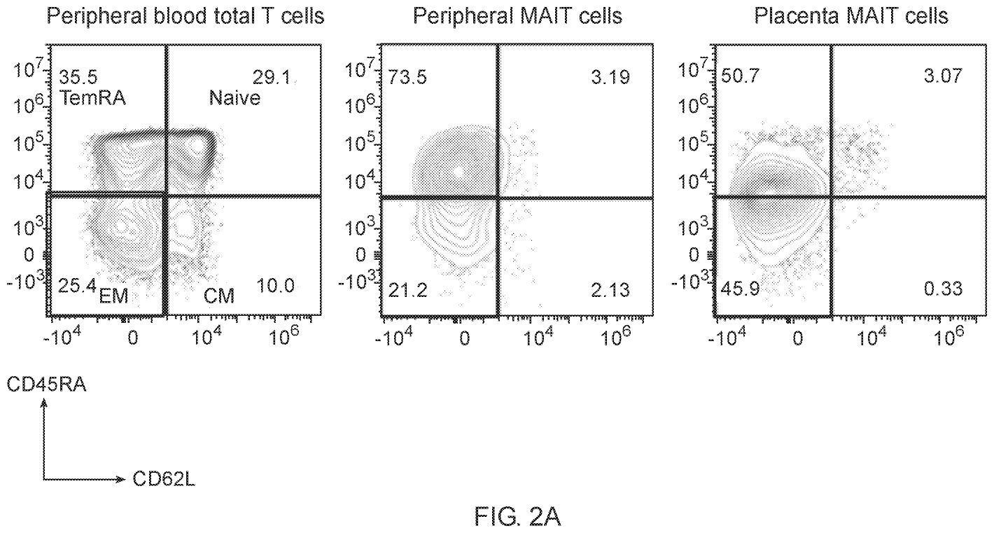

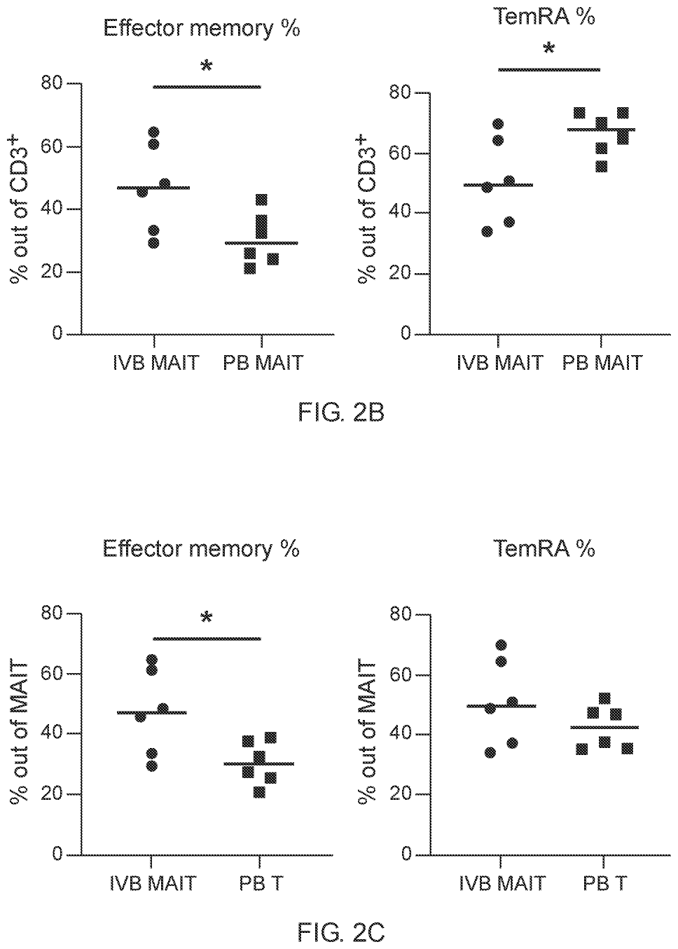

The phenotypes of T cells and MAIT cells from placenta IVB and peripheral blood (PB) were analyzed using CD62L- and CD45RA-specific antibodies. As can be seen in FIGS. 2A-2C, MAIT cells expressed low levels of CD62L and therefore were mostly either effector memory or terminally differentiated effector (TemRA) cells, unlike regular T cells, which also contained substantial naïve and central memory populations. Notably, MAIT cells derived from IVB had a significantly higher proportion of effector memory cells than MAIT cells derived from peripheral blood. Accordingly, they had less terminally differentiated effector cells than peripheral blood derived-MAIT cells (FIGS. 2A-2B). The results also indicate that IVB MAIT cells have lower CD45RA (p=0.035) than PB MAIT cells, and therefore the phenotype of IVB MAIT cells is less TemRA and more effector-memory. Effector memory cells might be a preferable phenotype for engineered T cells including MAIT cells, since they are more long-lived than terminal effectors, while they have better effector function and capabilities of migrating to peripheral tissues as compared to naïve and central memory cells. In addition, as can be seen in FIG. 2C, placental (IVB) MAIT cells contain significantly larger effector memory populations in comparison to conventional peripheral blood T cells (PB T), whereas the average difference in the abundance of terminal effectors did not reach statistical significance.

### Example 3. Expression of Chemokine Receptors is Higher on MAIT Cells from IVB Vs MAIT from Peripheral Blood

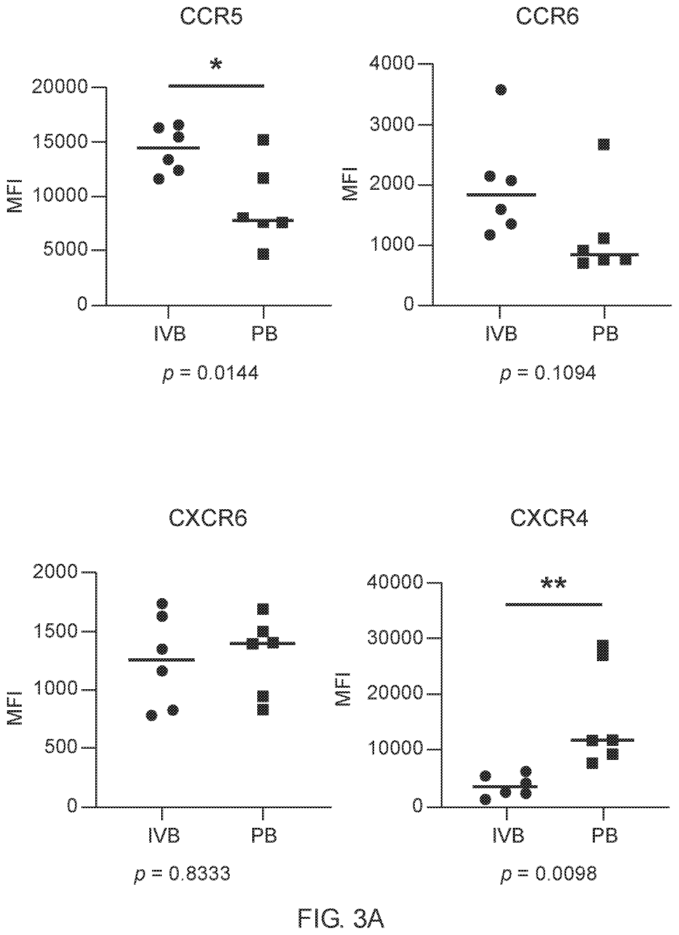

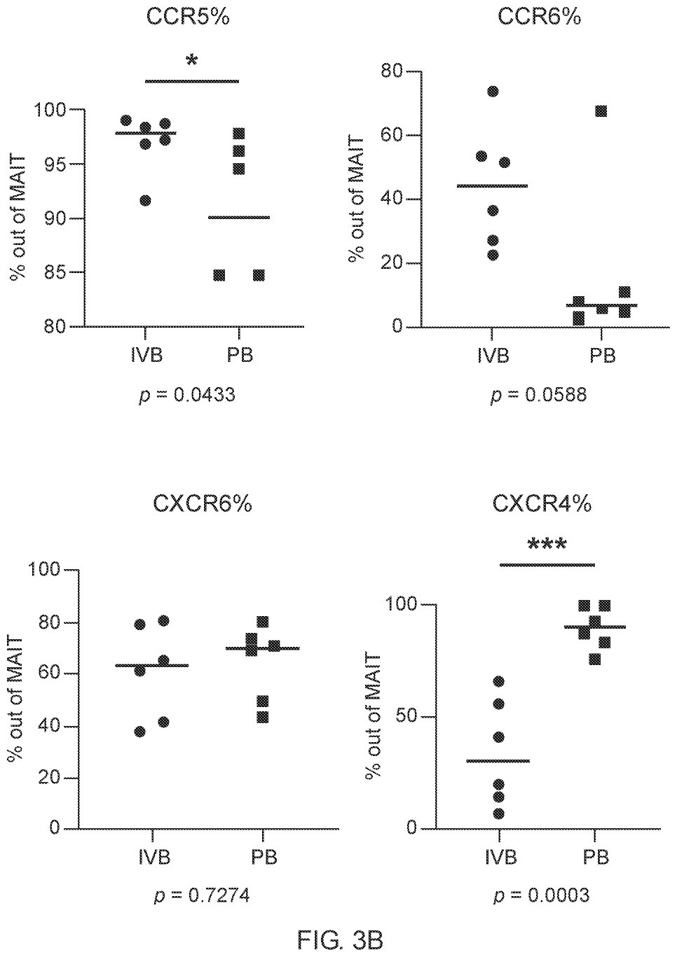

Chemokine receptors mediate migration of engineered T cells to peripheral tissues and to secondary lymphoid organs; they can also be important for infiltration of engineered-T cells into the tumor. The expression of chemokine receptors on MAIT cells from IVB and MAIT from peripheral blood (primary MAIT cells as described in Examples 1-2) was analyzed. It was found that placenta MAIT cells expressed significantly higher levels of CCR5 and lower levels of CXCR4, while CCR6 was also higher on IVB MAIT cells, albeit the difference did not reach statistical significance. CXCR6 expression was similar on MAIT cells from both sources (FIGS. 3A-3B). Increased CCR5 on IVB MAIT cells is expected to improve their trafficking to solid tumors. Decreased CXCR4 may have the same effect, because CXCR4 directs T cells to the bone marrow. In some solid tumors, the CXCR4 ligand SDF1 is highly expressed in the stroma, sequestering the T cells and preventing their infiltration into the core of the tumor. Accordingly, pharmacological inhibition of CXCR4 has been shown to improve the infiltration of cytotoxic T cells into the tumor and synergize with PD1 blockade in animal and in vitro models of solid tumors.

### Example 4. Functional Comparison Between MAIT Cells from IVB, Peripheral Blood MAIT Cells and Peripheral Blood T Cells

Perforin and granzyme B are molecules essential for cytotoxic function of effector lymphocytes such as T cells including MAIT cells. Perforin creates holes in the target cells, while granzyme B enters the target cells through these holes and induces apoptosis.

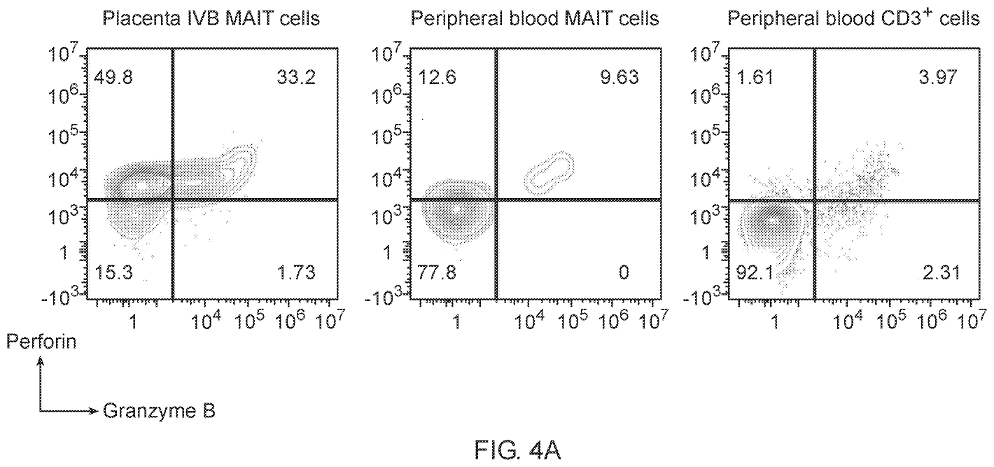

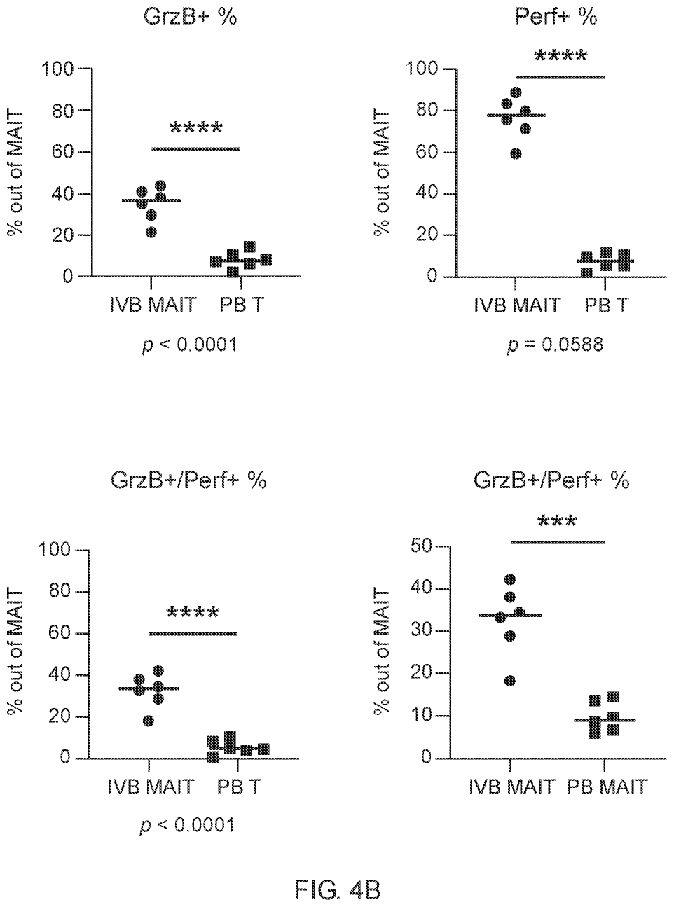

To evaluate perforin and granzyme B expression in the different cell populations, cells were isolated as described in Example 1 above were stimulated with PMA and ionomycin for 4 hours in the presence of protein secretion inhibitors. Cells were then were permeabilized, stained for MAIT markers or CD3 and the test molecules, and analyzed by flow cytometry. The results are presented in FIGS. 4A-4B.

In a comparative analysis, it was found that compared with peripheral blood MAIT cells, IVB MAIT cells express significantly higher levels of both effector molecules upon stimulation with PMA and ionomycin (FIGS. 4A-4B). In addition, the results show that MAIT cells contained significantly more cells producing perforin, granzyme B or both, in comparison to conventional T cells (PB CD3⁺).

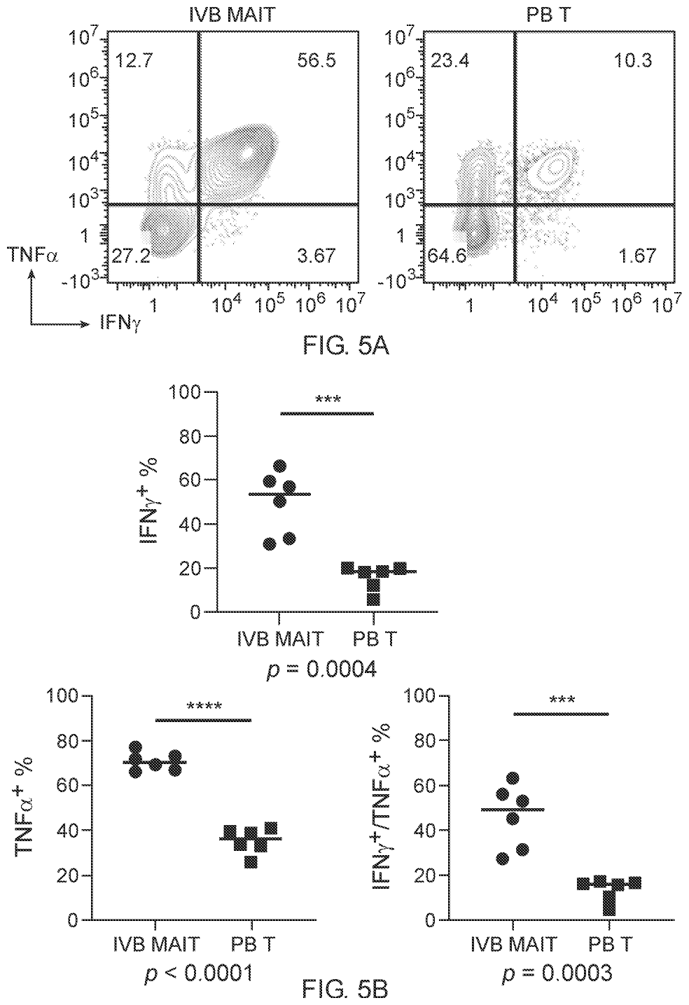

Cells were also analyzed similarly for cytokine expression (IFNγ, TNFα, IL2), and the results are presented in FIGS. 5A-5B. As can be seen, there were significantly more cells among the MAIT population than among the conventional T cells population, which produced IFNγ (IFNγ⁺), TNFα (TNFα⁺) or both (IFNγ⁺/TNFα⁺). In addition, MAIT cells from both sources (IVB and blood) were found to produce cytokines at similar levels, suggesting high cytotoxic potential of MAIT cells without triggering/inducing cytokine release syndrome (CRS) upon patient's treatment with engineered MAIT cells.

Thus, the results indicate that MAIT cells are more cytotoxic and function as stronger effectors than conventional peripheral blood T cells.

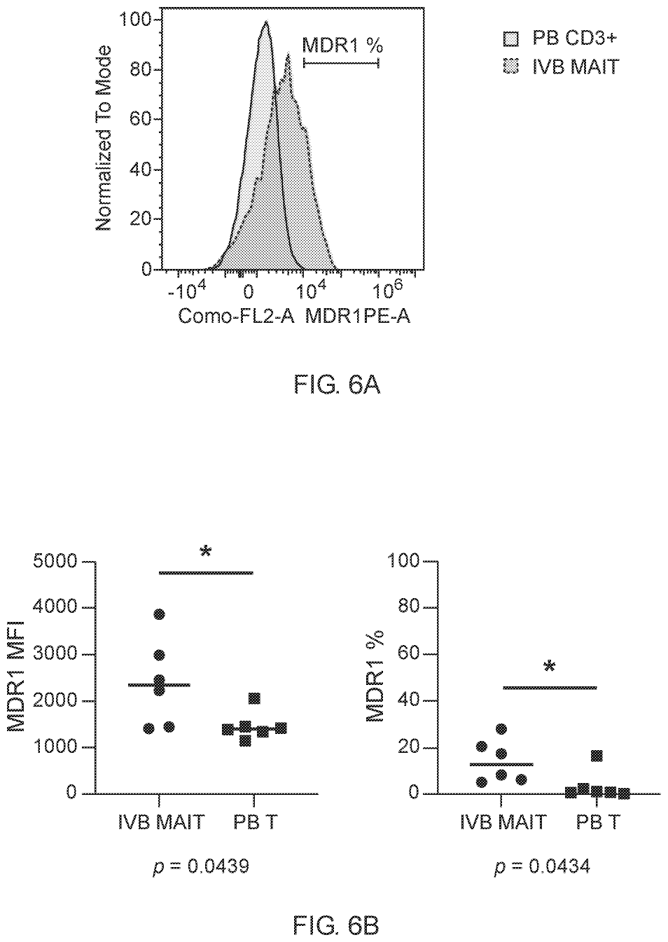

Finally, the expression of Multidrug Resistance 1 (MDR1) was similarly examined by flow cytometry. The results are presented in FIGS. 6A-6B, wherein FIG. 6A shows a representative sample of placenta IVB cells (gated on MAIT population) and peripheral blood T cells, and FIG. 6B is a quantitative phenotypical analysis of MDR1 protein expression on placenta MAIT cells and peripheral T cells (6 donors of each).

As can be seen, placental MAIT cells express significantly higher levels of MDR1. Accordingly, MAIT cells may be more resistant to chemotherapy and other drugs used in the treatment of patients in need of adoptive transfer therapy.

### Example 5. Phenotypic Characterization of Expanded MAIT Cells

Enrichment for MAIT Cells Via Activation of Placenta IVB Cells with 5-OP-RU

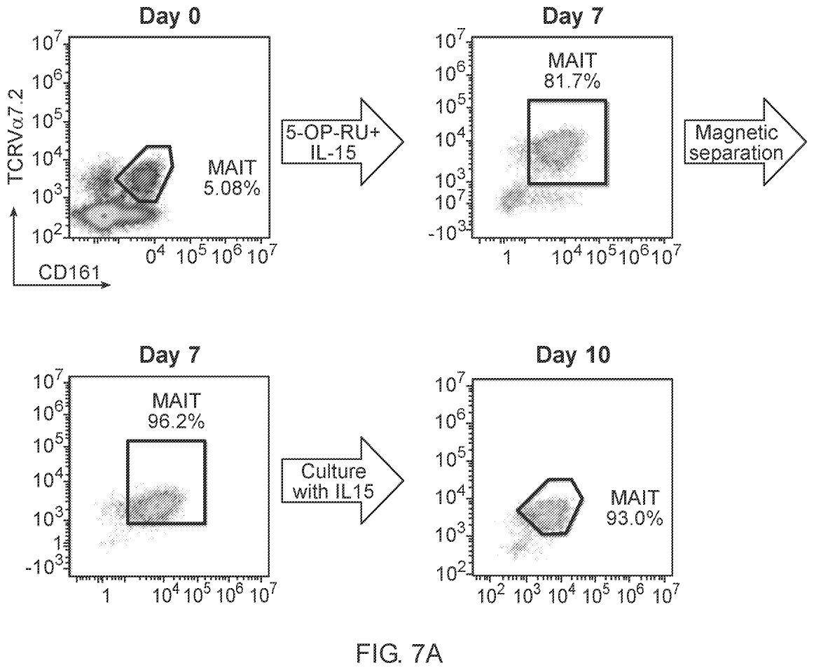

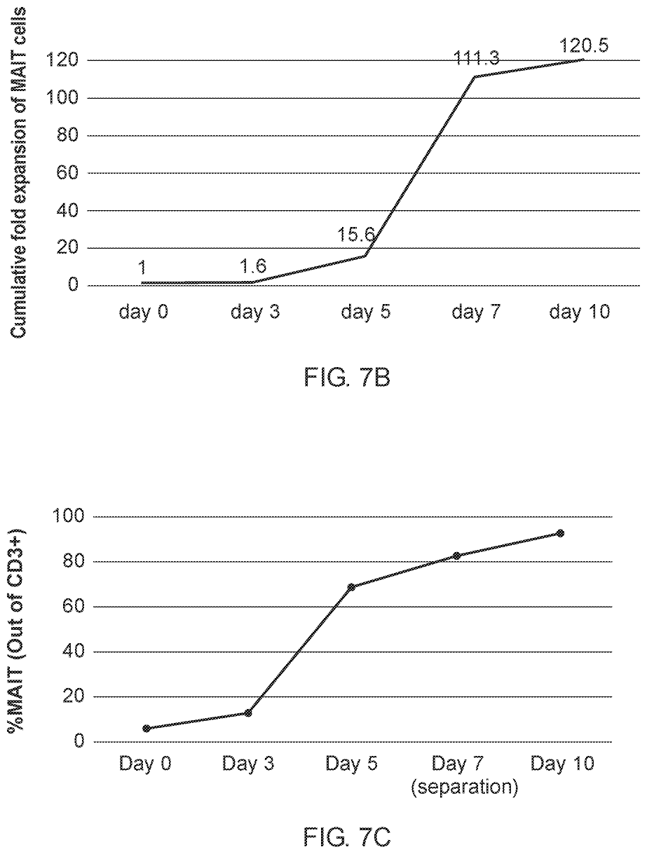

To obtain a population enriched with MAIT cells, IVB-derived mononuclear cells were activated with the MAIT cognate antigen, 5-OP-RU, and cultured in the presence of IL-15. After 7 days in culture, MAIT cells represented between 78% to 93% of cells in the CD3⁺ population, depending on the donor, and total fold expansion of MAIT cells was 46-112-fold (FIG. 7A). When MAIT % was lower than 90%, the cells were further enriched using anti-TCR Vα7.2 antibody and magnetic beads. Cells were cultured until day 10. The results are presented in FIGS. 7B-7C.

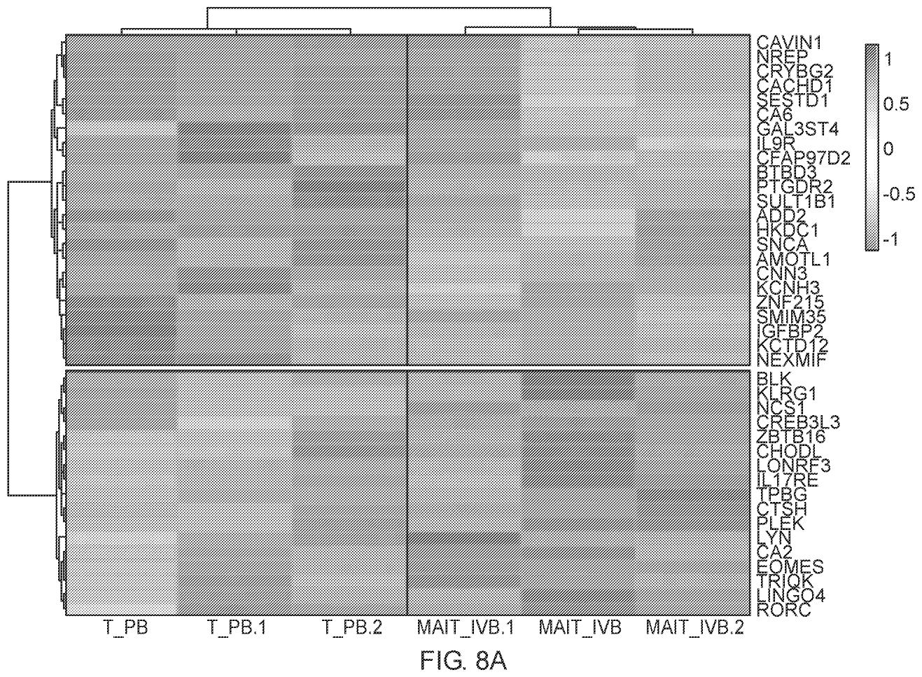

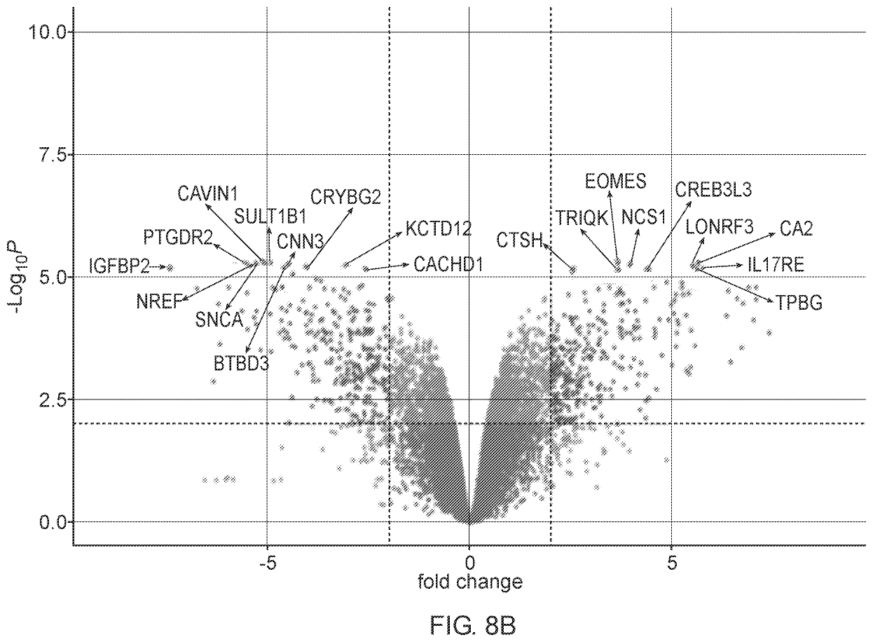

Since production of T cell compositions ultimately entails expansion of activated T cells, expanded placenta MAIT cells were compared to expanded conventional T cells. To this end, placenta IVB mononuclear cells were activated with 5-OP-RU and cultured in the presence of IL15, to enrich for MAIT population. On day 7 post-activation, MAIT cells were isolated using anti-TCR Vα7.2 antibody and magnetic beads. In parallel, PBMC were activated by anti-CD3 and anti-CD28 immobilized antibodies (TransAct™) and cultured with IL2; on day 7 post-activation, CD8⁺ T cells were isolated using anti-CD8 antibody and magnetic beads. MAIT cells were compared to CD8⁺ T cells to exclude differences between CD4+ and CD8⁺ T cells, since MAIT are mainly CD8⁺ and have very low population of CD4+. Three donors of each cell type were used. RNA was isolated from each sample and subjected to sequencing (RNAseq). The results are presented in FIGS. 8A-8B.

As can be seen from the results, multiple genes consistently differentiate between MAIT cells and regular CD8⁺ peripheral blood T cells, with some being higher in MAIT while other higher in regular T cells.

To analyze gene expression at the protein level, expanded MAIT cells and conventional T cells were assayed for expression of ˜360 proteins present on the LegendScreen™ panel (BioLegend). Cells were activated and expanded for 10 days and then MAIT and CD8⁺ T cells were isolated using magnetic beads. Purified cells of each cell type were stained with an anti-CD45 antibody with a different fluorophore, washed and combined, and then analyzed using the PE-conjugated panel of antibodies (LegendScreen™), per manufacturer's instructions. Experiment was repeated with another pair of donors. MAIT and T cells were gated on based on the CD45 fluorophore. Average median fluorescence intensity (MFI) for each marker was calculated between two donors; each marker was plotted with its average MFI in MAIT cells (X axis) and in peripheral CD8⁺ T cells (Y axis).

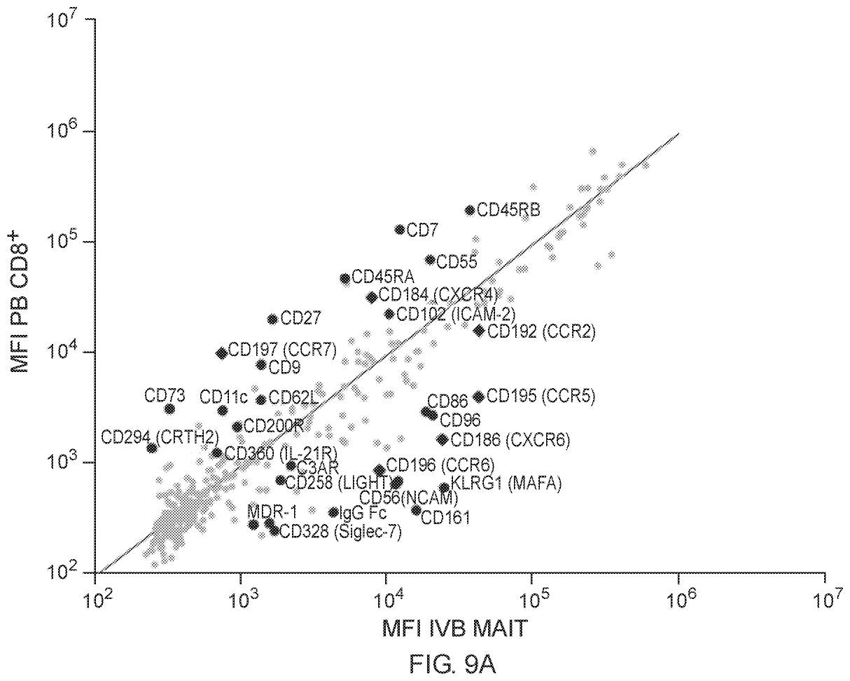

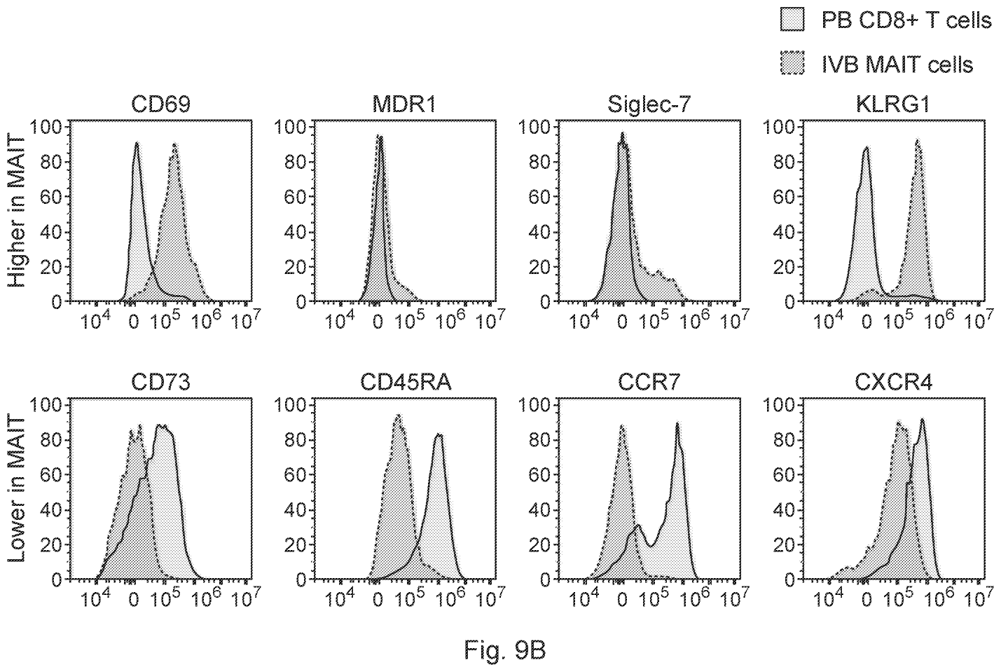

The results are presented in FIGS. 9A-9B. As can be seen, though most markers did not differ in intensity or % of positive cells between two cell types, some markers were either higher or lower in MAIT cells as compared to CD8⁺ T cells (shown in black). Notably, many chemokine receptors were differentially expressed on expanded MAIT cells (shown in black diamonds), with CCR2, CCR5, CCR6 and CXCR6 higher in MAIT cells, while CCR7 and CXCR4 higher in regular CD8⁺ T cells. This suggests that MAIT cells have a better potential to traffic to tissues, including solid tumors, while regular T cells are better equipped to traffic to lymph nodes and bone marrow.

As can be seen in FIG. 9B, expression of the following exemplary markers was significantly higher in MAIT cells as compared to conventional T cells: CD69, MDR1, siglec-7 and KLRG1. In addition, expression of the following exemplary markers was significantly lower in MAIT cells as compared to conventional T cells: CD73, CD45RA, CCR7 and CXCR4. Thus, MAIT cells retain significantly differential expression of various markers even following ex-vivo expansion.

To evaluate the results of the LegendScreen™ analysis in additional donors, expanded MAIT and T cells were stained for chemokine receptors and analyzed by flow cytometry. In this experiment, placenta IVB cells were activated on day 0 as described above; on day 5 cells were separated with anti-TCR Vα7.2 antibody to enrich for MAIT population, then pure MAIT cells were cultured with IL15 till day 12. In parallel, PBMC were activated by TransAct™ and cultured with IL2 till day 12. Three donors for each cell type were used. Cells were then stained for the panel of cytokine receptors and analyzed by flow cytometry.

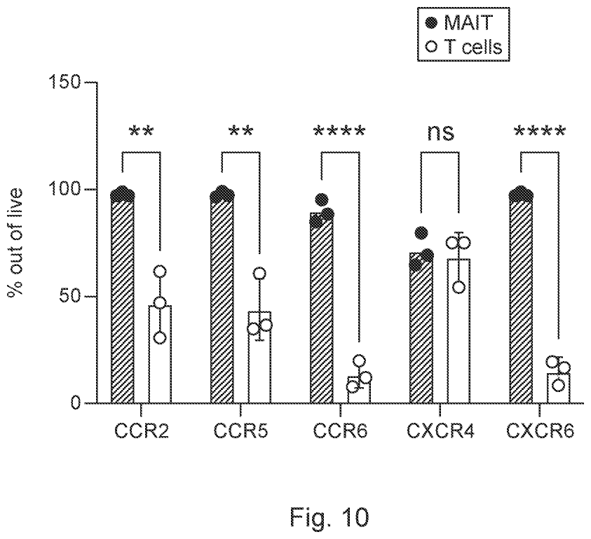

The results, presented in FIG. 10, demonstrate that the expression of chemokine receptors including CCR2, CCR5, CCR6 and CXCR6 is significantly higher on expanded MAIT cells as compared to expanded conventional T cells, whereas expression of CXCR4 is comparable.

### Example 6. MAIT Cells have Lower Allogeneic Profile than T Cells

A significant complication of using allogeneic cell compositions is their potential rejection by the immune system of the recipient host, and notably by peripheral blood T cells. Accordingly, MAIT cells and conventional T cells were compared with respect to their ability to induce allo-rejection by T cells from a non-related donor.

To this end, expanded MAIT cells and T cells (as described in Example 5 above) were pre-treated with mitomycin C for proliferation arrest and co-cultured with T cells from a non-related donor (“responder cells”) in a one-way mixed lymphocyte reaction (MLR). Potential allo-rejection was evaluated by expression of the activation marker CD25 on the responder cells, or by their proliferation rate.

The results are presented in FIG. 11. As can be seen, although responder cells reacted to MAIT cell, this reaction was significantly weaker than to conventional T cells, suggesting lower allo-rejection sensitivity of MAIT cells.

Of note, as demonstrated in FIG. 9B, MDR1 remains significantly up-regulated in expanded MAIT cells as compared to expanded conventional CD8⁺ T cells. Accordingly, the use of MAIT cell composition comprising ex-vivo expanded MAIT cells may provide for provide for enhanced retention and/or prolonged therapeutic activity, and even when administered to patients that are under treatment regimen with chemotherapies or other cancer treatments.

### Example 7. IVB-Derived MAIT Cells as Compared to MAIT Cells Derived from Cord Blood

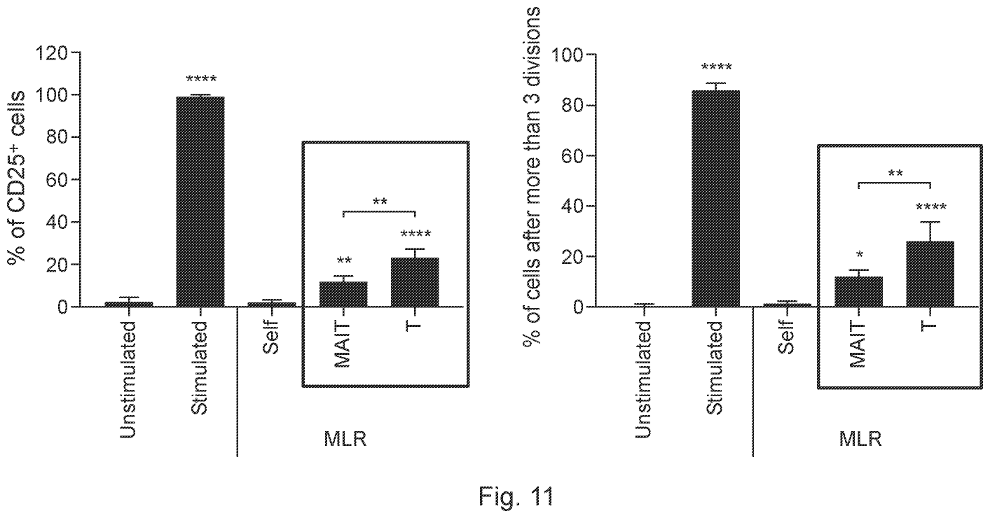

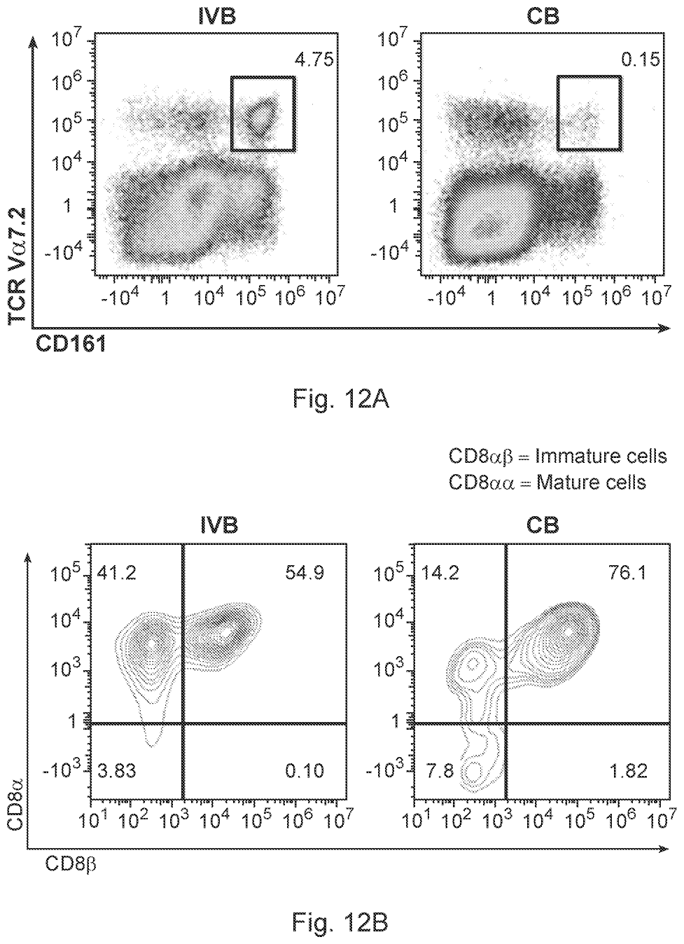

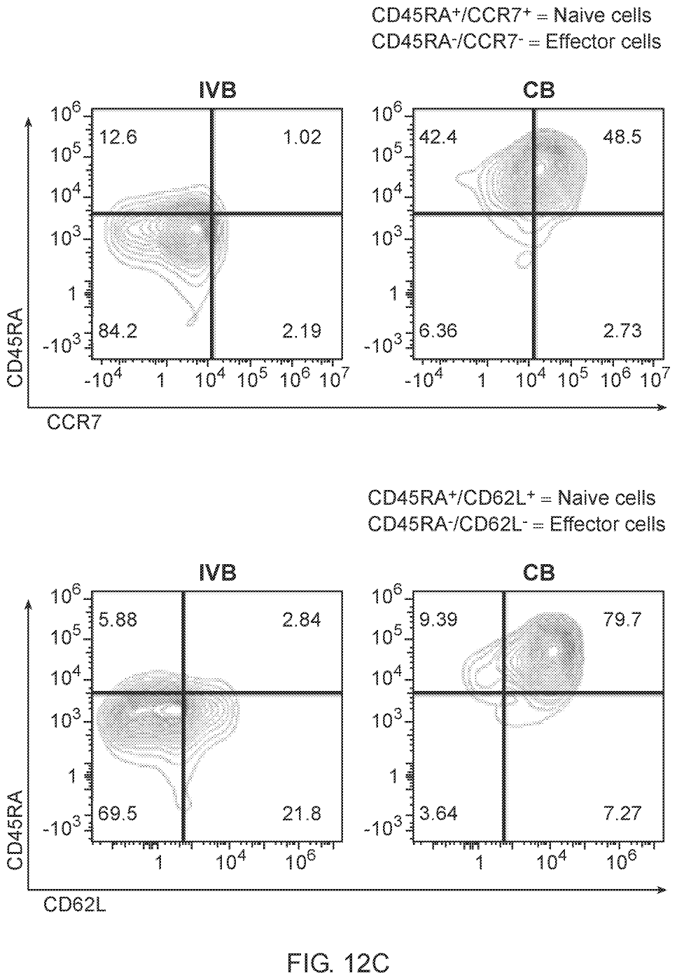

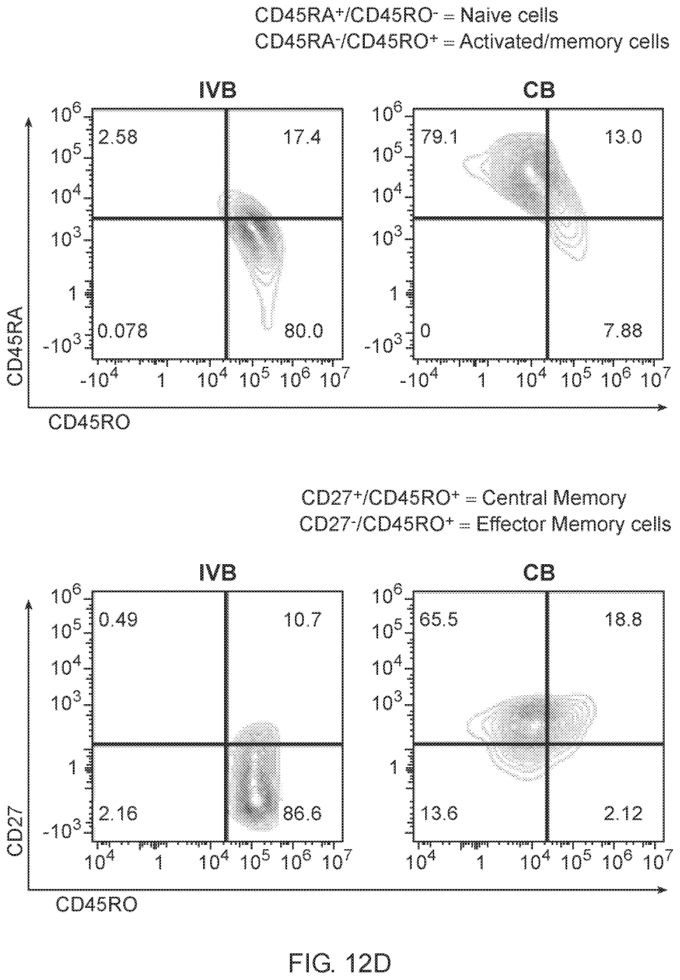

MAIT cells were collected from intervillous (IVB) and cord blood (CB) for frequency and immunophenotypic comparison between the two specimens by flow cytometry. As shown in FIG. 12A, there are very few MAIT cells in CB as compared to the MAIT cells in IVB (0.15% vs 4.75% of CD3⁺ gated cells). The data presented herein show high frequency of CD8αβ (immature) cell subpopulation in CB MAIT cells, accompanied by a relatively low percentage of CD8αα (mature) cells, indicating an immature/naïve state of CB MAIT cells as compared to the IVB-MAIT cells. (FIG. 12B). Accordingly, the CB MAIT cells display a naïve phenotype (CD45RA⁺CCR7⁺, CD45RA⁺CD62L⁺, CD45RA⁺CD45RO⁻) as compared to the MAIT cells in IVB which are mostly of effector memory phenotype (FIGS. 12C-12D).

In summary, comparing the percentages of MAIT cells in CB or IVB showed that the frequency of MAIT cells in CB is significantly lower than that in IVB. A lower frequency of CD8αα-expressing MAIT cells in CB indicates the CB MAIT cells are in an immature state relative to the mature MAIT cells in IVB. Increased frequency of effector memory MAIT cells was observed in IVB compared to their naïve state in CB as reflected by high percentage of cells that expressed CD45RA, CCR7, CD62L and CD27 markers in the CB MAIT cells. These data also indicate that the population of isolated IVB MAIT cells described herein does not have a significant contamination (if any) of MAIT cells from CB.

### Example 8. Generation of MAIT Cells Expressing an Exogenous TCR (eTCR)

For the generation of MAIT cells expressing an exogeneous TCR, a nucleic acid construct encoding an engineered TCR that recognizes NY-ESO-1 in the context of the HLA-A*0201 class I was used. The TCR construct encodes the human α and β chains having the amino acid sequence as set forth in SEQ ID NOs: 7 and 8, respectively. The construct contains the nucleic acid sequences encoding the α and β chains as set forth in SEQ ID NOs: 11 and 12, respectively, connected via a P2A linker comprising a 5′ furin recognition site. The encoded TCR is herein designated “NY-ESO-1 TCR” or “eTCR”. The resulting TCR recognizes a peptide epitope of SEQ ID NO: 9 in the context of HLA-A*0201 and/or HLA-A*0206.

The DNA sequence of the construct encoding the eTCR chains was cloned into the MSGV-1 retroviral expression vector, which is a derivative of the murine stem cell virus (MSCV)-based splice-gag vector (pMSGV that uses a MSCV long terminal repeat (LTR).

For the production of a composition comprise a population of placenta-derived NY-ESO-1-TCR⁺ MAIT cells, the following steps were performed:
1. Lymphocytes were isolated from IVB of human placentas as described above and cryopreserved for further use (isolation step).
2. Cells were thawed and cultured in lymphocyte medium supplemented with IL-15 (50 ng/ml) and 5-OP-RU (250 ng/ml) for at least 3 days (MAIT enrichment and activation step).
3. Activated cells were then transduced with the retroviral vector expressing the eTCR or with sham transduction (NT, cells incubated under the same conditions in the absence of a viral vectors) (transduction step).
4. Cells were grown in culture for a total of 10 days in the presence of IL-15 (expansion step).
5. At day 10, MAIT cells were separated by positive selection of TCRVα7.2-expressing cells (purification step).

The expansion rate and stability of vector expression were assessed at the end of the expansion step (Step 4). Following separation (Step 5), various experiments were conducted to evaluate the functional properties of the resulting composition.

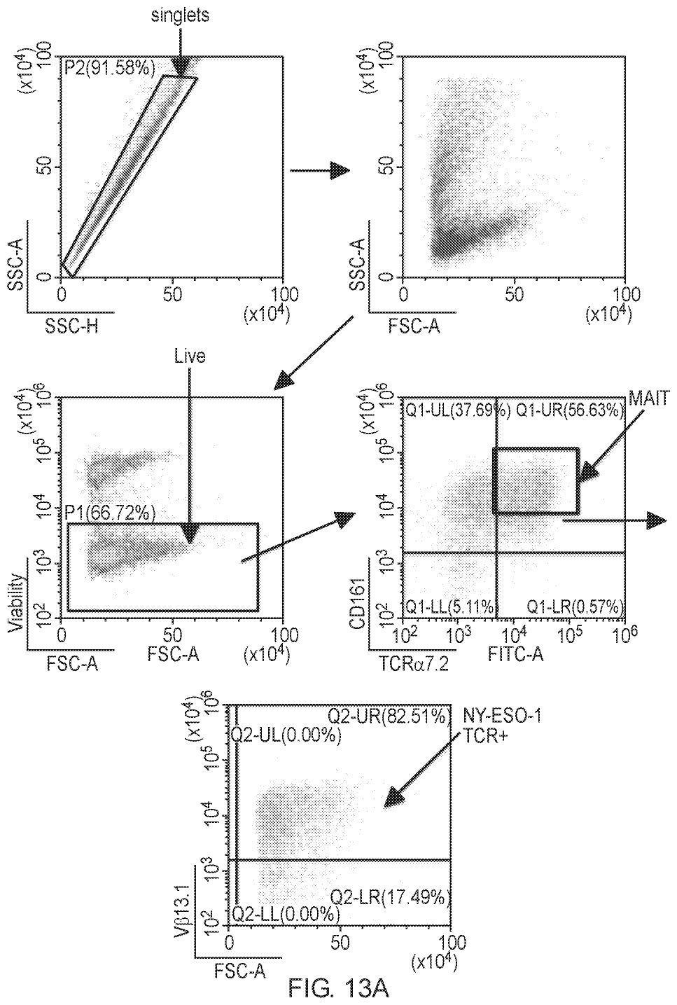

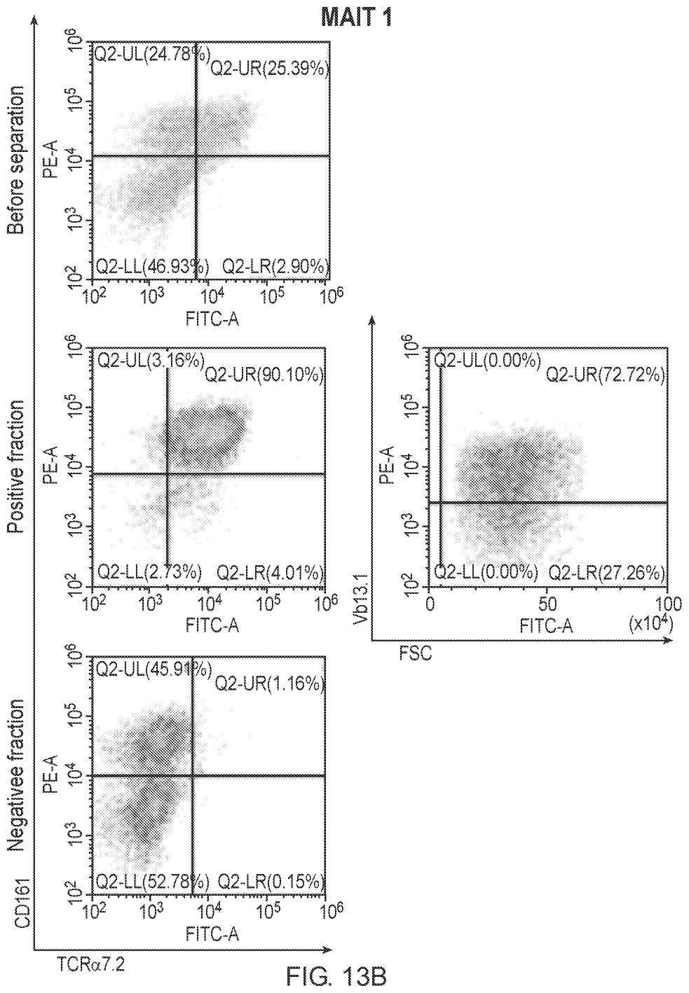

MAIT cells separated as described above, and stained for the markers CD161 and TCRVα7.2 to identify the percentage of cells exhibiting MAIT phenotype. Depicted in FIG. 13A is the flow cytometry gating strategy employed to evaluate MAIT cells phenotype as well as eTCR expression as tested by the Vβ13.1 antibody. Representative results are presented in FIG. 13B.

The results show that three batches tested for MAIT cell separation show an increased from 25%, 5% and 27% for CD161⁺TCRα7.2⁺ expression to 90%, 83% and 94% following the process described above. These purified MAIT cells expressed high levels of the exogenous NY-ESO-1 TCR (73%, 76% and 81% respectively, FIG. 13B). These results demonstrate that the process described can produce in high efficiency and specificity a composition comprising a population of eTCR⁺ MAIT cells.

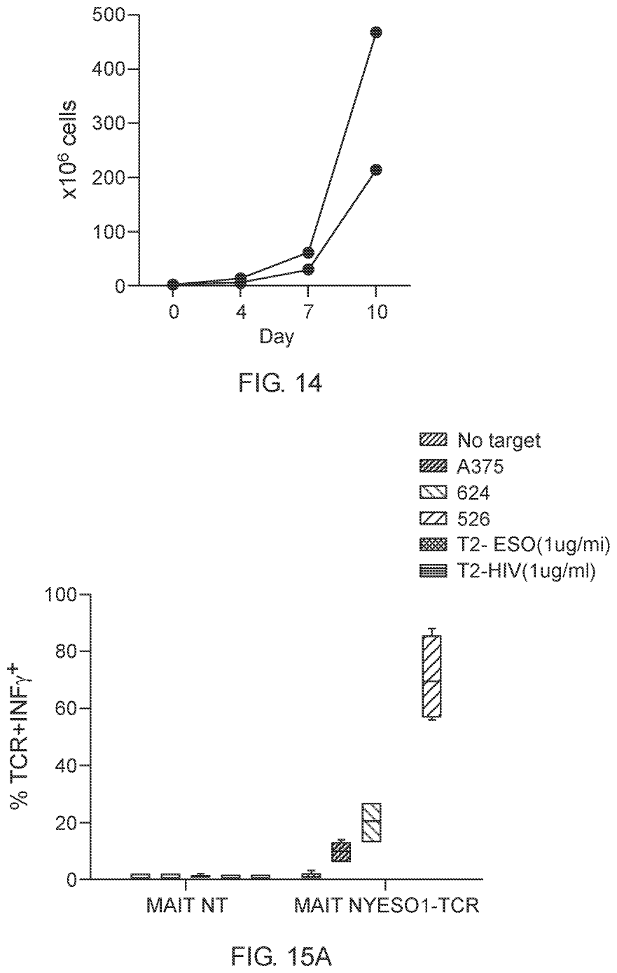

In addition, as can be seen in FIG. 14, MAIT cell as counted on days 0, 4, 7 and 10 showed a 275- and 102-fold increase in cell number in two separate batches, indicating high expansion and transduction abilities amenable for clinical use.

In comparison, alternative production protocols, in which the transduction step preceded the MAIT enrichment and activation step, provided lower cell survival and overall expansion rates.

### Example 9. HLA-I-Restricted Cytokine Response in eTCR⁺ MAIT Cells

To functionally test the resulting cell populations, compositions produced as described above were subjected to the following evaluations. Following separation at day 10, cytokine production and secretion were assessed (Example 9), as well as their activation and cytotoxicity levels (Example 10).

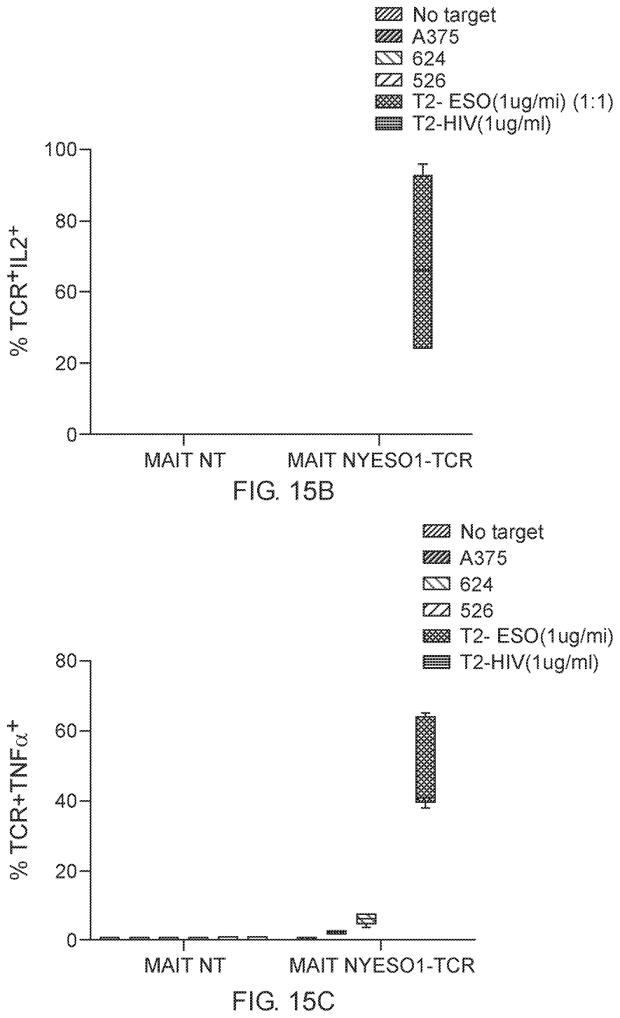

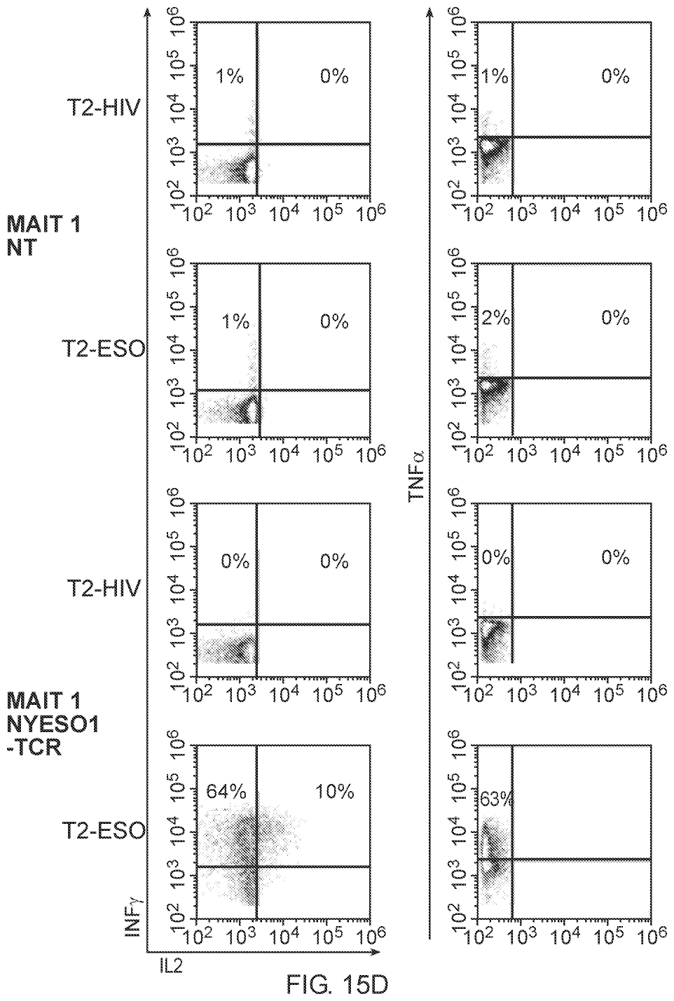

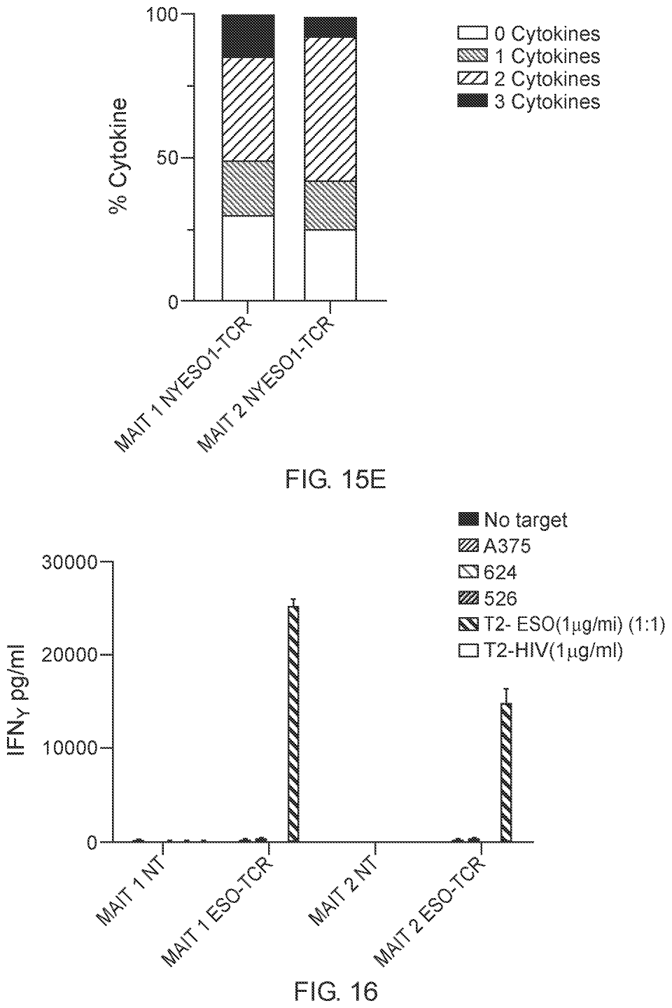

In order to test cytokine production and secretion, cells produced as described above were co-cultured with various target cell either expressing different levels of NY-ESO-1 (A375, M624, T2 cells loaded with 1 μg/ml of a NY-ESO-1 peptide) or negative for NY-ESO-1 (M526, T2 loaded with HIV peptide). For cytokine production capacities, cells cultured with target cells for 6 hours were analyzed for intracellular expression of IFNγ, IL-2 and TNFα. The experiment was done with an intracellular staining protocol using eBioscience™ Intracellular Fixation & Permeabilization Buffer Set and the corresponding anti-cytokine antibodies from Biolegend. The results are depicted in FIGS. 15A, 15B and 15C, respectively.

As can be seen in FIGS. 15A-15C, in two different MAIT batches, cells expressing the eTCR, but not non-transduced (NT) controls, showed cytokine expression upon recognition of their target antigen NY-ESO-1. As can further be seen, cytokine expression increased in a dose dependent manner on antigen expression on target, as evident form the differential response to cell types expressing different levels of the antigen Measured cytokine levels in MAIT cells expressing the eTCR increased to a maximum of 88% for IFNγ (FIG. 15A), 19% for IL-2 (FIG. 15B) and 65% for TNFα (FIG. 15C) upon co-culture with T2 cells loaded with NY-ESO-1 peptide (T2-ESO).

Representative plots of eTCR⁺ MAIT cells depicting the simultaneous staining of IL-2 with IFNγ or TNFα are presented in FIG. 15D. As can be seen, upon recognition of the target antigen NY-ESO-1, populations of cells co-expressing multiple cytokines can be identified. Simultaneous expression of multiple cytokines by eTCR⁺ MAIT cells demonstrating the poly-functional state of these cells is further illustrated in FIG. 15E. As can be seen, the results demonstrate more than 50% of the cells expressed at least two of these cytokines, and that at least 7% of them expressed all three tested cytokines

To further quantify IFNγ secretion from MAIT NY-ESO-1 TCR cells, ELISA of supernatants of MAIT cells co-cultured for 24 hours with target cells was performed according to the manufacturer's instructions (Human IFN-gamma DuoSet ELISA, R&D, DY285B-05). The results are presented in FIG. 16. As can be seen, in two separate batches, eTCR⁺ MAIT cells secreted high levels of IFNγ upon co-culture with A375, M624 and NY-ESO-1-loaded T2 cells. Secretion reached significant levels of 25,200 μg/ml and 14,900 μg/ml for the two MAIT batches when co-cultured with T2-ESO (FIG. 16).

Overall, these results demonstrate that MAIT cells transduced to express a NY-ESO-1-specific TCR are capable of antigen-induced cytokine expression and secretion. Furthermore, a poly-functional state was observed, indicating that these cells have a high functional potential.

### Example 10. Redirected Antigen Response-Activation Markers and Cytotoxicity

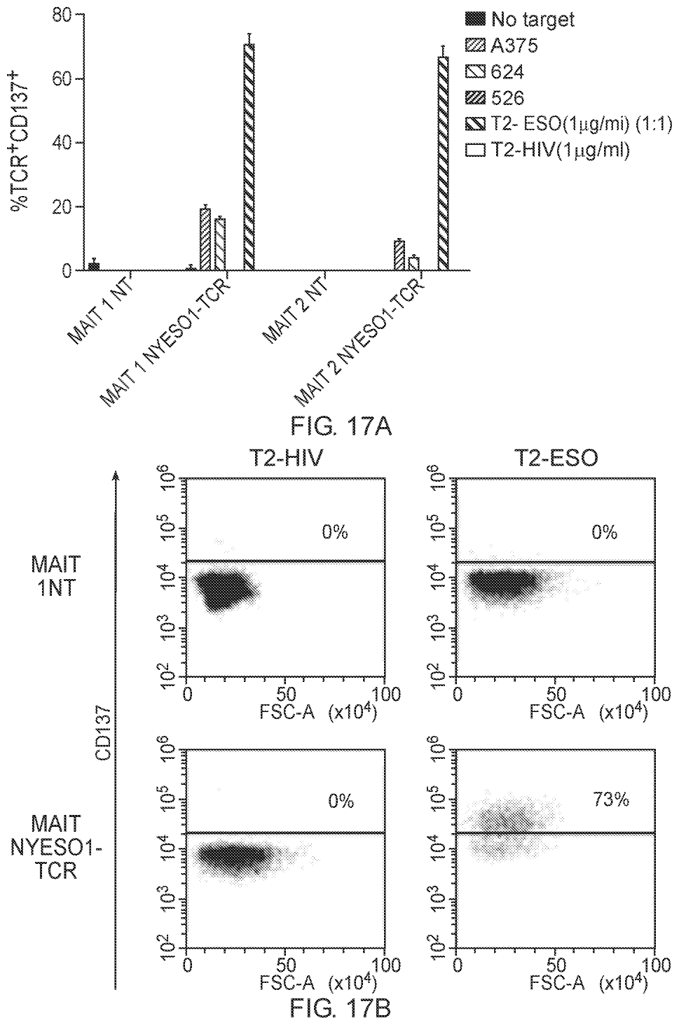

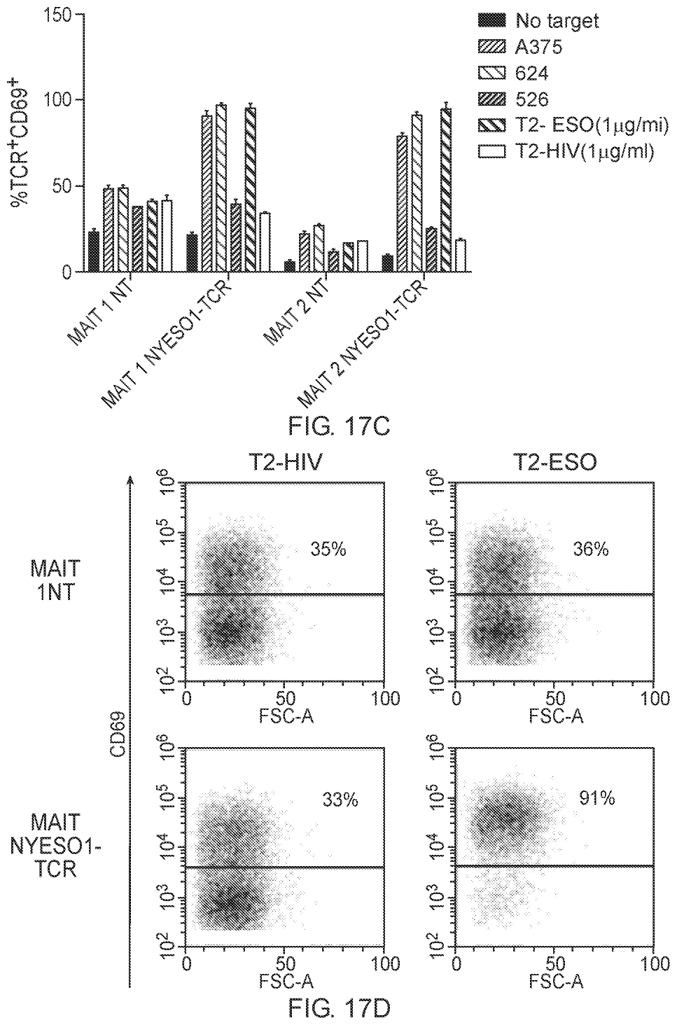

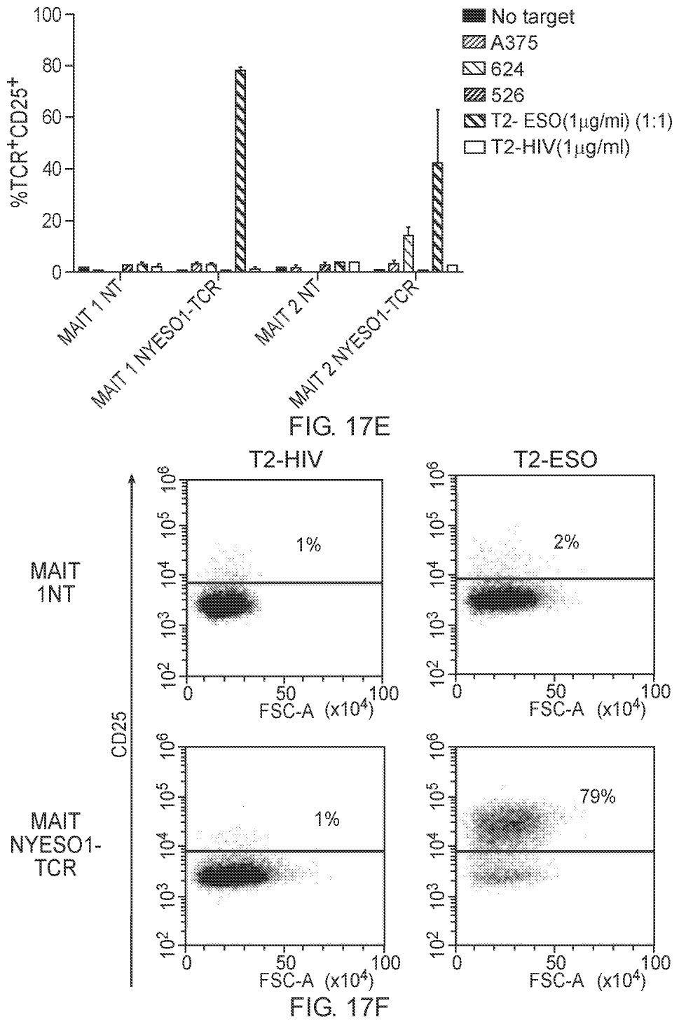

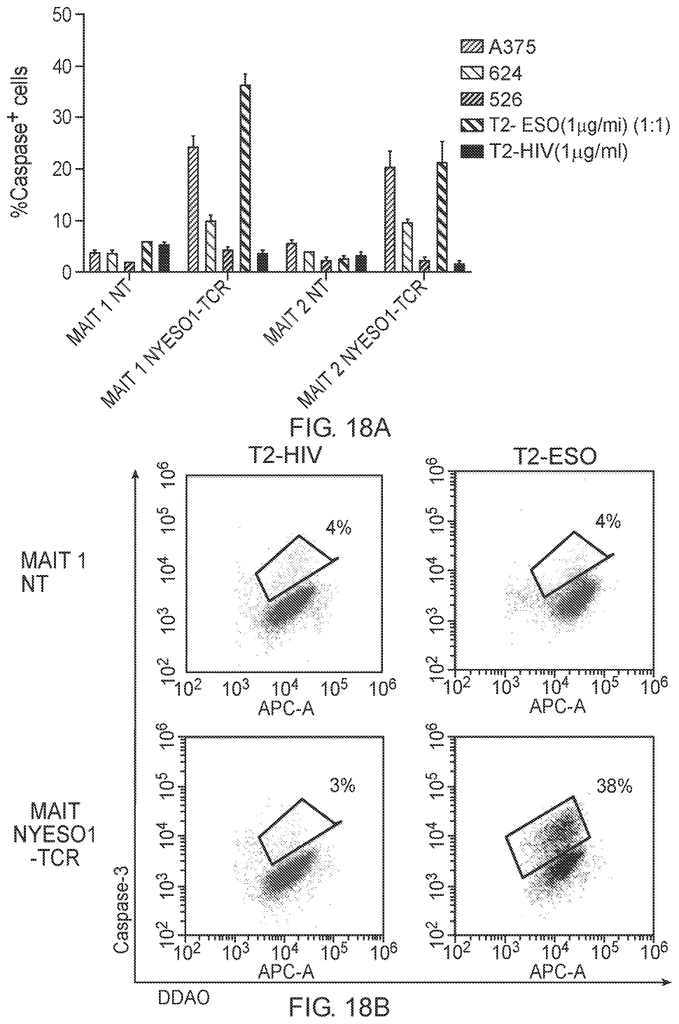

In order to test the activation of MAIT NY-ESO-1 TCR, cells were co-cultured with various target cells either expressing NY-ESO-1 (A375, M624, T2 cells loaded with NY-ESO-1 peptide) or negative for NY-ESO-1 (M526, T2 loaded with HIV peptide). Following 24 hours of incubation, surface expression of the markers CD137, CD69 and CD25 was measured by flow cytometry. The results are shown in FIGS. 17A-17F.

As can be seen, the activation markers CD137 and CD25 showed specific increase only when eTCR⁺ MAIT cells were co-cultured with NY-ESO-1-expressing target cells. The highest levels of these markers were obtained when co-cultured with T2-ESO cells, reaching 73% for CD137 and 79% for CD25 (FIGS. 17A-17B, 17E-17F). The marker CD69 also increased in eTCR⁺ MAIT cells upon target recognition from a baseline of 35% to 91% when co-cultured with T2-ESO cells (FIGS. 17C-17D). These results demonstrate that the NY-ESO-1-specific TCR introduced into MAIT cells can functionally activate these cells and cause them to express corresponding T cell activation markers.

Next, the cytotoxic potential of eTCR⁺ MAIT cells against various cell lines was measured. Killing was evaluated by the expression of active caspase-3 on target cells after co-culture with eTCR⁺ or control MAIT cells. Two batches of MAIT cells were tested, and the results are presented in FIGS. 18A-18B. As can be seen, MAIT cells expressing NY-ESO-1 TCR exhibited up to 38% caspase-3 expression in NY-ESO-1 positive target cells, with the highest killing observed in T2-ESO and A375 cells in both batches. Interestingly, the killing of M624 cells, expressing higher NY-ESO-1 levels, was also significant but to a lower extent than that of A375 cells, expressing lower levels of the antigen (FIGS. 18A-18B).

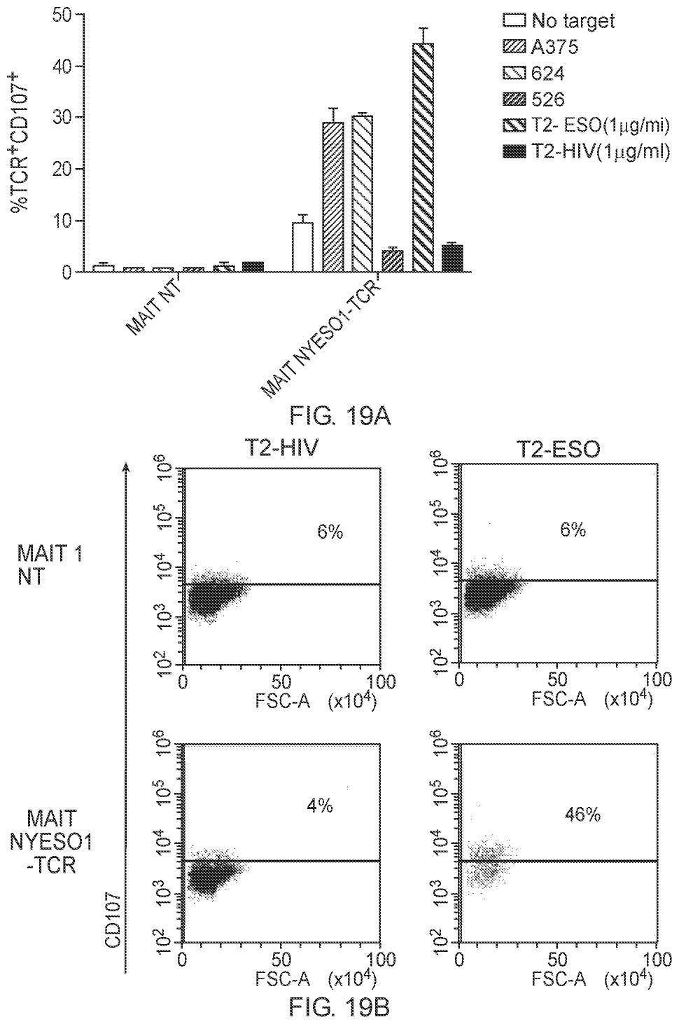

In addition to direct killing, cytotoxic capacity was also evaluated using surface expression of CD107, as indication of degranulation, as well as by granzyme B secretion (GZMB). The results are presented in FIGS. 19A-19B (CD107) and 20 (granzyme B).

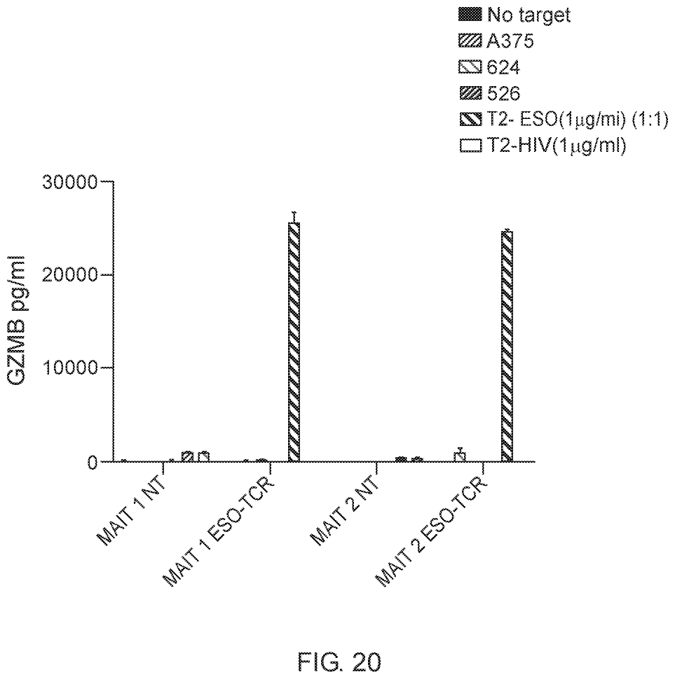

As can be seen, increased CD107 was detected in eTCR⁺ MAIT cells upon recognition of NY-ESO-1+ cells. An average of 43% of the eTCR⁺ MAIT population were positive for CD107 after co-culture with T2-ESO cells (FIGS. 19A-19B). In addition, granzyme B was secreted in high amounts in two batches of eTCR⁺ MAIT cells after co-culture with T2-ESO to a maximum level of 25,000 μg/ml (FIG. 20).

Overall, these results demonstrate that MAIT cells expressing NY-ESO-1 TCR have acquired the ability to target and perform direct specific lysis of cells expressing an HLA class I-restricted antigen, namely NY-ESO-1. Disclosed herein are particularly effective processes for the production of improved cell compositions for ACT, characterized by unique and advantageous properties.

It is noted, that cell compositions of the invention did not require high levels of antigen presentation in order to exert effective antigen-induced cytotoxicity. For example, M624 cells are known to present moderately low levels of NY-ESO-1 epitopes in the context of HLA-A2 (about 30-50 complexes per cell). In comparison, A375 cells are known to present lower levels of HLA-A2-presented NY-ESO-1 epitopes (about 10-30 complexes per cell), whereas 526 cells are characterized by levels of the HLA-A2-presented NY-ESO-1 epitopes that are below the detectability threshold (and insufficient for effective TCR-mediated activation). In the experiments described herein, MAIT cells transduced by a NY-ESO-1-specific TCR were specifically activated by low-antigen expressing A375 and M624 cells (˜10-50 antigen molecules per cell) and were able to kill these cells efficiently (as evident by cleaved caspase 3 expression in targets and indirectly by the expression of activation markers on TCR-transduced MAIT cells).

Notably, while the activation of the eTCR⁺ cells was enhanced in the presence of increasing antigen levels (as evident by activation markers and cytokine secretion in the presence of M624 cells as compared to A375 cells), specific lysis was unexpectedly enhanced in A375 cells as compared to M624 cells. In other words, direct cytotoxicity of eTCR⁺ MAIT cells was surprisingly found to particularly potent against tumor cells expressing (physiologically significant) low NY-ESO-1 levels than it was against tumor cells expressing higher levels of NY-ESO-1.

### Example 11. Isolation and Expansion of MAIT Cells from Intervillous Blood (IVB) in a Three-Dimensional (3D) System

To generate MAIT cells in a 3D system, a 0.5 L packed bed MiniBio reactor was assembled, containing 2.5 grams of Fibra-Cel® disks, then sterilized in autoclave by steam sterilization, at temperature of 122.5° C. and pressure of 1 bar over atmosphere pressure for 30 minutes. Afterwards the MiniBio reactor was connected to Applikon MiniBio control station. The Fibra-Cel® disks were incubated with RPMI-1640 supplemented with 10% HI-FBS for about 24 hours at 37° C., during the incubation the serum proteins interact electrostatically with the hydrophilic end group on the Fibra-Cel® disks and create an ECM coating on the Fibra-Cel®, which mimics cells natural environment.

300±20×10⁶ cells were thawed into 4Cell® Nutri-T GMP Medium supplemented with 1% L-Glutamine 200 mM and 0.1% 50 mg/ml gentamicin, the thawed cells were diluted to a target concentration of 1×10⁶ cells/ml. The prepared cells suspension was seeded into the bioreactor system set to the following conditions: temperature of 37° C., 80% DO, pH 7.4 and agitation of 100 rpm, to a final volume of 300 ml. Upon seeding, the cells were distributed within the bioreactor between the packed bed, and the “external” environment, and a decrease in cells concentration was observed 3 hours after seeding.

Three hours after seeding the MAIT cells were activated via their T cell receptor (TCR). Activation pathway through TCR requires co-stimulatory signal that includes a recognition of microbial-derived riboflavin metabolites presented on the MHC Class I-like molecule MR1, and co-stimulation by CD28, TLR agonists, bacterial products, or cytokines. The initial cells population, isolated from human placental IVB, includes various antigen-presenting cells (APC) such as dendritic cells, monocytes, B cells, which can activate MAIT cells via MR1. 5-OP-RU, which is a microbially-derived riboflavin intermediate, was added to the growth media at concentration of 250 nM to be presented by APC on MR1 to a MAIT cell TCR.

During the growth period, the growth media and cells suspension were sampled daily to measure pH, cells concentration (by Vi-Cell), cells metabolic activity according to nutrients consumption (by Cedex™ bio analyzer), and cells population distribution by flow cytometer (CytoFLEX™). The cells were grown in the packed bed bioreactor for 10 days, medium refreshments were conducted on days 3, 5 and 7 by 5, 5.2 and 100% respectively. On the 10th day of culture, the cells were harvested from the packed bed bioreactor as the maximal growth capacity has been achieved. The total cells number reached 1089×10⁶, 94% MAIT cells (Vα7.2 positive, CD161 high), meaning a fold expansion of 43.5. The proportions of the different cell populations within the culture and their variation over time are presented in Table 3.

After harvesting the cells from the bioreactor on the 10th day of the culture, further expansion of the cells was examined. An additional 0.5 L packed bed MiniBio reactor was assembled and prepared same as previous, a sterile system containing 2.5 grams of Fibra-Cel® disks, pre-incubated with RPMI-1640 supplemented with 10% HI-FBS for about 24 hours at 37° C., in order to provide ECM coating on the Fibra-Cel® discs and create a natural environment for the re-seeded cells. After ECM coating, the packed bed bioreactor was pre-equilibrated for the culture with growth media, composed of 4Cell® Nutri-T GMP Medium supplemented with 1% L-glutamine 200 mM and 0.1% 50 mg/ml gentamicin. 295×10⁶ of the harvested cells were seeded into the growth media, diluted to a target concentration of ˜1×10⁶ cells/ml. The prepared cells suspension was seeded into the bioreactor system set to the following conditions: temperature of 37° C., 80% DO, pH 7.4 and agitation of 100 rpm, to a final volume of 300 ml. IL-15 at concentration of 50 ng/ml was added to the growth media to induce MAIT cells expansion.

During the growth period, the growth media and cells suspension were sampled daily to measure pH, cells concentration (by Vi-Cell), cells metabolic activity according to nutrients consumption (by Cedex bio analyzer), and cells population distribution by flow cytometer (CytoFLEX™). The cells were grown in the packed bed bioreactor for 7 additional days, medium refreshments were conducted on days 12 and 14 of culture by 26.5 and 100% respectively. On the 17^{th} day of culture, the cells were harvested from the packed bed bioreactor. The total cells number reached 490×10⁶, 88% MAIT cells (Vα7.2 positive, CD161 high), meaning a fold expansion of 1.5. The relative percentage of MAIT cells within the culture and their variation over time is presented in Table 4.

Table 2 below summarizes the initial and final total viable cells, their relative population share and fold expansion during the growth period.

TABLE 2
characteristics of cells during growth period

| | Initial number | | Final number | | | Days |
|---|---|---|---|---|---|---|
| MAIT | of cells | | of cells | | Fold | of |
| Cells | [×10⁶ cells] | | [×10⁶ cells] | | expansion | growth |
| Days | 299.37 | (8% MAIT) | 1089 | (94% MAIT) | 43.5 | 10 |
| 1-10 | | | | | | |
| Days | 295 | (94% MAIT) | 490 | (86% MAIT) | 1.5 | 7 |
| 10-17 | | | | | | |

As shown in the above table, all fold expansion results are higher than 1, indicating cell expansion. In addition, the table shows a shift in cell population balance which reached over 94% of the target cell at the end of the examined growth period (MAIT cell after 10 growth days). The MAIT second growth period shows a slight decrease in MAIT cell percentage (from 94% to 86%).

TABLE 3
MAIT cell activation and proliferation
for 10 days in a packed bed bioreactor

| | Cells | CD3⁺ | | CD69⁺ | |
|---|---|---|---|---|---|
| Days of | out of | cells out | MAIT cells | cells out | CD25⁺ cells |
| culture | singlets | of live cells | out of CD3⁺ | of MAIT | out of MAIT |
| 0 | 18.34% | 37.30% | 22.63% | 8.57% | 0.64% |
| 5 | 29.20% | 74.43% | 46.01% | 59.51% | 98.06% |
| 7 | 30.44% | 93.45% | 89.86% | 77.68% | 99.57% |
| 10 | 76.34% | 96.93% | 96.26% | 32.05% | 46.33% |

The results presented in Table 3 demonstrate an increase in the proportion of MAIT cells starting with 22.63% on day 0 and up to 96.26% on day 10. In addition, the expression of activation markers CD69 and CD25 was elevated from day 0 to day 7, and decreased by day 10. “Cells out of singlets” represent the population of cells gated for further analyses.

TABLE 4
MAIT cell activation and proliferation following transition
from the first bioreactor to the second bioreactor cells

| | Cells | CD3⁺ | | CD69⁺ | CD25⁺ |
|---|---|---|---|---|---|
| Days post | out of | cells out | MAIT cells | cells out | cells out |
| harvest | singlets | of live cells | out of CD3⁺ | of MAIT | of MAIT |
| 10 | 80.57% | 97.15% | 95.22% | 30.60% | 36.44% |
| 12 | 81.42% | 95.20% | 96.46% | 45.21% | 15.94% |
| 14 | 64.09% | 96.56% | 95.04% | 70.55% | 10.01% |
| 17 | 29.86% | 92.52% | 81.85% | 87.30% | 7.86% |

The results presented in Table 4 demonstrate that MAIT percentages were similar during most of the time but presented a decrease from >90% at day 14 to 82% at day 17. CD69 marker expression was upregulated from 30% to 87% on day 17, indicating that MAIT cells maintained their activation signal, while CD25 gradually reduced.

In addition, the expression of chemokine receptors was examined over the course of MAIT cells activation and expansion as described in Example 5, as well as in MAIT grown in the bioreactor 3D system as described above. It was found that MAIT cells retain high expression of tissue-homing chemokine receptors including CCR5, CCR6 and CXCR6 at both day 10 and day 17 post-activation and same levels were kept on MAIT cultured in the 3D system.

### Example 12—Comparative Example of Engineered T Cells—IVB MAIT and PBMC-Derived

IBV-derived MAIT cells and donor-matched PBMC-derived control T cells (collected from the same donor) are obtained from placentas and blood of three healthy female donors. Cells in each sample are transduced with the NY-ESO-1-specific eTCR construct and expanded for 10 days as described above. Structural and functional parameters of the transduced cells are evaluated and comparted at different time points as detailed below.

On day 0 (prior to transduction and expansion), cells are incubated with labeled antibodies directed to various differentiation markers CD45RA, CCR7, CD62L (differentiation panel) and exhaustion markers PD1, TIM3, LAG3, CTLA4 (exhaustion panel) and evaluated by flow cytometry as described above. Activation and cytotoxicity markers 41BB, CD25, CD69, CD107 and GZMB are also evaluated by flow cytometry (activation and cytotoxicity FACS panel). In addition, transcription factors RORγt, t-bet and EOMES are evaluated by intra-nuclear staining with antibodies and flow cytometry (transcription panel). Transduction efficiency and purity are also evaluated by flow cytometry following transduction and separation using eTCR, MAIT and T cell-specific antibodies.

On day 10 (following expansion), the resulting cell compositions are evaluated again using the differentiation panel, exhaustion panel, activation and cytotoxicity FACS panel and transcription panel.

In addition, functional assays are performed by co-culturing the resulting cells with target cells (T2 loaded with NY-ESO-1 peptide, A375, 526, and M624 cells) essentially as described above. The following functional assays are performed:
Intracellular staining for cytokines IL-2, IFNγ and TNFα.
Specific killing—Caspase 3 staining in target cells.
Activation and cytotoxicity FACS panel—41BB, CD25, CD69, CD107 and GZMB.
ELISA for cytokine secretion-IFNγ, GZMB.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A cell composition comprising a population of engineered mucosal-associated invariant T (MAIT) cells expressing an exogenous T cell receptor (TCR), wherein the MAIT cells are derived from placenta, optionally further comprising a pharmaceutically acceptable carrier.

2. The cell composition of claim 1, wherein the MAIT cells are derived from placental intervillous blood (IVB).

3. The cell composition of claim 1, which is adapted for adoptive transfer cell therapy (ACT).

4. The cell composition of claim 1, which comprises 10⁹-10¹¹ viable cells of said engineered MAIT cell population.

5. The cell composition of claim 1, comprising at least 90% TCR Vα7.2⁺ CD161^{high} cells.

6. The cell composition of claim 1, wherein said TCR recognizes a tumor antigen.

7. The cell composition of claim 6, wherein the tumor antigen is selected from the group consisting of: NY-ESO-1, KRAS, p53, PIK3CA, PTEN, ERBB2 (HER2), AFP, KK-LC-1, RAC1-P29S, LAGE-1A, COL6A3, HA-2, HERV-E, BRAF, gp100, alpha-fetoprotein, Desmoyokin/AHNAKS2580F, Cancer/testis antigen 1, ERBB2H473Y, ERBB2IPE805G, minor H antigen (HA-1), PRAME, PSMA, TPBG, 5T4, MAGEA1, MAGE-A3/A6, MAGEA4/8, Melan-A/MART-1, NRAS and Wilms tumor 1 (WT-1).

8. The cell composition of claim 7, wherein said tumor antigen is selected from the group consisting of NY-ESO-1 and LAGE-1A.

9. The cell composition of claim 1, wherein said TCR is capable of specific binding to an HLA-A2-presented epitope.

10. The cell composition of claim 9, wherein said TCR comprises a TCR α chain comprising a CDR1 having the amino acid sequence of SEQ ID NO: 1, a CDR2 having the amino acid sequence of SEQ ID NO: 2, and a CDR3 having the amino acid sequence of SEQ ID NO: 3, and a TCR β chain comprising a CDR1 having the amino acid sequence of SEQ ID NO: 4, a CDR2 having the amino acid sequence of SEQ ID NO: 5, and a CDR3 having the amino acid sequence of SEQ ID NO: 6.

11. The cell composition of claim 10, wherein said TCR comprises a TCR α chain having the amino acid sequence as set forth in SEQ ID NO: 7, optionally excluding the signal peptide at positions 1-20 thereof, and a TCR β chain having the amino acid sequence as set forth in SEQ ID NO: 8, optionally excluding the signal peptide at positions 1-21 thereof.

12. (canceled)

13. (canceled)

14. (canceled)

15. (canceled)

16. (canceled)

17. (canceled)

18. A cell composition adapted for adoptive transfer cell therapy (ACT), the composition comprising a substantially purified population of mucosal-associated invariant T (MAIT) cells engineered to express an exogenous T cell receptor (TCR), wherein the TCR recognizes a tumor antigen selected from the group consisting of NY-ESO-1 and LAGE-1A.

19. The cell composition of claim 18, wherein said TCR comprises a TCR α chain comprising a CDR1 having the amino acid sequence of SEQ ID NO: 1, a CDR2 having the amino acid sequence of SEQ ID NO: 2, and a CDR3 having the amino acid sequence of SEQ ID NO: 3, and a TCR β chain comprising a CDR1 having the amino acid sequence of SEQ ID NO: 4, a CDR2 having the amino acid sequence of SEQ ID NO: 5, and a CDR3 having the amino acid sequence of SEQ ID NO: 6.

20. The cell composition of claim 19, wherein said TCR comprises a TCR α chain having the amino acid sequence as set forth in SEQ ID NO: 7, optionally excluding the signal peptide at positions 1-20 thereof, and a TCR β chain having the amino acid sequence as set forth in SEQ ID NO: 8, optionally excluding the signal peptide at positions 1-21 thereof.

21. (canceled)

22. (canceled)

23. A method of treating a subject afflicted with a tumor or malignancy associated with expression of an HLA class I restricted-antigen, comprising administering to the subject a cell composition comprising a population of engineered placental mucosal-associated invariant T (MAIT) cells expressing an exogenous T cell receptor (TCR), and a pharmaceutically acceptable carrier.

24. The method of claim 23, wherein the MAIT cells have been obtained from placental intervillous blood (IVB).

25. The method of claim 23, wherein said TCR recognizes a tumor antigen expressed by cells of the tumor or malignancy.

26. The method of claim 25, wherein the antigen is selected from the group consisting of NY-ESO-1 and LAGE-1A.

27. (canceled)

28. The method of claim 26, wherein said subject is HLA-A2-positive and is afflicted with a tumor or malignancy expressing NY-ESO-1 and/or LAGE-1A, and said TCR comprises a TCR α chain comprising a CDR1 having the amino acid sequence of SEQ ID NO: 1, a CDR2 having the amino acid sequence of SEQ ID NO: 2, and a CDR3 having the amino acid sequence of SEQ ID NO: 3, and a TCR β chain comprising a CDR1 having the amino acid sequence of SEQ ID NO: 4, a CDR2 having the amino acid sequence of SEQ ID NO: 5, and a CDR3 having the amino acid sequence of SEQ ID NO: 6.

29. The method of claim 28, wherein said TCR comprises a TCR α chain having the amino acid sequence as set forth in SEQ ID NO: 7, optionally excluding the signal peptide at positions 1-20 thereof, and a TCR β chain having the amino acid sequence as set forth in SEQ ID NO: 8, optionally excluding the signal peptide at positions 1-21 thereof.

30. The method of claim 28, wherein the tumor is selected from the group consisting of: melanoma, myeloma, sarcoma, and bladder, brain, ovarian, lung, breast, synovial and prostate tumors.

31. The method of claim 30, wherein said tumor is melanoma.

32. The method of claim 23, wherein said antigen is a low-density antigen characterized by surface expression of less than 50 antigen molecules per cell.

33. The method of claim 23, wherein the cell composition is adapted for adoptive transfer cell therapy (ACT) and the population comprises at least 10⁹ viable cells, of which at least 90% are TCR-Vα7.2⁺ CD161^{high}.

34. The method of claim 23, wherein the engineered MAIT cells are allogeneic to said subject.

35. The method of claim 34, wherein the engineered MAIT cells are partly histocompatible with said subject, or wherein the engineered MAIT cells are not histocompatible with said subject.

36. The method of claim 23, wherein the subject is afflicted with a treatment-resistant tumor or is not otherwise amenable for treatment with an immunotherapy comprising chimeric antigen receptor (CAR) T cells and/or therapeutic antibodies.

37. (canceled)

38. (canceled)

39. (canceled)

40. (canceled)

41. (canceled)

42. (canceled)

43. A method of treating or reducing the incidence of a cancer using an immunotherapeutic composition, the method comprising:
administering to said subject the cell composition of claim 6.

44. A method of treating or reducing the incidence of a cancer using an immunotherapeutic composition, the method comprising:
administering to said subject the cell composition of claim 18.

45. A method of treating or reducing the incidence of a cancer using an immunotherapeutic composition, the method comprising:
administering to said subject the cell composition of claim 23.


SEQUENCE LISTING (18 sequences)

SEQ ID NO: 1 - protein, 6 aa, synthetic construct
DSAIYN

SEQ ID NO: 2 - protein, 7 aa, synthetic construct
IQSSQRE

SEQ ID NO: 3 - protein, 13 aa, synthetic construct
AVRPLYGGSYIPT

SEQ ID NO: 4 - protein, 5 aa, synthetic construct
MNHEY

SEQ ID NO: 5 - protein, 6 aa, synthetic construct
SVGAGI

SEQ ID NO: 6 - protein, 12 aa, synthetic construct
ASSYVGNTGELF

SEQ ID NO: 7 - protein, 275 aa, synthetic construct
MFETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 8 - protein, 309 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 9 - protein, 9 aa, synthetic construct
SLLMWITQC

SEQ ID NO: 10 - protein, 5 aa, synthetic construct
LVIVL

SEQ ID NO: 11 - DNA, 825 bp, synthetic construct
  source 1..825: mol_type = other DNA
atgttcgaaactctgctggggctgctgattctgtggctgcagctgcagtgggtgtcatccaaacaggaggtcactcagattcccgctgccctgagcgtgcctgagggcgagaacctggtgctgaattgctccttcaccgactctgccatctacaacctgcagtggtttaggcaggatccaggcaagggcctgaccagcctgctgctgatccagagctcgcagagggagcagacatccggccgcctgaatgcctctctggacaagtctagcggccggagcaccctgtacatcgcagcaagccagccaggcgattccgccacatacctgtgcgccgtgcggcctctgtacggaggctcttatatcccaaccttcggcagaggcacaagcctgatcgtgcacccttacatccagaacccagaccccgccgtgtatcagctgcgggacagcaagtcctctgataagtccgtgtgcctgttcaccgactttgattcccagacaaacgtgagccagagcaaggactctgacgtgtacatcaccgacaagtgcgtgctggatatgagaagcatggactttaagtccaactctgccgtggcctggagcaataagtccgatttcgcctgcgccaacgcgtttaacaatagcatcatccccgaggatacattctttccttccccagagagctcctgtgacgtgaagctggtggagaagagcttcgagacagatacaaacctgaattttcagaacctgctggtcatcgtgctgcggatcctgctgctgaaggtggccggcttcaatctgctgatgaccctgagactgtggtctagc

SEQ ID NO: 12 - DNA, 930 bp, synthetic construct
  source 1..930: mol_type = other DNA
atgtctatcggcctgctgtgctgtgccgccctgagcctgctgtgggcaggaccagtgaacgcaggagtgacccagacacccaagttccaggtgctgaagaccggccagtctatgacactgcagtgcgcccaggacatgaatcacgagtacatgagctggtatcggcaggatcctggcatgggcctgagactgatccactactccgtgggagcaggaatcaccgaccagggagaggtgccaaacggctataacgtgagcaggagcaccacagaggatttcccactgaggctgctgtctgccgcaccttctcagacaagcgtgtacttttgcgcctcctcttatgtgggcaacaccggcgagctgttctttggcgagggctccaggctgacagtgctggaggacctgaataaggtgttcccccctgaggtggccgtgtttgagccctctgaggccgagatcagccacacccagaaggccaccctggtgtgcctggcaaccggcttctttcctgatcacgtggagctgtcctggtgggtgaacggcaaggaggtgcactctggcgtgtgcacagacccacagcccctgaaggagcagccagccctgaatgactcgagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 13 - DNA, 18 bp, synthetic construct
  source 1..18: mol_type = other DNA
gactctgccatctacaac

SEQ ID NO: 14 - DNA, 21 bp, synthetic construct
  source 1..21: mol_type = other DNA
atccagagctcgcagagggag

SEQ ID NO: 15 - DNA, 39 bp, synthetic construct
  source 1..39: mol_type = other DNA
gccgtgcggcctctgtacggaggctcttatatcccaacc

SEQ ID NO: 16 - DNA, 15 bp, synthetic construct
  source 1..15: mol_type = other DNA
atgaatcacgagtac

SEQ ID NO: 17 - DNA, 18 bp, synthetic construct
  source 1..18: mol_type = other DNA
tccgtgggagcaggaatc

SEQ ID NO: 18 - DNA, 36 bp, synthetic construct
  source 1..36: mol_type = other DNA
gcctcctcttatgtgggcaacaccggcgagctgttc